(19) United States
(12) Patent Application Publication
(10) Pub. No.: US 2011/0230366 A1
(43) Pub. Date: Sep. 22, 2011

(54) Genetic Variants Useful for Risk Assessment of Thyroid Cancer

(75) Inventors: Julius Gudmundsson, Reykjavik (IS); Daniel Gudbjartsson, Reykjavik (IS); Patrick Sulem, Reykjavik (IS)

(73) Assignee: deCODE Genetics ehf., Reykjavik (IS)

(21) Appl. No.: 13/058,790

(22) PCT Filed: Aug. 12, 2009
(86) PCT No.: PCT/IS2009/000010

§ 371 Date: Feb. 11, 2011

(30) Foreign Application Priority Data
Aug. 12, 2008 (IS) 8755
Feb. 5, 2009 (IS) 8791

(51) Int. Cl. C40B 30/04; C12Q 1/68; G01N 33/53; G06F 19/00

(52) U.S. Cl. 506/9; 435/6.11; 436/501; 702/19

(57) ABSTRACT

The invention discloses genetic variants that have been determined to be susceptibility variants of thyroid cancer. Methods of disease management, including determining increased susceptibility to thyroid cancer, methods of predicting response to therapy and methods of predicting prognosis of thyroid cancer using such variants are described. The invention further relates to kits useful in the methods of the invention.

### INTRODUCTION

### Thyroid Cancer

Thyroid carcinoma is the most common classical endocrine malignancy, and its incidence has been rising rapidly in the US as well as other industrialized countries over the past few decades. Thyroid cancers are classified histologically into four groups: papillary, follicular, medullary, and undifferentiated or anaplastic thyroid carcinomas (DeLellis, R. A., J Surg Oncol, 94, 662 (2006)). Papillary and follicular carcinomas (including the Hürthle-cell variant) are collectively known as differentiated thyroid cancers, and they account for approximately 95% of incident cases (DeLellis, R. A., J Surg Oncol, 94, 662 (2006)). In 2008, it is expected that over 37,000 new cases will be diagnosed in the US, about 75% of them being females (the ratio of males to females is 1:3.2) (Jemal, A., et al., Cancer statistics, 2008. CA Cancer J Clin, 58: 71-96, (2008)). If diagnosed at an early stage, thyroid cancer is a well manageable disease with a 5-year survival rate of 97% among all patients, yet it is expected that close to 1,600 individuals will die from this disease in 2008 in the US (Jemal, A., et al., Cancer statistics, 2008. CA Cancer J Clin, 58: 71-96, (2008)). Survival rate is poorer (˜40%) among individuals that are diagnosed with a more advanced disease; i.e. individuals with large, invasive tumors and/or distant metastases have a 5-year survival rate of ≈40% (Sherman, S. I., et al., 3rd, Cancer, 83, 1012 (1998), Kondo, T., Ezzat, S., and Asa, S. L., Nat Rev Cancer, 6, 292 (2006)). For radioiodine-resistant metastatic disease there is no effective treatment and the 10-year survival rate among these patients is less than 15% (Durante, C., et al., J Clin Endocrinol Metab, 91, 2892 (2006)). Thus, there is a need for better understanding of the molecular causes of thyroid cancer progression to develop new diagnostic tools and better treatment options.

Although relatively rare (1% of all malignancies in the US), the incidence of thyroid cancer more than doubled between 1984 and 2004 in the US; due almost entirely to an increase in papillary thyroid carcinoma diagnoses (SEER web report; Ries L, Melbert D, Krapcho M et al (2007) SEER cancer statistics review, 1975-2004. National Cancer Institute, Bethesda, Md., http://seer.cancer.gov/csr/1975_{—}2004/, based on November 2006 SEER data submission). Between 1995 and 2004, thyroid cancer was the third fastest growing cancer diagnosis, behind only peritoneum, omentum, and mesentery cancers and “other” digestive cancers [SEER web report]. Similarly dramatic increases in thyroid cancer incidence have also been observed in Canada, Australia, Israel, and several European countries (Liu, S., et al., Br J Cancer, 85, 1335 (2001), Burgess, J. R., Thyroid, 12, 141 (2002), Lubina, A., et al., Thyroid, 16, 1033 (2006), Colonna, M., et al., Eur J Cancer, 38, 1762 (2002), Leenhardt, L., et al., Thyroid, 14, 1056 (2004), Reynolds, R. M., et al., Clin Endocrinol (Oxf), 62, 156 (2005), Smailyte, G., et al., BMC Cancer, 6, 284 (2006)). The factors underlying this epidemic are not well understood. In the apparent absence of increases in known risk factors, scientists have widely speculated that changing diagnostic practices may be responsible (Davies, L. and Welch, H. G., Jama, 295, 2164 (2006), Verkooijen, H. M., et al., Cancer Causes Control, 14, 13 (2003)).

The primary known risk factor for thyroid cancer is radiation exposure. Potential sources of exposure include radiation used in diagnostic and therapeutic medicine, as well as radioactive fallout from nuclear explosions. However, neither source appears to have increased over the past two decades in the US. Radiation therapy to the head and neck for benign childhood conditions, once common in the US, declined after the early 1950s (Zheng, T., et al., Int J Cancer, 67, 504 (1996)). Similarly, atmospheric testing of nuclear weapons in the United States ceased in 1963 with the signing of the Limited Test Ban Treaty. The effect of such nuclear testing on thyroid cancer rates, though not entirely clear, is thought to be limited (Gilbert, E. S., et al., J Natl Cancer Inst, 90, 1654 (1998), Hundahl, S. A., CA Cancer J Clin, 48, 285 (1998), Robbins, J. and Schneider, A. B., Rev Endocr Metab Disord, 1, 197 (2000)).

The rise in thyroid cancer incidence might be attributable to increased detection of sub-clinical cancers, as opposed to an increase in the true occurrence of thyroid cancer (Davies, L. and Welch, H. G., Jama, 295, 2164 (2006)). Thyroid cancer incidence within the US has been rising for several decades, yet mortality has stayed relatively constant (Davies, L. and Welch, H. G., Jama, 295, 2164 (2006)). The introduction of ultrasonography and fine-needle aspiration biopsy in the 1980s improved the detection of small nodules and made cytological assessment of a nodule more routine (Rojeski, M. T. and Gharib, H., N Engl J Med, 313, 428 (1985), Ross, D. S., J Clin Endocrinol Metab, 91, 4253 (2006)). This increased diagnostic scrutiny may allow early detection of potentially lethal thyroid cancers. However, several studies report thyroid cancers as a common autopsy finding (up to 35%) in persons without a diagnosis of thyroid cancer (Bondeson, L. and Ljungberg, O., Cancer, 47, 319 (1981), Harach, H. R., et al., Cancer, 56, 531 (1985), Solares, C. A., et al., Am J Otolaryngol, 26, 87 (2005) and Sobrinho-Simoes, M. A., Sambade, M. C., and Goncalves, V., Cancer, 43, 1702 (1979)). This suggests that many people live with sub-clinical forms of thyroid cancer which are of little or no threat to their health.

The somatic genetic defects believed to be responsible for PTC initiation have been identified in the majority of cases; these include genetic rearrangements involving the tyrosine kinase domain of RET and activating mutations of BRAF and RAS (Kondo, T., Ezzat, S., and Asa, S. L., Nat Rev Cancer, 6, 292 (2006), Tallini, G., Endocr Pathol, 13, 271 (2002)., Fagin, J. A., Mol Endocrinol, 16, 903 (2002)). Although some correlation studies support an association between specific genetic alterations and aggressive cancer behavior (Nikiforova, M. N., et al., J Clin Endocrinol Metab, 88, 5399 (2003), Trovisco, V., et al., J Pathol, 202, 247 (2004), Garcia-Rostan, G., at al., J Clin Oncol, 21, 3226 (2003), Nikiforov, Y. E., Endocr Pathol, 13, 3 (2002)), there are a number of events that are found nearly exclusively in aggressive PTCs, including mutations of P53 (Fagin, J. A., et al., J Clin Invest, 91, 179 (1993), La Perle, K. M., et al., Am J Pathol, 157, 671 (2000)), dysregulated β-catenin signaling (Karim, R., et al., Pathology, 36, 120 (2004)), up-regulation of cyclin D1 (Khoo, M. et al., J Clin Endocrinol Metab, 87, 1810 (2002)), and overexpression of metastasis-promoting, angiogenic, and/or cell adhesion-related genes (Klein, M., et al., J Clin Endocrinol Metab, 86, 656 (2001), Yu, X. M., et al., Clin Cancer Res, 11, 8063 (2005), Guarino, V., et al., J Clin Endocrinol Metab, 90, 5270 (2005), Brabant, G., et al., Cancer Res, 53, 4987 (1993), Scheumman, G. F., et al., J Clin Endocrinol Metab, 80, 2168 (1995), Maeta, H., Ohgi, S., and Terada, T., Virchows Arch, 438, 121 (2001) and Shiomi, T. and Okada, Y., Cancer Metastasis Rev, 22, 145 (2003)). It has also been demonstrated that invasive regions of primary PTCs are frequently characterized by enhanced Akt activity and cytosolic p27 localization (Ringel, M. D., et al., Cancer Res, 61, 6105 (2001), Vasko, V., et al., J Med Genet, 41, 161 (2004)). The functional roles for PI3 kinase, Akt, and p27 in PTC cell invasion in vitro has also been demonstrated (Guarino, V., et al., J Clin Endocrinol Metab, 90, 5270 (2005), Vitagliano, D., et al., Cancer Res, 64, 3823 (2004), Motti, M. L., et al., Am J Pathol, 166, 737 (2005)). However, the correlation between increased Akt activity and invasion was not found for PTCs with activating BRAF mutations. Most importantly, these focused studies do not address the more global question of which biological functions and signaling pathways are altered in invasive PTC cells.

### Medullary Thyroid Cancer

Of all thyroid cancer cases, 2% to 3% are of the medullary type (medullary thyroid cancer MTC) (Hundahl, S. A., et al., Cancer, 83, 2638 (1998)). Average survival for MTC is lower than that for more common thyroid cancers, e.g., 83% 5-year survival for MTC compared to 90% to 94% 5-year survival for papillary and follicular thyroid cancer (Hundahl, S. A., et al., Cancer, 83, 2638 (1998), Bhattacharyya, N., Otolaryngol Head Neck Surg, 128, 115 (2003)). Survival is correlated with stage at diagnosis, and decreased survival in MTC can be accounted for in part by a high proportion of late-stage diagnoses (Hundahl, S. A., et al., Cancer, 83, 2638 (1998), Bhattacharyya, N., Otolaryngol Head Neck Surg, 128, 115 (2003), Modigliani, E., et al., J Intern Med, 238, 363 (1995)). A Surveillance, Epidemiology, and End Results (SEER) population-based study of 1,252 medullary thyroid cancer patients found that survival varied by extent of local disease. For example, the 10-year survival rates ranged from 95.6% for disease confined to the thyroid gland to 40% for those with distant metastases (Roman, S., Lin, R., and Sosa, J. A., Cancer, 107, 2134 (2006)).

MTC arises from the parafollicular calcitonin-secreting cells of the thyroid gland. MTC occurs in sporadic and familial forms and may be preceded by C-cell hyperplasia (CCH), though CCH is a relatively common abnormality in middle-aged adults. In a population-based study in Sweden, 26% of patients with MTC had the familial form (Bergholm, U., Bergstrom, R., and Ekbom, A., Cancer, 79, 132 (1997)). A French national registry and a U.S. clinical series both reported a higher proportion of familial cases (43% and 44%, respectively) (Modigliani, E., et al., J Intern Med, 238, 363 (1995), Kebebew, E., et al., Cancer, 88, 1139 (2000)). Familial cases often indicate the presence of multiple endocrine neoplasia type 2, a group of autosomal dominant genetic disorders caused by inherited mutations in the RET proto-oncogene (OMIM, online mendelian inheritance in men (http://www.ncbi.nlm.nih.gov/sites/entrez?db=omim)).

### Anaplastic Thyroid Cancer

Anaplastic tumors are the least common (about 0.5 to 1.5%) and most deadly of all thyroid cancers. This cancer has a very low cure rate with the very best treatments allowing only 10% of patients to be alive 3 years after it is diagnosed. Most patients with anaplastic thyroid cancer do not live one year from the day they are diagnosed. Anaplastic thyroid cancer often arises within a more differentiated thyroid cancer or even within a goiter. Like papillary cancer, anaplastic thyroid cancer may arise many years (>20) following radiation exposure. Cervical metastasis (spread of the cancer to lymph nodes in the neck) are present in the vast majority (over 90%) of cases at the time of diagnosis. The presence of lymph node metastasis in these cervical areas causes a higher recurrence rate and is predictive of a high mortality rate (Endocrine web, (http://www.endocrineweb.com/caana.html)).

Genetic risk is conferred by subtle differences in the genome among individuals in a population. Genomic differences between individuals are most frequently due to single nucleotide polymorphisms (SNP), although other variations, such as copy number variations (CNVs) are also important. SNPs are located on average every 1000 base pairs in the human genome. Accordingly, a typical human gene containing 250,000 base pairs may contain 250 different SNPs. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphism in the human genome is caused by insertions, deletions, translocations, or inversions of either short or long stretches of DNA. Genetic polymorphisms conferring disease risk may therefore directly alter the amino acid sequence of proteins, may increase the amount of protein produced from the gene, or may decrease the amount of protein produced by the gene.

As genetic polymorphisms conferring risk of common diseases are uncovered, genetic testing for such risk factors is becoming important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

There is an unmet need for genetic variants that confer susceptibility of thyroid cancer. Such variants are expected to be useful for risk management of thyroid cancer, based on the utility that individuals at particular risk of developing thyroid cancer can be identified. The present invention provides such susceptibility variants.

### SUMMARY OF THE INVENTION

The present invention relates to methods of risk management of thyroid cancer, based on the discovery that certain genetic variants are correlated with risk of thyroid cancer. Thus, the invention includes methods of determining an increased susceptibility or increased risk of thyroid cancer, as well as methods of determining a decreased susceptibility of thyroid cancer, through evaluation of certain markers that have been found to be correlated with susceptibility of thyroid cancer in humans. Other aspects of the invention relate to methods of assessing prognosis of individuals diagnosed with thyroid cancer, methods of assessing the probability of response to a therapeutic agents or therapy for thyroid cancer, as well as methods of monitoring progress of treatment of individuals diagnosed with thyoroid cancer.

In one aspect, the present invention relates to a method of diagnosing a susceptibility to thyroid cancer in a human individual, the method comprising determining the presence or absence of at least one allele of at least one polymorphic marker on selected from rs965513 (SEQ ID NO:1), and markers in linkage disequilibrium therewith, in a nucleic acid sample obtained from the individual, wherein the presence of the at least one allele is indicative of a susceptibility to thyroid cancer. The invention also relates to a method of determining a susceptibility to thyroid cancer, by determining the presence or absence of at least one allele of at least one polymorphic selected from rs965513 (SEQ ID NO:1), and markers in linkage disequilibrium therewith, wherein the determination of the presence of the at least one allele is indicative of a susceptibility to thyroid cancer.

In another aspect the invention further relates to a method for determining a susceptibility to thyroid cancer in a human individual, comprising determining whether at least one allele of at least one polymorphic marker is present in a nucleic acid sample obtained from the individual, or in a genotype dataset derived from the individual, wherein the at least one polymorphic marker is selected from rs965513 (SEQ ID NO:1), and markers in linkage disequilibrium therewith, and wherein the presence of the at least one allele is indicative of a susceptibility to thyroid cancer for the individual.

In another aspect, the invention relates to a method of determining a susceptibility to thyroid cancer in a human individual, comprising determining whether at least one at-risk allele in at least one polymorphic marker is present in a genotype dataset derived from the individual, wherein the at least one polymorphic marker is selected from markers rs965513 (SEQ ID NO:1), and markers in linkage disequilibrium therewith, and wherein determination of the presence of the at least one at-risk allele is indicative of increased susceptibility to thyroid cancer in the individual.

The genotype dataset comprises in one embodiment information about marker identity and the allelic status of the individual for at least one allele of a marker, i.e. information about the identity of at least one allele of the marker in the individual. The genotype dataset may comprise allelic information (information about allelic status) about one or more marker, including two or more markers, three or more markers, five or more markers, ten or more markers, one hundred or more markers, an so on. In some embodiments, the genotype dataset comprises genotype information from a whole-genome assessment of the individual, that may include hundreds of thousands of markers, or even one million or more markers spanning the entire genome of the individual.

In certain embodiments, the at least one polymorphic marker is associated with the FoxE1 gene.

Another aspect of the invention relates to a method of determining a susceptibility to thyroid cancer in a human individual, the method comprising:

obtaining nucleic acid sequence data about a human individual identifying at least one allele of at least one polymorphic marker selected from rs965513 (SEQ ID NO:1), and markers in linkage disequilibrium therewith, wherein different alleles of the at least one polymorphic marker are associated with different susceptibilities to thyroid cancer in humans, and
determining a susceptibility to thyroid cancer from the nucleic acid sequence data.

The invention also relates to a method of determining a susceptibility to thyroid cancer in a human individual, the method comprising obtaining nucleic acid sequence data about a human individual identifying at least one allele of at least one polymorphic marker associated with the FoxE1 gene, wherein different alleles of the at least one polymorphic marker are associated with different susceptibilities to thyroid cancer in humans, and determining a susceptibility to thyroid cancer from the nucleic acid sequence data.

In general, polymorphic genetic markers lead to alternate sequences at the nucleic acid level. If the nucleic acid marker changes the codon of a polypeptide encoded by the nucleic acid, then the marker will also result in alternate sequence at the amino acid level of the encoded polypeptide (polypeptide markers). Determination of the identity of particular alleles at polymorphic markers in a nucleic acid or particular alleles at polypeptide markers comprises whether particular alleles are present at a certain position in the sequence. Sequence data identifying a particular allele at a marker comprises sufficient sequence to detect the particular allele. For single nucleotide polymorphisms (SNPs) or amino acid polymorphisms described herein, sequence data can comprise sequence at a single position, i.e. the identity of a nucleotide or amino acid at a single position within a sequence. The sequence data can optionally include information about sequence flanking the polymorphic site, which in the case of SNPs spans a single nucleotide.

In certain embodiments, it may be useful to determine the nucleic acid sequence for at least two polymorphic markers. In other embodiments, the nucleic acid sequence for at least three, at least four or at least five or more polymorphic markers is determined. Haplotype information can be derived from an analysis of two or more polymorphic markers. Thus, in certain embodiments, a further step is performed, whereby haplotype information is derived based on sequence data for at least two polymorphic markers.

The invention also provides a method of determining a susceptibility to thyroid cancer in a human individual, the method comprising obtaining nucleic acid sequence data about a human individual identifying both alleles of at least two polymorphic markers selected from rs965513 (SEQ ID NO:1), and markers in linkage disequilibrium therewith, determine the identity of at least one haplotype based on the sequence data, and determine a susceptibility to thyroid cancer from the haplotype data.

In certain embodiments, determination of a susceptibility comprises comparing the nucleic acid sequence data to a database containing correlation data between the at least one polymorphic marker and susceptibility to thyroid cancer. In some embodiments, the database comprises at least one risk measure of susceptibility to thyroid cancer for the at least one marker. The sequence database can for example be provided as a look-up table that contains data that indicates the susceptibility of thyroid cancer for any one, or a plurality of, particular polymorphisms. The database may also contain data that indicates the susceptibility for a particular haplotype that comprises at least two polymorphic markers.

Obtaining nucleic acid sequence data can in certain embodiments comprise obtaining a biological sample from the human individual and analyzing sequence of the at least one polymorphic marker in nucleic acid in the sample. Analyzing sequence can comprise determining the presence or absence of at least one allele of the at least one polymorphic marker. Determination of the presence of a particular susceptibility allele (e.g., an at-risk allele) is indicative of susceptibility to thyroid cancer in the human individual. Determination of the absence of a particular suscepbility allele is indicative that the particular susceptibility due to the at least one polymorphism is not present in the individual.

In some embodiments, obtaining nucleic acid sequence data comprises obtaining nucleic acid sequence information from a preexisting record. The preexisting record can for example be a computer file or database containing sequence data, such as genotype data, for the human individual, for at least one polymorphic marker.

Susceptibility determined by the diagnostic methods of the invention can be reported to a particular entity. In some embodiments, the at least one entity is selected from the group consisting of the individual, a guardian of the individual, a genetic service provider, a physician, a medical organization, and a medical insurer.

In certain embodiments of the invention, determination of a susceptibility comprises comparing the nucleic acid sequence data to a database containing correlation data between the at least one polymorphic marker and susceptibility to thyroid cancer. In one such embodiment, the database comprises at least one risk measure of susceptibility to thyroid cancer for the at least one polymorphic marker. In another embodiment, the database comprises a look-up table containing at least one risk measure of the at least one condition for the at least one polymorphic marker.

In certain embodiments, obtaining nucleic acid sequence data comprises obtaining a biological sample from the human individual and analyzing sequence of the at least one polymorphic marker in nucleic acid in the sample. Analyzing sequence of the at least one polymorphic marker can comprise determining the presence or absence of at least one allele of the at least one polymorphic marker. Obtaining nucleic acid sequence data can also comprise obtaining nucleic acid sequence information from a preexisting record.

Certain embodiments of the invention relate to obtaining nucleic acid sequence data about at least two polymorphic markers selected from rs965513 (SEQ ID NO:1), and markers in linkage disequilibrium therewith.

In certain embodiments of the invention, the at least one polymorphic marker is selected from the markers set forth in Table 2. In one embodiment, the at least one polymorphic marker is selected from the markers as set forth in SEQ ID NO:1-229. In one embodiment, the at least one marker is in linkage disequilibrium with at least one of rs965513 (SEQ ID NO:1), rs907580 (SEQ ID NO:2) and rs7024345 (SEQ ID NO:3). In another embodiment, the at least one marker is in linkage disequilibrium with at least one marker selected from the group consisting of rs965513 (SEQ ID NO:1), rs10759944 (SEQ ID NO:17), rs907580 (SEQ ID NO:2), rs10984103 (SEQ ID NO:37), rs925487 (SEQ ID NO:34), rs7024345 (SEQ ID NO:3) and rs1443434 (SEQ ID NO:30). In one embodiment the at least one marker is selected from the group consisting of rs965513 (SEQ ID NO:1), rs10759944 (SEQ ID NO:17), rs907580 (SEQ ID NO:2), rs10984103 (SEQ ID NO:37), rs925487 (SEQ ID NO:34), rs7024345 (SEQ ID NO:3) and rs1443434 (SEQ ID NO:30).

In certain embodiments of the invention, a further step of assessing the frequency of at least one haplotype in the individual is performed. In such embodiments, two or more markers, including three, four, five, six, seven, eight, nine or ten or more markers can be included in the haplotype. In certain embodiments, the at least one haplotype comprises markers selected from the group consisting of rs965513 (SEQ ID NO:1), rs10759944 (SEQ ID NO:17), rs907580 (SEQ ID NO:2), rs10984103 (SEQ ID NO:37), rs925487 (SEQ ID NO:34), rs7024345 (SEQ ID NO:3) and rs1443434 (SEQ ID NO:30), and markers in linkage disequilibrium therewith. In certain such embodiments, the at least one haplotype is representative of the genomic structure of a particular genomic region (such as an LD block), to which any one of the above-mentioned markers reside.

The markers conferring risk of thyroid cancer, as described herein, can be combined with other genetic markers for thyroid cancer. Such markers are typically not in linkage disequilibrium with any one of the markers described herein, in particular markers rs965513 (SEQ ID NO:1), rs907580 (SEQ ID NO:2) and rs7024345 (SEQ ID NO:3) ID NO:6), rs9956546 (SEQ ID NO:7), rs11912922 (SEQ ID NO:8), rs6001954 (SEQ ID NO:9). Any of the methods described herein can be practiced by combining the genetic risk factors described herein with additional genetic risk factors for thyroid cancer.

Thus, in certain embodiments, a further step is included, comprising determining whether at least one at-risk allele of at least one at-risk variant for thyroid cancer not in linkage disequilibrium with any one of the markers rs965513 (SEQ ID NO:1), rs907580 (SEQ ID NO:2) and rs7024345 (SEQ ID NO:3) present in a sample comprising genomic DNA from a human individual or a genotype dataset derived from a human individual. In other words, genetic markers in other locations in the genome can be useful in combination with the markers of the present invention, so as to determine overall risk of thyroid cancer based on multiple genetic variants. In one embodiment, the at least one at-risk variant for thyroid cancer is not in linkage disequilibrium with marker rs965513 (SEQ ID NO:1). Selection of markers that are not in linkage disequilibrium (not in LD) can be based on a suitable measure for linkage disequilibrium, as described further herein. In certain embodiments, markers that are not in linkage disequilibrium have values for the LD measure r² correlating the markers of less than 0.2. In certain other embodiments, markers that are not in LD have values for r² correlating the markers of less than 0.15, including less than 0.10, less than 0.05, less than 0.02 and less than 0.01. Other suitable numerical values for establishing that markers are not in LD are contemplated, including values bridging any of the above-mentioned values.

In one embodiment, assessment of one or more of the markers described herein is combined with assessment of marker rs944289 on chromosome 14q13.3, or a marker in linkage disequilibrium therewith, is performed, to establish overall risk.

In certain embodiments, multiple markers as described herein are determined to determine overall risk of thyroid cancer. Thus, in certain embodiments, an additional step is included, the step comprising determining whether at least one allele in each of at least two polymorphic markers is present in a sample comprising genomic DNA from a human individual or a genotype dataset derived from a human individual, wherein the presence of the at least one allele in the at least two polymorphic markers is indicative of an increased susceptibility to thyroid cancer. In one embodiment, the markers are selected from the group consisting of rs965513 (SEQ ID NO:1), and markers in linkage disequilibrium therewith. In one embodiment, the markers are selected from the group consisting of the markers set forth in Table 2.

The genetic markers of the invention can also be combined with non-genetic information to establish overall risk for an individual. Thus, in certain embodiments, a further step is included, comprising analyzing non-genetic information to make risk assessment, diagnosis, or prognosis of the individual. The non-genetic information can be any information pertaining to the disease status of the individual or other information that can influence the estimate of overall risk of thyroid cancer for the individual. In one embodiment, the non-genetic information is selected from age, gender, ethnicity, socioeconomic status, previous disease diagnosis, medical history of subject, family history of thyroid cancer, biochemical measurements, and clinical measurements.

The invention also provides computer-implemented aspects. In one such aspect, the invention provides a computer-readable medium having computer executable instructions for determining susceptibility to thyroid cancer in an individual, the computer readable medium comprising: data representing at least one polymorphic marker; and a routine stored on the computer readable medium and adapted to be executed by a processor to determine susceptibility to thyroid cancer in an individual based on the allelic status of at least one allele of said at least one polymorphic marker in the individual.

In one embodiment, said data representing at least one polymorphic marker comprises at least one parameter indicative of the susceptibility to thyroid cancer linked to said at least one polymorphic marker. In another embodiment, said data representing at least one polymorphic marker comprises data indicative of the allelic status of at least one allele of said at least one allelic marker in said individual. In another embodiment, said routine is adapted to receive input data indicative of the allelic status for at least one allele of said at least one allelic marker in said individual. In a preferred embodiment, the at least one marker is selected from rs965513 (SEQ ID NO:1), and markers in linkage disequilibrium therewith. In another preferred embodiment, the at least one polymorphic marker is selected from the markers set forth in Table 2.

The invention further provides an apparatus for determining a genetic indicator for thyroid cancer in a human individual, comprising:

a processor,
a computer readable memory having computer executable instructions adapted to be executed on the processor to analyze marker and/or haplotype information for at least one human individual with respect to thyroid cancer, and
generate an output based on the marker or haplotype information, wherein the output comprises a risk measure of the at least one marker or haplotype as a genetic indicator of thyroid cancer for the human individual. In one embodiment, the computer readable memory comprises data indicative of the frequency of at least one allele of at least one polymorphic marker or at least one haplotype in a plurality of individuals diagnosed with thyroid cancer, and data indicative of the frequency of at the least one allele of at least one polymorphic marker or at least one haplotype in a plurality of reference individuals, and wherein a risk measure is based on a comparison of the at least one marker and/or haplotype status for the human individual to the data indicative of the frequency of the at least one marker and/or haplotype information for the plurality of individuals diagnosed with thyroid cancer. In one embodiment, the computer readable memory further comprises data indicative of a risk of developing thyroid cancer associated with at least one allele of at least one polymorphic marker or at least one haplotype, and wherein a risk measure for the human individual is based on a comparison of the at least one marker and/or haplotype status for the human individual to the risk associated with the at least one allele of the at least one polymorphic marker or the at least one haplotype. In another embodiment, the computer readable memory further comprises data indicative of the frequency of at least one allele of at least one polymorphic marker or at least one haplotype in a plurality of individuals diagnosed with thyroid cancer, and data indicative of the frequency of at the least one allele of at least one polymorphic marker or at least one haplotype in a plurality of reference individuals, and wherein risk of developing thyroid cancer is based on a comparison of the frequency of the at least one allele or haplotype in individuals diagnosed with thyroid cancer, and reference individuals. In a preferred embodiment, the at least one marker is selected from the group consisting of rs965513 (SEQ ID NO:1), and markers in linkage disequilibrium therewith. In another preferred embodiment, the at least one polymorphic marker is selected from the group consisting of the markers set forth in Table 2.

In another aspect, the invention relates to a method of identification of a marker for use in assessing susceptibility to thyroid cancer, the method comprising: identifying at least one polymorphic marker in linkage disequilibrium with at least one of rs965513 (SEQ ID NO:1), rs907580 (SEQ ID NO:2) and rs7024345 (SEQ ID NO:3); determining the genotype status of a sample of individuals diagnosed with, or having a susceptibility to, thyroid cancer; and determining the genotype status of a sample of control individuals; wherein a significant difference in frequency of at least one allele in at least one polymorphism in individuals diagnosed with, or having a susceptibility to, thyroid cancer, as compared with the frequency of the at least one allele in the control sample is indicative of the at least one polymorphism being useful for assessing susceptibility to thyroid cancer. Significant difference can be estimated on statistical analysis of allelic counts at certain polymorphic markers in thyroid cancer patients and controls. In one embodiment, a significant difference is based on a calculated P-value between thyroid cancer patients and controls of less than 0.05. In other embodiments, a significant difference is based on a lower value of the calculated P-value, such as less than 0.005, 0.0005, or less than 0.00005. In one embodiment, an increase in frequency of the at least one allele in the at least one polymorphism in individuals diagnosed with, or having a susceptibility to, thyroid cancer, as compared with the frequency of the at least one allele in the control sample is indicative of the at least one polymorphism being useful for assessing increased susceptibility to thyroid cancer. In another embodiment, a decrease in frequency of the at least one allele in the at least one polymorphism in individuals diagnosed with, or having a susceptibility to, thyroid cancer, as compared with the frequency of the at least one allele in the control sample is indicative of the at least one polymorphism being useful for assessing decreased susceptibility to, or protection against, thyroid cancer.

The invention also relates to a method of genotyping a nucleic acid sample obtained from a human individual comprising determining whether at least one allele of at least one polymorphic marker is present in a nucleic acid sample from the individual sample, wherein the at least one marker is selected from the group consisting of rs965513 (SEQ ID NO:1), and markers in linkage disequilibrium therewith, and wherein determination of the presence of the at least one allele in the sample is indicative of a susceptibility to thyroid cancer in the individual. In one embodiment, determination of the presence of allele C of rs965513 (SEQ ID NO:1) is indicative of increased susceptibility of thyroid cancer in the individual. In one embodiment, genotyping comprises amplifying a segment of a nucleic acid that comprises the at least one polymorphic marker by Polymerase Chain Reaction (PCR), using a nucleotide primer pair flanking the at least one polymorphic marker. In another embodiment, genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, single-stranded conformation analysis and microarray technology. In one embodiment, the microarray technology is Molecular Inversion Probe array technology or BeadArray Technologies. In one embodiment, the process comprises allele-specific probe hybridization. In another embodiment, the process comprises microrray technology. One preferred embodiment comprises the steps of (1) contacting copies of the nucleic acid with a detection oligonucleotide probe and an enhancer oligonucleotide probe under conditions for specific hybridization of the oligonucleotide probe with the nucleic acid; wherein (a) the detection oligonucleotide probe is from 5-100 nucleotides in length and specifically hybridizes to a first segment of a nucleic acid whose nucleotide sequence is given by any one of SEQ ID NO:1-229; (b) the detection oligonucleotide probe comprises a detectable label at its 3′ terminus and a quenching moiety at its 5′ terminus; (c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and (d) a single base gap exists between the first segment and the second segment, such that when the oligonucleotide probe and the enhancer oligonucleotide probe are both hybridized to the nucleic acid, a single base gap exists between the oligonucleotides; (2) treating the nucleic acid with an endonuclease that will cleave the detectable label from the 3′ terminus of the detection probe to release free detectable label when the detection probe is hybridized to the nucleic acid; and (3) measuring free detectable label, wherein the presence of the free detectable label indicates that the detection probe specifically hybridizes to the first segment of the nucleic acid, and indicates the sequence of the polymorphic site as the complement of the detection probe.

A further aspect of the invention pertains to a method of assessing an individual for probability of response to a thyroid cancer therapeutic agent, comprising: determining whether at least one allele of at least one polymorphic marker is present in a nucleic acid sample obtained from the individual, or in a genotype dataset derived from the individual, wherein the at least one polymorphic marker is selected from the group consisting of rs965513 (SEQ ID NO:1), and markers in linkage disequilibrium therewith, wherein the presence of the at least one allele of the at least one marker is indicative of a probability of a positive response to the therapeutic agent.

The invention in another aspect relates to a method of predicting prognosis of an individual diagnosed with thyroid cancer, the method comprising determining whether at least one allele of at least one polymorphic marker is present in a nucleic acid sample obtained from the individual, or in a genotype dataset derived from the individual, wherein the at least one polymorphic marker is selected from the group consisting of rs965513 (SEQ ID NO:1), and markers in linkage disequilibrium therewith, wherein the presence of the at least one allele is indicative of a worse prognosis of the thyroid cancer in the individual.

Yet another aspect of the invention relates to a method of monitoring progress of treatment of an individual undergoing treatment for thyroid cancer, the method comprising determining whether at least one allele of at least one polymorphic marker is present in a nucleic acid sample obtained from the individual, or in a genotype dataset derived from the individual, wherein the at least one polymorphic marker is selected from the group consisting of rs965513 (SEQ ID NO:1), and markers in linkage disequilibrium therewith, wherein the presence of the at least one allele is indicative of the treatment outcome of the individual. In one embodiment, the treatment is treatment by surgery, treatment by radiation therapy, or treatment by drug administration.

The invention also relates to the use of an oligonucleotide probe in the manufacture of a reagent for diagnosing and/or assessing susceptibility to thyroid cancer in a human individual, wherein the probe hybridizes to a segment of a nucleic acid with nucleotide sequence as set forth in any one of SEQ ID NO:1-229, wherein the probe is 15-500 nucleotides in length. In certain embodiments, the probe is about 16 to about 100 nucleotides in length. In certain other embodiments, the probe is about 20 to about 50 nucleotides in length. In certain other embodiments, the probe is about 20 to about 30 nucleotides in length.

The present invention, in its broadest sense relates to any subphenotype of thyroid cancer, including papillary, fillicular, medullary and anaplastic thyroid cancer. In certain embodiments, the invention relates to certain tumor types. Thus, in one embodiment, the invention relates to papillary thyroid cancer. In another embodiment, the invention relates to follicular thyroid cancer. In another embodiment, the invention relates to papillary and/or follicular thyroid cancer. In another embodiment, the invention relates to medullary thyroid cancer. In yet another embodiment, the invention relates to anaplastic thyroid cancer. Other subphenotypes of thyroid cancer, as well as other combinations of subphenotypes are also contemplated and are also within scope of the present invention.

Certain embodiments of the invention relate to diagnosis of thyroid cancer with an early age at onset and/or an early age at diagnosis. Thyroid cancer diagnosed at an early age may be more aggressive, in particular when benign nodules are present at an early age. Thus, certain embodiments relate to thyroid cancer occurring with an early age at onset and/or an early age of diagnosis.

Certain embodiments of the invention further comprise assessing the quantitative levels of a biomarker for thyroid cancer. The biomarker may in some embodiments be assessed in a biological sample from the individual. In some embodiments, the sample is a blood sample. The blood sample is in some embodiments a serum sample. In preferred embodiments, the biomarker is selected from the group consisting of thyroid stimulating hormone (TSH), thyroxine (T4) and thriiodothyronine (T3). In certain embodiments, determination of an abnormal level of the biomarker is indicative of an abnormal thyroid function in the individual, which may in turn be indicative of an increased risk of thyroid cancer in the individual. The abnormal level can be an increased level or the abnormal level can be a decreased level. In certain embodiments, the determination of an abnormal level is determined based on determination of a deviation from the average levels of the biomarke in the population. In one embodiment, abnormal levels of TSH are measurements of less than 0.2 mIU/L and/or greater than 10 mIU/L. In another embodiment, abnormal levels of TSH are measurements of less than 0.3 mIU/L and/or greater than 3.0 mIU/L. In another embodiment, abnormal levels of T₃ (free T₃) are less than 70 ng/dL and/or greater than 205 ng/dL. In another embodiment, abnormal levels of T₄ (free T₄) are less than 0.8 ng/dL and/or greater than 2.7 ng/dL.

In some embodiments of the methods of the invention, the susceptibility determined in the method is increased susceptibility. In one such embodiment, the increased susceptibility is characterized by a relative risk (RR) or an odds ratio (OR) of at least 1.30. In another embodiment, the increased susceptibility is characterized by a relative risk or an odds ratio of at least 1.40. In another embodiment, the increased susceptibility is characterized by a relative risk or an odds ratio of at least 1.50. In another embodiment, the increased susceptibility is characterized by a relative risk or an odds ratio of at least 1.60. In yet another embodiment, the increased susceptibility is characterized by a relative risk or an odds ratio of at least 1.70. In a further embodiment, the increased susceptibility is characterized by a relative risk or an odds ratio of at least 1.80. In a further embodiment, the increased susceptibility is characterized by a relative risk or an odds ratio of at least 1.90. In yet another embodiment, the increased susceptibility is characterized by a relative risk or an odds ratio of at least 2.0. Cerain other embodiments are characterized by relative risk or an odds ratio of the at-risk variant of at least 1.55, 1.65, 1.75, 1.85 and 1.95. Other numeric values of relative risks and/or odds ratios, including those bridging any of these above-mentioned values are also possible, and these are also within scope of the invention.

In some embodiments of the methods of the invention, the susceptibility determined in the method is decreased susceptibility. In one such embodiment, the decreased susceptibility is characterized by a relative risk (RR) or an odds ratio (OR) of less than 0.8. In another embodiment, the decreased susceptibility is characterized by a relative risk or an odds ratio of less than 0.7. In another embodiment, the decreased susceptibility is characterized by a relative risk or an odds ratio of less than 0.6. In yet another embodiment, the decreased susceptibility is characterized by a relative risk or an odds ratio of less than 0.5. Other cutoffs, such as relative risk or an odds ratio of less than 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50, and so on, are also contemplated and are within scope of the invention.

The invention also relates to kits. In one such aspect, the invention relates to a kit for assessing susceptibility to thyroid cancer in a human individual, the kit comprising reagents necessary for selectively detecting at least one allele of at least one polymorphic marker selected from the group consisting of rs965513 (SEQ ID NO:1), and markers in linkage disequilibrium therewith, in the genome of the individual, wherein the presence of the at least one allele is indicative of increased susceptibility to thyroid cancer. In another aspect, the invention relates to a kit for assessing susceptibility to thyroid cancer in a human individual, the kit comprising reagents for selectively detecting at least one allele of at least one polymorphic marker in the genome of the individual, wherein the polymorphic marker is selected from the group consisting of rs965513 (SEQ ID NO:1), and wherein the presence of the at least one allele is indicative of a susceptibility to thyroid cancer. In one embodiment, the at least one polymorphic marker is selected from the markers set forth in Table 2.

Kit reagents may in one embodiment comprise at least one contiguous oligonucleotide that hybridizes to a fragment of the genome of the individual comprising the at least one polymorphic marker. In another embodiment, the kit comprises at least one pair of oligonucleotides that hybridize to opposite strands of a genomic segment obtained from the subject, wherein each oligonucleotide primer pair is designed to selectively amplify a fragment of the genome of the individual that includes one polymorphism, wherein the polymorphism is selected from the group consisting of the polymorphisms as defined in Table 2, and wherein the fragment is at least 20 base pairs in size. In one embodiment, the oligonucleotide is completely complementary to the genome of the individual. In another embodiment, the kit further contains buffer and enzyme for amplifying said segment. In another embodiment, the reagents further comprise a label for detecting said fragment.

In one preferred embodiment, the kit comprises: a detection oligonucleotide probe that is from 5-100 nucleotides in length; an enhancer oligonucleotide probe that is from 5-100 nucleotides in length; and an endonuclease enzyme; wherein the detection oligonucleotide probe specifically hybridizes to a first segment of the nucleic acid whose nucleotide sequence is set forth in any one of SEQ ID NO:1-229, and wherein the detection oligonucleotide probe comprises a detectable label at its 3′ terminus and a quenching moiety at its 5′ terminus; wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; wherein a single base gap exists between the first segment and the second segment, such that when the oligonucleotide probe and the enhancer oligonucleotide probe are both hybridized to the nucleic acid, a single base gap exists between the oligonucleotides; and wherein treating the nucleic acid with the endonuclease will cleave the detectable label from the 3′ terminus of the detection probe to release free detectable label when the detection probe is hybridized to the nucleic acid.

Kits according to the present invention may also be used in the other methods of the invention, including methods of assessing risk of developing at least a second primary tumor in an individual previously diagnosed with thyroid cancer, methods of assessing an individual for probability of response to a thyroid cancer therapeutic agent, and methods of monitoring progress of a treatment of an individual diagnosed with thyroid cancer and given a treatment for the disease.

In certain embodiments of the methods, uses, apparatus or kits of the invention, the at least one polymorphic marker that provides information about susceptibility to thyroid cancer is associated with the FoxE1 gene. Being “associated with”, in this context, means that the at least one marker is in linkage disequilibrium with the FoxE1 gene or its regulatory regions. Such markers can be located within the FoxE1 gene, or its regulatory regions, or they can be in linkage disequilibrium with at least one marker within the FoxE1 gene or its regulatory region that has a direct impact on the function of the gene. The functional consequence of the susceptibility variants associated with the FoxE1 can be on the expression level of the FoxE1 gene, the stability of its transcript or through amino acid alterations at the protein level, as described in more detail herein.

The markers that are described herein to be associated with thyroid cancer can all be used in the various aspects of the invention, including the methods, kits, uses, apparatus, procedures described herein. In certain embodiments, the invention relates to markers associated with the C09 LD Block as defined herein. In certain other embodiments, the invention relates to the markers set forth in Table 2 (SEQ ID NO:1-229), and markers in linkage disequilibrium therewith. In certain other embodiments, the invention relates to the markers set forth in Table 2. In certain other embodiments, the invention relates to markers rs965513 (SEQ ID NO:1), rs10759944 (SEQ ID NO:17), rs907580 (SEQ ID NO:2), rs10984103 (SEQ ID NO:37), rs925487 (SEQ ID NO:34), rs7024345 (SEQ ID NO:3) and rs1443434 (SEQ ID NO:30), and markers in linkage disequilibrium therewith. In some other preferred embodiments, the invention relates to any one of the markers selected from the group consisting of rs965513 (SEQ ID NO:1), rs10759944 (SEQ ID NO:17), rs907580 (SEQ ID NO:2), rs10984103 (SEQ ID NO:37), rs925487 (SEQ ID NO:34), rs7024345 (SEQ ID NO:3) and rs1443434 (SEQ ID NO:30).

In certain embodiments, the at least one marker allele conferring increased risk of thyroid cancer is selected from the group consisting of rs965513 allele A, rs10759944 allele A, rs907580 allele A, rs10984103 allele A, rs925487 allele G, rs7024345 allele A and rs1443434 allele G. In these embodiments, the presence of the allele (the at-risk allele) is indicative of increased risk of thyroid cancer.

In certain embodiments of the invention, linkage disequilibrium is determined using the linkage disequilibrium measures r² and |D′|, which give a quantitative measure of the extent of linkage disequilibrium (LD) between two genetic element (e.g., polymorphic markers). Certain numerical values of these measures between particular markers are indicative of the markers being in linkage disequilibrium, as described further herein. In one embodiment of the invention, linkage disequilibrium between markers (i.e., LD values indicative of the markers being in linkage disequilibrium) is defined as r²>0.1. In another embodiment, linkage disequilibrium is defined as r²>0.2. Other embodiments can include other definitions of linkage disequilibrium, such as r²>0.25, r²>0.3, r²>0.35, r²>0.4, r²>0.45, r²>0.5, r²>0.55, r²>0.6, r²>0.65, r²>0.7, r²>0.75, r²>0.8, r²>0.85, r²>0.9, r²>0.95, r²>0.96, r²>0.97, r²>0.98, or r²>0.99. Linkage disequilibrium can in certain embodiments also be defined as |D′|>0.2, or as |D′|>0.3, |D′|>0.4, |D′|>0.5, |D′|>0.6, |D′|>0.7, |D′|>0.8, |D′|>0.9, |D′|>0.95, |D′|>0.98 or |D′|>0.99. In certain embodiments, linkage disequilibrium is defined as fulfilling two criteria of r² and |D′|, such as r²>0.2 and |D′|>0.8. Other combinations of values for r² and |D′| are also possible and within scope of the present invention, including but not limited to the values for these parameters set forth in the above.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 shows a schematic view of the association results and LD-structure in a region on chromosome 9q22.33. (a) Single marker (diamonds) association results for SNPs from the Illumine Hap300/370 chip. Shown are P values corrected for relatedness. (b) Pair-wise correlation coefficient (r²) from the CEU HapMap population and the relative location of genes in the region, based on the UCSC Genome Browser, Build 36.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-O₂) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein, including sequence listing, is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “3 rs965513” refers to the 3 allele of marker rs7758851 being in the haplotype, and is equivalent to “rs965513 allele 3”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of thyroid cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of thyroid cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or acess of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “thyroid cancer therapeutic agent” refers to an agent that can be used to ameliorate or prevent symptoms associated with thyroid cancer.

The term “thyroid cancer-associated nucleic acid”, as described herein, refers to a nucleic acid that has been found to be associated to thyroid cancer. This includes, but is not limited to, the markers and haplotypes described herein and markers and haplotypes in strong linkage disequilibrium (LD) therewith. In one embodiment, a thyroid cancer-associated nucleic acid refers to a genomic region, such as an LD-block, found to be associated with risk of thyroid cancer through at least one polymorphic marker located within the region or LD block.

The term “FoxE1” or “FoxE1 gene”, as described herein, refers to the Forkhead Factor E1 gene formerly called thyroid transcription factor 2 (TTF-2) on chromosome 9q22.33.

The term “LD Block C09”, as described herein, refers to the Linkage Disequilibrium (LD) block region on Chromosome 9 that spans markers rs2795492 and rs7855669, corresponding to position 99,350,532-99,953,197 of NCBI (National Center for Biotechnology Information) Build 36 (SEQ ID NO:1).

Through a genome-wide search for genetic variants that confer susceptibility to thyroid cancer, the present inventors have identified a region on chromosome 9q22.33 that contains variants that associate with risk of thyroid cancer. Markers rs965513, rs907580 and rs7024345 were found to be significantly associated with risk of thyroid cancer. The strongest association signal was observed for marker rs965513 (OR1.77, P-value 1.18×10⁻¹⁵). Follow-up analysis confirmed this result, both in Iceland and in samples from the United States and Spain (overall P-value 1.7×10⁻²² for rs965513).

The rs965513 marker is located within a region on chromosome 9q22.33 characterized by extensive linkage disequilibrium. The consequence of such extensive LD is that a number of genetic variants within the region are surrogates for the at-risk variant rs965513, including for example rs907580 and rs7024345, and also rs10759944, rs10984103, rs925487 and rs1443434), and such markers are also useful for realizing the present invention. Other SNP markers useful for realizing the invention due to being in LD with rs965513 are provided in Table 2 herein. As discussed in more detail in the below, surrogate markers can extend over a large genomic region, depending on the genomic structure of the region. For example, the surrogate markers for rs965513 set forth in Table 2 herein span a region of approximately 600 kb (also called LD Block C09 herein). Functional units that are responsible for the biological consequence of the genetic risk for thyroid cancer identified in this region can in principle be located anywhere within the region of extensive LD. Markers that are in particularly high LD with rs965513 (e.g., LD characterized by high values for r² and/or D′, as described further in the below, e.g. r² values greater than 0.1 or 0.2) are most likely to be within, or in high LD with, such units.

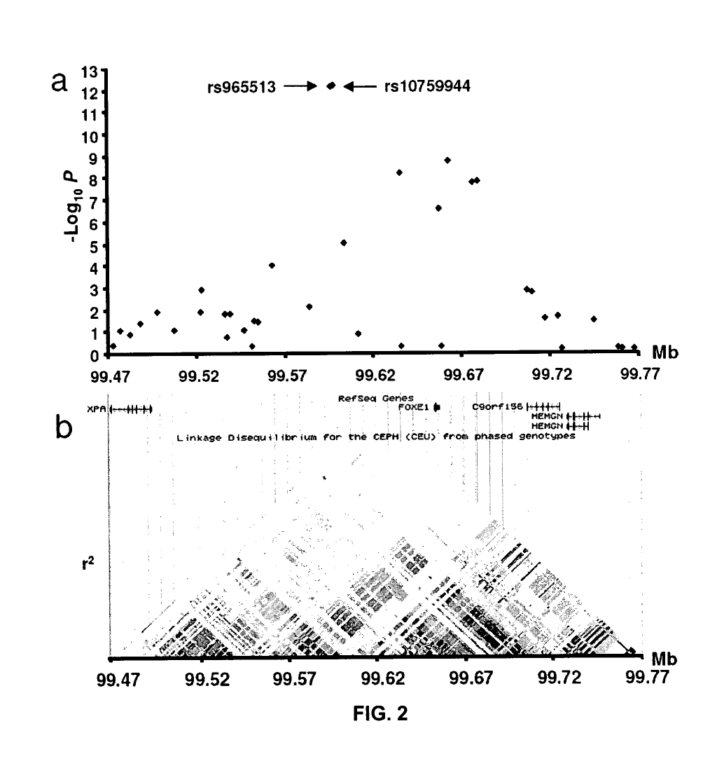

The Forkhead factor E1 (FoxE1; formerly called thyroid transcription factor 2 (TTF-2)) gene is located near rs965513, and within the region containing markers in strong LD with rs965513. Other genes in the region include XPA, C9orf156 and HEMGN (FIG. 2) The FoxE1 gene regulates the expression of thyroid-specific genes (De Felice, M., and R. Di Lauro., Endocr. Rev. 25:722-746 (2004); Francis-Lang, H., et al., Mol. Cell. Biol. 12:576-588 (1992); Sinclair, A. et al. Eur. J. Biochem. 193:311-318 (1990)), and it is essential for thyroid gland formation (Dathan, N., R. Parlato, A. Rosica, M. De Felice, and R. Di Lauro, Dev. Dyn. 224:450-456 (2002)) and migration (De Felice, M., et al. Nat. Genet. 19:395-398 (1998)), being at the center of a regulatory network of transcription factors and cofactors that initiate thyroid differentiation (Parlato, R., et al. Dev. Biol. 276:464-475 (2004)). Mutations of the FoxE1 gene cause human syndromes that are associated with thyroid agenesis, among other phenotypes (Castanet, M., et al., Hum Mol Genet. 11:2051-9 (2002); Clifton-Bligh, R. J., et al. Nat. Genet. 19:399-401 (1998)). FoxE1 is also necessary for the maintenance of the thyroid differentiated state, because it is essential for the hormonal control of the transcription of thyroid-specific genes, such as the thyroglobulin (Tg) (Santisteban, P., et al., Mol. Endocrinol. 6:1310-1317 (1992)) and thyroperoxidase (TPO) (Aza-Blanc, P., R. Di Lauro, and P. Santisteban. Mol. Endocrinol. 7:1297-1306 (1993)) genes. TPO gene expression is also regulated by TTF-1 (Nkx2.1), Pax8, and nuclear factor 1 (NF-1). Among these factors, FoxE1 is the main mediator of TPO response to thyroid-stimulating hormone (TSH) and insulin-like growth factor 1 (IGF-1) (Aza-Blanc, P., R. Di Laura, and P. Santisteban. Mol. Endocrinol. 7:1297-1306 (1993)). The expression of FoxE1, as well as its DNA binding and transcriptional activity, is activated by TSH and IGF-1, with the FoxE1 DNA binding site constituting a hormone response element that regulates the specific expression of thyroid genes (Ortiz, L., et al. J. Biol. Chem. 272:23334-23339 (1997)). FOXE1 is also necessary for the maintenance of the differentiated state of the thyroid, based on its involvement in regulating the transcription of thyroid-specific genes, such as the thyroglobulin (Tg) and thyroperoxidase (TPO) genes. Regulated expression of both of these genes is pivotal for the synthesis of the thyroid hormones triiodothyronine (T₃) and thyroxine (T₄) as Tg is the precursor of the T₃ and T₄, and their synthesis is catalyses by TPO. Central to the thyroid hormone synthesis and secretion control is the thyroid stimulating hormone (TSH) that acts as principal regulator.

The present inventors have also found that rs965513 associates with levels of TSH, free T₄ and free T₃ in serum, further confirming the association of markers in the chromosome 9q22 region with thyroid cancer and thyroid cancer-related biological activity.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome. For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including mini- and microsatellites, and insertions, deletions and inversions (also called copy number variations (CNVs)). A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

Due to their abundance, SNPs account for a majority of sequence variation in the human genome. Over 6 million SNPs have been validated to date (http://www.ncbi.nlm.nih.gov/projects/SNP/snp_summary.cgi). However, CNVs are receiving increased attention. These large-scale polymorphisms (typically 1 kb or larger) account for polymorphic variation affecting a substantial proportion of the assembled human genome; known CNVs covery over 15% of the human genome sequence (Estivill, X Armengol; L., PloS Genetics 3:1787-99 (2007). A http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the markers described herein to be associated with thyroid cancer. Methods for detecting CNVs include comparative genomic hybridization (CGH) and genotyping, including use of genotyping arrays, as described by Carter (Nature Genetics 39:S16-S21 (2007)). The Database of Genomic Variants (http://projects.tcag.ca/variation/) contains updated information about the location, type and size of described CNVs. The database currently contains data for over 15,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the complimentary strand on the DNA template, the presence of the complementary bases T and C can be measured. Quantitatively (for example, in terms of relative risk), identical results would be obtained from measurement of either DNA strand (+ strand or − strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNP5 that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

In the present context, and individual who is at an increased susceptibility (i.e., increased risk) for a disease, is an individual in whom at least one specific allele at one or more polymorphic marker or haplotype conferring increased susceptibility (increased risk) for the disease is identified (i.e., at-risk marker alleles or haplotypes). The at-risk marker or haplotype is one that confers an increased risk (increased susceptibility) of the disease. In one embodiment, significance associated with a marker or haplotype is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least 1.5 is significant. In another further embodiment, a significant increase in risk is at least 1.7 is significant. However, other cutoffs are also contemplated, e.g., at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype as described herein is one where at least one allele of at least one marker or haplotype is more frequently present in an individual at risk for the disease (or trait) (affected), or diagnosed with the disease, compared to the frequency of its presence in a comparison group (control), such that the presence of the marker or haplotype is indicative of susceptibility to the disease. The control group may in one embodiment be a population sample, i.e. a random sample from the general population. In another embodiment, the control group is represented by a group of individuals who are disease-free. Such disease-free controls may in one embodiment be characterized by the absence of one or more specific disease-associated symptoms. Alternatively, the disease-free controls are those that have not been diagnosed with the disease. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors comprise at least one additional genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

In other embodiments of the invention, an individual who is at a decreased susceptibility (i.e., at a decreased risk) for a disease or trait is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk (or odds ratio) of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNP5), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

A genetic variant associated with a disease or a trait can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations for a plurality of risk variants usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (Gudmundsson, 3., et al., Nat Genet. 39:631-7 (2007), Gudmundsson, 3., et al., Nat Genet 39:977-83 (2007); Yeager, M., et al, Nat Genet 39:645-49 (2007), Amundadottir, L., et al., Nat Genet 38:652-8 (2006); Haiman, C. A., et al., Nat Genet 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with a plurality of variants associated with thyroid cancer may be assessed, including combinations of any one of the markers rs965513 (SEQ ID NO:1), rs907580 (SEQ ID NO:2) and rs7024345 (SEQ ID NO:3), or markers in linkage disequilibrium therewith.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurence of each allele or haplotype in the population. For populations of diploids, e.g., human populations, individuals will typically have two alleles or allelic combinations for each genetic element (e.g., a marker, haplotype or gene).

Many different measures have been proposed for assessing the strength of linkage disequilibrium (LD; reviewed in Devlin, B. & Risch, N., Genomics 29:311-22 (1995))). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, or at least 0.99. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium is defined as r²>0.2 and |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian, african, japanese, chinese), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with the variants described herein may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (such as <10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. 8, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a susceptibility region, for example within an LD block, association of all possible combinations of genotyped markers within the region is studied. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families to the study, first and second-degree relatives can be eliminated from the patient list. Furthermore, the test can be repeated for association correcting for any remaining relatedness among the patients, by extending a variance adjustment procedure previously described (Risch, N. & Teng, J. Genome Res., 8:1273-1288 (1998)) for sibships so that it can be applied to general familial relationships, and present both adjusted and unadjusted p-values for comparison. The method of genomic controls (Devlin, B. & Roeder, K. Biometrics 55:997 (1999)) can also be used to adjust for the relatedness of the individuals and possible stratification. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, ideally from a different population (e.g., different region of same country, or a different country) of the same or different ethnicity. The advantage of replication studies is that the number of tests performed in the replication study, and hence the less stringent the statistical measure that is applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold are a measure of a true genetic effect, and replication in additional cohorts is not necessarily from a statistical point of view. However, since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds-ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds-ratio.

It is well known that under the rare disease assumption, the relative risk of a disease can be approximated by the odds-ratio. This assumption may however not hold for many common diseases. Still, it turns out that the risk of one genotype variant relative to another can be estimated from the odds-ratio expressed above. The calculation is particularly simple under the assumption of random population controls where the controls are random samples from the same population as the cases, including affected people rather than being strictly unaffected individuals. To increase sample size and power, many of the large genome-wide association and replication studies used controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds-ratio of individuals is the same as the risk-ratio between these variants:

OR=Pr(A|c)/Pr(A|nc)=r

And likewise for the multiplicative model, where the risk is the product of the risk associated with the two allele copies, the allelic odds-ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### The Risk Relative to the Average Population Risk

It is most convenient to represent the risk of a genetic variant relative to the average population since it makes it easier to communicate the lifetime risk for developing the disease compared with the baseline population risk. For example, in the multiplicative model we can calculate the relative population risk for variant “aa” as:

RR(aa)=Pr(A|aa)/Pr(A)=(Pr(A|aa)/Pr(A|bb))/(Pr(A)/Pr(A|bb))=r²/(Pr(aa)r²+Pr(ab)r+Pr(bb))=r²/(p²r²+2pqr+q²)=r²/R

Here “p” and “q” are the allele frequencies of “a” and “b” respectively. Likewise, we get that RR(ab)=r/R and RR(bb)=1/R. The allele frequency estimates may be obtained from the publications that report the odds-ratios and from the HapMap database. Note that in the case where we do not know the genotypes of an individual, the relative genetic risk for that test or marker is simply equal to one.

As an example, in type-2 diabetes risk, allele T of the disease associated marker rs7903146 in the TCF7L2 gene on chromosome 10 has an allelic OR of 1.37 and a frequency (p) around 0.28 in non-Hispanic white populations. The genotype relative risk compared to genotype CC are estimated based on the multiplicative model.

For TT it is 1.37×1.37=1.88; for CT it is simply the OR 1.37, and for CC it is 1.0 by definition.

The frequency of allele C is q=1−p=1−0.28=0.72. Population frequency of each of the three possible genotypes at this marker is:

Pr(TT)=p²=0.08, Pr(CT)=2pq=0.40, and Pr(CC)=q²=0.52

The average population risk relative to genotype CC (which is defined to have a risk of one) is:

R=0.08×1.88+0.40×1.37+0.52×1=1.22

Therefore, the risk relative to the general population (RR) for individuals who have one of the following genotypes at this marker is:

RR(TT)=1.88/1.22=1.54, RR(CT)=1.37/1.22=1.12, RR(CC)=1/1.22=0.82.

Combining the Risk from Multiple Markers

When genotypes of many SNP variants are used to estimate the risk for an individual, unless otherwise stated, a multiplicative model for risk can be assumed. This means that the combined genetic risk relative to the population is calculated as the product of the corresponding estimates for individual markers, e.g. for two markers g1 and g2:

RR(g1,g2)=RR(g1)RR(g2)

The underlying assumption is that the risk factors occur and behave independently, i.e. that the joint conditional probabilities can be represented as products:

Pr(A|g1,g2)=Pr(A|g1)Pr(A|g2)/Pr(A) and Pr(g1,g2)=Pr(g1)Pr(g2)

Obvious violations to this assumption are markers that are closely spaced on the genome, i.e. in linkage disequilibrium such that the concurrence of two or more risk alleles is correlated. In such cases, we can use so called haplotype modeling where the odds-ratios are defined for all allele combinations of the correlated SNPs.

As is in most situations where a statistical model is utilized, the model applied is not expected to be exactly true since it is not based on an underlying bio-physical model. However, the multiplicative model has so far been found to fit the data adequately, i.e. no significant deviations are detected for many common diseases for which many risk variants have been discovered.

As an example, an individual who has the following genotypes at 4 markers associated with risk of type-2 diabetes along with the risk relative to the population at each marker:

Chromo 3 PPARG CC Calculated risk: RR(CC)=1.03
Chromo 6 CDKAL1 GG Calculated risk: RR(GG)=1.30
Chromo 9 CDKN2A AG Calculated risk: RR(AG)=0.88
Chromo 11 TCF7L2 TT Calculated risk: RR(TT)=1.54

Combined, the overall risk relative to the population for this individual is: 1.03×1.30×0.88×1.54=1.81

### Adjusted Life-Time Risk

The lifetime risk of an individual is derived by multiplying the overall genetic risk relative to the population with the average life-time risk of the disease in the general population of the same ethnicity and gender and in the region of the individual's geographical origin. As there are usually several epidemiologic studies to choose from when defining the general population risk, we will pick studies that are well-powered for the disease definition that has been used for the genetic variants.

For example, for type-2 diabetes, if the overall genetic risk relative to the population is 1.8 for a white male, and if the average life-time risk of type-2 diabetes for individuals of his demographic is 20%, then the adjusted lifetime risk for him is 20%×1.8=36%.

Note that since the average RR for a population is one, this multiplication model provides the same average adjusted life-time risk of the disease. Furthermore, since the actual life-time risk cannot exceed 100%, there must be an upper limit to the genetic RR.

### Risk Assessment for Thyroid Cancer

As described herein, certain polymorphic markers and haplotypes comprising such markers are found to be useful for risk assessment of thyroid cancer. Risk assessment can involve the use of the markers for determining a susceptibility to thyroid cancer. Particular alleles of polymorphic markers (e.g., SNPs) are found more frequently in individuals with thyroid cancer, than in individuals without diagnosis of thyroid cancer. Therefore, these marker alleles have predictive value for detecting thyroid cancer, or a susceptibility to thyroid cancer, in an individual. Tagging markers in linkage disequilibrium with at-risk variants (or protective variants) described herein can be used as surrogates for these markers (and/or haplotypes). Such surrogate markers can be located within a particular haplotype block or LD block. Such surrogate markers can also sometimes be located outside the physical boundaries of such a haplotype block or LD block, either in close vicinity of the LD block/haplotype block, but possibly also located in a more distant genomic location.

Long-distance LD can for example arise if particular genomic regions (e.g., genes) are in a functional relationship. For example, if two genes encode proteins that play a role in a shared metabolic pathway, then particular variants in one gene may have a direct impact on observed variants for the other gene. Let us consider the case where a variant in one gene leads to increased expression of the gene product. To counteract this effect and preserve overall flux of the particular pathway, this variant may have led to selection of one (or more) variants at a second gene that conferes decreased expression levels of that gene. These two genes may be located in different genomic locations, possibly on different chromosomes, but variants within the genes are in apparent LD, not because of their shared physical location within a region of high LD, but rather due to evolutionary forces. Such LD is also contemplated and within scope of the present invention. The skilled person will appreciate that many other scenarios of functional gene-gene interaction are possible, and the particular example discussed here represents only one such possible scenario.

Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high as or possibly even higher than the at-risk variant. The at-risk variant identified may not be the functional variant itself, but is in this instance in linkage disequilibrium with the true functional variant. The functional variant may for example be a tandem repeat, such as a minisatellite or a microsatellite, a transposable element (e.g., an A/u element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected, also have predictive value for detecting association to the disease, or a susceptibility to the disease, in an individual. These tagging or surrogate markers that are in LD with the markers of the present invention can also include other markers that distinguish among haplotypes, as these similarly have predictive value for detecting susceptibility to the particular disease.

The present invention can in certain embodiments be practiced by assessing a sample comprising genomic DNA from an individual for the presence of variants described herein to be associated with thyroid cancer. Such assessment typically steps that detect the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of thyroid cancer. Detecting particular alleles of polymorphic markers can in certain embodiments be done by obtaining nucleic acid sequence data about a particular human individual, that identifies at least one allele of at least one polymorphic marker. Different alleles of the at least one marker are associated with different susceptibility to the disease in humans. Obtaining nucleic acid sequence data can comprise nucleic acid sequence at a single nucleotide position, which is sufficient to identify alleles at SNPs. The nucleic acid sequence data can also comprise sequence at any other number of nucleotide positions, in particular for genetic markers that comprise multiple nucleotide positions, and can be anywhere from two to hundreds of thousands, possibly even millions, of nucleotides (in particular, in the case of copy number variations (CNVs)).

In certain embodiments, the invention can be practiced utilizing a dataset comprising information about the genotype status of at least one polymorphic marker associated with a disease (or markers in linkage disequilibrium with at least one marker associated with the disease). In other words, a dataset containing information about such genetic status, for example in the form of sequence data, genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with the disease. A positive result for a variant (e.g., marker allele) associated with the disease, is indicative of the individual from which the dataset is derived is at increased susceptibility (increased risk) of the disease.

In certain embodiments of the invention, a polymorphic marker is correlated to a disease by referencing genotype data for the polymorphic marker to a look-up table that comprises correlations between at least one allele of the polymorphism and the disease. In some embodiments, the table comprises a correlation for one polymorhpism. In other embodiments, the table comprises a correlation for a plurality of polymorhpisms. In both scenarios, by referencing to a look-up table that gives an indication of a correlation between a marker and the disease, a risk for the disease, or a susceptibility to the disease, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

The markers described herein, e.g., the markers presented in Table 2, e.g. rs965513 (SEQ ID NO:1), may be useful for risk assessment and diagnostic purposes, either alone or in combination. Results of thyroid cancer risk based on the markers described herein can also be combined with data for other genetic markers or risk factors for thyroid cancer, to establish overall risk. Thus, even in cases where the increase in risk by individual markers is relatively modest, e.g. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease.

Thus, in certain embodiments of the invention, a plurality of variants (genetic markers, biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to thyroid cancer. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses or other methods known to the skilled person, may subsequently be used to determine the overall risk conferred based on the genotype status at the multiple loci. Assessment of risk based on such analysis may subsequently be used in the methods, uses and kits of the invention, as described herein.

Individuals who are homozygous for at-risk variants for thyroid cancer are at particularly high risk of developing thyroid cancer. This is due to the dose-dependent effect of at-risk alleles, such that the risk for homozygous carriers is generally estimated as the risk for each allelic copy squared. In one such embodiment, individuals homozygous for allele A of marker rs965513 are at particularly high risk of developing thyroid cancer compared with the general population and/or non-carriers of the rs965513-A risk allele.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined, as discussed in the above. Such markers and/or haplotypes may in those cases be also used as surrogate markers and/or haplotypes for the markers and/or haplotypes physically residing within the haplotype block as defined. As a consequence, markers and haplotypes in LD (typically characterized by inter-marker r² values of greater than 0.1, such as r² greater than 0.2, including r² greater than 0.3, also including markers correlated by values for r² greater than 0.4) with the markers and haplotypes of the present invention are also within the scope of the invention, even if they are physically located beyond the boundaries of the haplotype block as defined. This includes markers that are described herein (e.g., rs965513), but may also include other markers that are in strong LD (e.g., characterized by r² greater than 0.1 or 0.2 and/or |D′|>0.8) with rs965513 (e.g., the markers set forth in Table 2).

For the SNP markers described herein, the opposite allele to the allele found to be in excess in patients (at-risk allele) is found in decreased frequency in thyroid cancer. These markers and haplotypes in LD and/or comprising such markers, are thus protective for thyroid cancer, i.e. they confer a decreased risk or susceptibility of individuals carrying these markers and/or haplotypes developing thyroid cancer.

Certain variants of the present invention, including certain haplotypes comprise, in some cases, a combination of various genetic markers, e.g., SNPs and microsatellites. Detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype found to be associated with thyroid cancer, (e.g., marker alleles as listed in Table 1) is one in which the marker allele or haplotype is more frequently present in an individual at risk for thyroid cancer (affected), compared to the frequency of its presence in a healthy individual (control), or in randombly selected individual from the population, wherein the presence of the marker allele or haplotype is indicative of a susceptibility to thyroid cancer. In other embodiments, at-risk markers in linkage disequilibrium with one or more markers shown herein to be associated with thyroid cancer (e.g., marker alleles as listed in Table 1) are tagging markers that are more frequently present in an individual at risk for thyroid cancer (affected), compared to the frequency of their presence in a healthy individual (control) or in a randomly selected individual from the population, wherein the presence of the tagging markers is indicative of increased susceptibility to thyroid cancer. In a further embodiment, at-risk markers alleles (i.e. conferring increased susceptibility) in linkage disequilibrium with one or more markers found to be associated with thyroid cancer, are markers comprising one or more allele that is more frequently present in an individual at risk for thyroid cancer, compared to the frequency of their presence in a healthy individual (control), wherein the presence of the markers is indicative of increased susceptibility to thyroid cancer.

### Study Population

In a general sense, the methods and kits of the invention can be utilized from samples containing nucleic acid material (DNA or RNA) from any source and from any individual, or from genotype data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers and/or haplotypes in individuals who are members of a target population. Such a target population is in one embodiment a population or group of individuals at risk of developing thyroid cancer, based on other genetic factors, biomarkers, biophysical parameters, history of thyroid cancer or related diseases, previous diagnosis of thyroid cancer, family history of thyroid cancer. A target population is in certain embodiments is a population or group with known radiation exposure, such as radiation exposure due to diagnostic or therapeutic medicine, radioactive fallout from nuclear explosions, radioactive exposure due to nuclear power plants or other sources of radioactivity, etc.

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of thyroid cancer in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Styrkarsdottir, U., et al. N Engl J Med Apr. 29, 2008 (Epub ahead of print); Thorgeirsson, T., et al. Nature 452:638-42 (2008); Gudmundsson, 3., et al. Nat Genet. 40:281-3 (2008); Stacey, S, N., et al., Nat Genet. 39:865-69 (2007); Helgadottir, A., et al., Science 316:1491-93 (2007); Steinthorsdottir, V., et al., Nat Genet. 39:770-75 (2007); Gudmundsson, J., et al., Nat Genet. 39:631-37 (2007); Frayling, T M, Nature Reviews Genet 8:657-662 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including populations from Africa and Asia.

It is thus believed that the markers of the present invention found to be associated with thyroid cancer will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portugues, Italian, Polish, Bulgarian, Slavic, Serbian, Bosnian, Czech, Greek and Turkish populations. The invention furthermore in other embodiments can be practiced in specific human populations that include Bantu, Mandenk, Yoruba, San, Mbuti Pygmy, Orcadian, Adygel, Russian, Sardinian, Tuscan, Mozabite, Bedouin, Druze, Palestinian, Balochi, Brahui, Makrani, Sindhi, Pathan, Burusho, Hazara, Uygur, Kalash, Han, Dai, Daur, Hezhen, Lahu, Miao, Orogen, She, Tujia, Tu, Xibo, Yi, Mongolan, Naxi, Cambodian, Japanese, Yakut, Melanesian, Papuan, Karitianan, Surui, Colmbian, Maya and Pima.

In certain embodiments, the invention relates to populations that include black African ancestry such as populations comprising persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. In another example, self-reported persons of black African ancestry may have at least one parent of black African ancestry or at least one grandparent of black African ancestry. In another embodiment, the invention relates to individuals of Caucasian origin.

The racial contribution in individual subjects may also be determined by genetic analysis. Genetic analysis of ancestry may be carried out using unlinked microsatellite markers such as those set out in Smith et al. (Am J Hum Genet 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Thyroid Stimulating Hormone

Thyroid-stimulating hormone (also known as TSH or thyrotropin) is a peptidie hormone synthesized and secreted by thyrotrope cells in the anterior pituitary gland which regulates the endocrine function of the thyroid gland. TSH stimulates the thyroid gland to secrete the hormones thyroxine (T₄) and triiodothyronine (T₃). TSH production is controlled by a Thyrotropin Releasing Hormone, (TRH), which is manufactured in the hypothalamus and transported to the anterior pituitary gland via the superior hypophyseal artery, where it increases TSH production and release. Somatostatin is also produced by the hypothalamus, and has an opposite effect on the pituitary production of TSH, decreasing or inhibiting its release.

The level of thyroid hormones (T₃ and T₄) in the blood have an effect on the pituitary release of TSH; when the levels of T₃ and T₄ are low, the production of TSH is increased, and conversely, when levels of T₃ and T₄ are high, then TSH production is decreased. This effect creates a regulatory negative feedback loop.

Thyroxine, or 3,5,3′,5′-tetraiodothyronine (often abbreviated as T₄), is the major hormone secreted by the follicular cells of the thyroid gland. T₄ is transported in blood, with 99.95% of the secreted T₄ being protein bound, principally to thyroxine-binding globulin (TBG), and, to a lesser extent, to transthyretin and serum albumin. T₄ is involved in controlling the rate of metabolic processes in the body and influencing physical development. Administration of thyroxine has been shown to significantly increase the concentration of nerve growth factor in the brains of adult mice.

In the hypothalamus, T₄ is converted to Triiodothyronine, also known as T₃. TSH is inhibited mainly by T₃. The thyroid gland releases greater amounts of T₄ than T₃, so plasma concentrations of T₄ are 40-fold higher than those of T₃. Most of the circulating T₃ is formed peripherally by deiodination of T₄ (85%), a process that involves the removal of iodine from carbon 5 on the outer ring of T₄. Thus, T₄ acts as prohormone for T₃.

### Utility of Genetic Testing

The person skilled in the art will appreciate and understand that the variants described herein in general do not, by themselves, provide an absolute identification of individuals who will develop thyroid cancer. The variants described herein do however indicate increased and/or decreased likelihood that individuals carrying the at-risk or protective variants of the invention will develop thyroid cancer. The present inventors have discovered that certain variants confer increase risk of developing thyroid cancer, as supported by the statistically significant results presented in the Exemplification herein. This information is extremely valuable in itself, as outlined in more detail in the below, as it can be used to, for example, initiate preventive measures at an early stage, perform regular physical exams to monitor the progress and/or appearance of symptoms, or to schedule exams at a regular interval to identify early symptoms, so as to be able to apply treatment at an early stage.

The knowledge about a genetic variant that confers a risk of developing thyroid cancer offers the opportunity to apply a genetic test to distinguish between individuals with increased risk of developing thyroid cancer (i.e. carriers of the at-risk variant) and those with decreased risk of developing thyroid cancer (i.e. carriers of the protective variant). The core values of genetic testing, for individuals belonging to both of the above mentioned groups, are the possibilities of being able to diagnose a disease, or a predisposition to a disease, at an early stage and provide information to the clinician about prognosis/aggressiveness of disease in order to be able to apply the most appropriate treatment.

Individuals with a family history of thyroid cancer and carriers of at-risk variants may benefit from genetic testing since the knowledge of the presence of a genetic risk factor, or evidence for increased risk of being a carrier of one or more risk factors, may provide increased incentive for implementing a healthier lifestyle, by avoiding or minimizing known environmental risk factors for the disease. Genetic testing of patients diagnosed with thyroid cancer may furthermore give valuable information about the primary cause of the disease and can aid the clinician in selecting the best treatment options and medication for each individual.

As discussed in the above, the primary known risk factor for thyroid cancer is radiation exposure. Thyroid cancer incidence within the US has been rising for several decades (Davies, L. and Welch, H. G., Jama, 295, 2164 (2006)), which may be attributable to increased detection of sub-clinical cancers, as opposed to an increase in the true occurrence of thyroid cancer (Davies, L. and Welch, H. G., Jama, 295, 2164 (2006)). The introduction of ultrasonography and fine-needle aspiration biopsy in the 1980s improved the detection of small nodules and made cytological assessment of a nodule more routine (Rojeski, M. T. and Gharib, H., N Engl J Med, 313, 428 (1985), Ross, D. S., J Clin Endocrinol Metab, 91, 4253 (2006)). This increased diagnostic scrutiny may allow early detection of potentially lethal thyroid cancers. However, several studies report thyroid cancers as a common autopsy finding (up to 35%) in persons without a diagnosis of thyroid cancer (Bondeson, L. and Ljungberg, O., Cancer, 47, 319 (1981), Harach, H. R., et al., Cancer, 56, 531 (1985), Solares, C. A., et al., Am J Otolaryngol, 26, 87 (2005) and Sobrinho-Simoes, M. A., Sambade, M. C., and Goncalves, V., Cancer, 43, 1702 (1979)). This suggests that many people live with sub-clinical forms of thyroid cancer which are of little or no threat to their health.

Physicians use several tests to confirm the suspicion of thyroid cancer, to identify the size and location of the lump and to determine whether the lump is non-cancerous (benign) or cancerous (malignant). Blood tests such as the thyroid stimulating hormone (TSH) test check thyroid function.

TSH levels are tested in the blood of patients suspected of suffering from excess (hyperthyroidism), or deficiency (hypothyroidism) of thyroid hormone. Generally, a normal range for TSH for adults is between 0.2 and 10 uIU/mL (equivalent to mIU/L). The optimal TSH level for patients on treatment ranges between 0.3 to 3.0 mIU/L. The interpretation of TSH measurements depends also on what the blood levels of thyroid hormones (T₃ and T₄) are. The National Health Service in the UK considers a “normal” range to be more like 0.1 to 5.0 uIU/mL.

TSH levels for children normally start out much higher. In 2002, the National Academy of Clinical Biochemistry (NACB) in the United States recommended age-related reference limits starting from about 1.3-19 uIU/mL for normal term infants at birth, dropping to 0.6-10 uIU/mL at 10 weeks old, 0.4-7.0 uIU/mL at 14 months and gradually dropping during childhood and puberty to adult levels, 0.4-4.0 uIU/mL. The NACB also stated that it expected the normal (95%) range for adults to be reduced to 0.4-2.5 uIU/mL, because research had shown that adults with an initially measured TSH level of over 2.0 uIU/mL had an increased odds ratio of developing hypothyroidism over the [following] 20 years, especially if thyroid antibodies were elevated.

In general, both TSH and T₃ and T₄ should be measured to ascertain where a specific thyroid dysfunction is caused by primary pituitary or by a primary thyroid disease. If both are up (or down) then the problem is probably in the pituitary. If the one component (TSH) is up, and the other (T₃ and T₄) is down, then the disease is probably in the thyroid itself. The same holds for a low TSH, high T3 and T4 finding.

The knowledge of underlying genetic risk factors for thyroid cancer can be utilized in the application of screening programs for thyroid cancer. Thus, carriers of at-risk variants for thyroid cancer may benefit from more frequent screening than do non-carriers. Homozygous carriers of at-risk variants are particularly at risk for developing thyroid cancer.

It may be beneficial to determine TSH, T3 and T4 levels in the context of a particular genetic profile, e.g. the presence of particular at-risk alleles for thyroid cancer as described herein (e.g., rs965513-A). Since TSH, T3 and T4 are measures of thyroid function, a diagnostic and preventive screening program will benefit from analysis that includes such clinical measurements. For example, an abnormal (increased or decreased) level of TSH together with determination of the presence of at least one copy of rs965513-A is indicative of an individual is at risk of developing thyroid cancer. In one embodiment, determination of a decreased level of TSH in an individual in the context of the presence of rs965513-A is indicative of an increased risk of thyroid cancer for the individual.

Also, carriers may benefit from more extensive screening, including ultrasonography and/or fine needle biopsy. The goal of screening programs is to detect cancer at an early stage. Knowledge of genetic status of individuals with respect to known risk variants can aid in the selection of applicable screening programs. In certain embodiments, it may be useful to use the at-risk variants for thyroid cancer described herein together with one or more diagnostic tool selected from Radioactive Iodine (RAI) Scan, Ultrasound examination, CT scan (CAT scan), Magnetic Resonance Imaging (MRI), Positron Emission Tomography (PET) scan, Fine needle aspiration biopsy and surgical biopsy.

### Methods

Methods for disease risk assessment and risk management are described herein and are encompassed by the invention. The invention also encompasses methods of assessing an individual for probability of response to a therapeutic agents, methods for predicting the effectiveness of a therapeutic agents, nucleic acids, polypeptides and antibodies and computer-implemented functions. Kits for use in the various methods presented herein are also encompassed by the invention.

### Diagnostic and Screening Methods

In certain embodiments, the present invention pertains to methods of diagnosing, or aiding in the diagnosis of, thyroid cancer or a susceptibility to thyroid cancer, by detecting particular alleles at genetic markers that appear more frequently in subjects diagnosed with thyroid cancer or subjects who are susceptible to thyroid cancer. In particular embodiments, the invention is a method of determining a susceptibility to thyroid cancer by detecting at least one allele of at least one polymorphic marker (e.g., the markers described herein). In other embodiments, the invention relates to a method of diagnosing a susceptibility to thyroid cancer by detecting at least one allele of at least one polymorphic marker. The present invention describes methods whereby detection of particular alleles of particular markers or haplotypes is indicative of a susceptibility to thyroid cancer. Such prognostic or predictive assays can also be used to determine prophylactic treatment of a subject prior to the onset of symptoms of thyroid cancer.

The present invention pertains in some embodiments to methods of clinical applications of diagnosis, e.g., diagnosis performed by a medical professional. In other embodiments, the invention pertains to methods of diagnosis or determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications. The diagnostic application of disease-associated alleles as described herein, can thus for example be performed by the individual, through analysis of his/her genotype data, by a health professional based on results of a clinical test, or by a third party, including the genotype service provider. The third party may also be service provider who interprets genotype information from the customer to provide service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human conditions, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for a particular disease or trait (such as thyroid cancer). The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

In addition, in certain other embodiments, the present invention pertains to methods of determining a decreased susceptibility to thyroid cancer, by detecting particular genetic marker alleles or haplotypes that appear less frequently in patients with thyroid cancer than in individuals not diagnosed with thyroid cancer, or in the general population.

As described and exemplified herein, particular marker alleles or haplotypes (e.g. rs965513, and markers in linkage disequilibrium therewith) are associated with thyroid cancer. In one embodiment, the marker allele or haplotype is one that confers a significant risk or susceptibility to thyroid cancer. In another embodiment, the invention relates to a method of determining a susceptibility to thyroid cancer in a human individual, the method comprising determining the presence or absence of at least one allele of at least one polymorphic marker in a nucleic acid sample obtained from the individual, wherein the at least one polymorphic marker is selected from the group consisting of the polymorphic markers listed in Table 2. In another embodiment, the invention pertains to methods of determining a susceptibility to thyroid cancer in a human individual, by screening for at least one marker selected from rs965513 (SEQ ID NO: 1), rs907580 (SEQ ID NO:81) and rs7024345 (SEQ ID NO:66). In another embodiment, the marker allele or haplotype is more frequently present in a subject having, or who is susceptible to, thyroid cancer (affected), as compared to the frequency of its presence in a healthy subject (control, such as population controls). In certain embodiments, the significance of association of the at least one marker allele or haplotype is characterized by a p value<0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to thyroid cancer. These diagnostic methods involve determining whether particular alleles or haplotypes that are associated with risk of thyroid cancer are present in particular individuals. The haplotypes described herein include combinations of alleles at various genetic markers (e.g., SNPs, microsatellites or other genetic variants). The detection of the particular genetic marker alleles that make up particular haplotypes can be performed by a variety of methods described herein and/or known in the art. For example, genetic markers can be detected at the nucleic acid level (e.g., by direct nucleotide sequencing, or by other genotyping means known to the skilled in the art) or at the amino acid level if the genetic marker affects the coding sequence of a protein (e.g., by protein sequencing or by immunoassays using antibodies that recognize such a protein). The marker alleles or haplotypes of the present invention correspond to fragments of a genomic segments (e.g., genes) associated with thyroid cancer. Such fragments encompass the DNA sequence of the polymorphic marker or haplotype in question, but may also include DNA segments in strong LD (linkage disequilibrium) with the marker or haplotype. In one embodiment, such segments comprises segments in LD with the marker or haplotype as determined by a value of r² greater than 0.2 and/or |D′|>0.8).

In one embodiment, determination of a susceptibility to thyroid cancer can be accomplished using hybridization methods. (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies and methods for genotyping particular polymorphic markers.

To determine a susceptibility to thyroid cancer, a hybridization sample can be formed by contacting the test sample containing an thyroid cancer-associated nucleic acid, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of LD Block C09, as described herein, optionally comprising at least one allele of a marker described herein, or at least one haplotype described herein, or the probe can be the complementary sequence of such a sequence. The nucleic acid probe can also comprise all or a portion of the nucleotide sequence of any one of SEQ ID NO:1-229, as set forth herein. In a particular embodiment, the nucleic acid probe is a portion of the nucleotide sequence of any one of SEQ ID NO:1-229, as described herein, optionally comprising at least one allele of at least one of the polymorphic markers set forth in Table 2 herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to thyroid cancer.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

Alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with thyroid cancer. Hybridization of the PNA probe is thus diagnostic for thyroid cancer or a susceptibility to thyroid cancer.

In one embodiment of the invention, a test sample containing, genomic DNA obtained from the subject is collected and the polymerase chain reaction (PCR) is used to amplify a fragment comprising one or more markers or haplotypes of the present invention. As described herein, identification of a particular marker allele or haplotype can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis, for example by using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s). Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes. Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid that contains a polymorphic marker or haplotype, and the presence of specific alleles can then be detected directly by sequencing the polymorphic site (or multiple polymorphic sites in a haplotype) of the genomic DNA in the sample.

In another embodiment, arrays of oligonucleotide probes that are complementary to target nucleic acid sequence segments from a subject, can be used to identify particular alleles at polymorphic sites. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet. 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of thyroid cancer or a determination of a susceptibility to thyroid cancer can be made by examining expression and/or composition of a polypeptide encoded by a nucleic acid associated with thyroid cancer in those instances where the genetic marker(s) or haplotype(s) of the present invention result in a change in the composition or expression of the polypeptide. Thus, determination of a susceptibility to thyroid cancer can be made by examining expression and/or composition of one of these polypeptides, or another polypeptide encoded by a nucleic acid associated with thyroid cancer, in those instances where the genetic marker or haplotype of the present invention results in a change in the composition or expression of the polypeptide. The markers of the present invention that show association to thyroid cancer may play a role through their effect on one or more of these nearby genes. In certain embodiments, the markers show an effect on the FoxE1 gene. Possible mechanisms affecting these genes (e.g., the FoxE1 gene) include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

Thus, in another embodiment, the variants (markers or haplotypes) presented herein affect the expression of the FoxE1 gene. It is well known that regulatory element affecting gene expression may be located far away, even as far as tenths or hundreds of kilobases away, from the promoter region of a gene. By assaying for the presence or absence of at least one allele of at least one polymorphic marker of the present invention, it is thus possible to assess the expression level of such nearby genes. It is thus contemplated that the detection of the markers as described herein, or haplotypes comprising such markers, can be used for assessing and/or predicting the expression of the FoxE1 gene, or another nearby gene associated with any one of the markers shown herein to confer risk of thyroid cancer.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a particular nucleic acid. An alteration in expression of a polypeptide encoded by the nucleic acid can be, for example, an alteration in the quantitative polypeptide expression (i.e., the amount of polypeptide produced). An alteration in the composition of a polypeptide encoded by the nucleic acid is an alteration in the qualitative polypeptide expression (e.g., expression of a mutant polypeptide or of a different splicing variant). In one embodiment, diagnosis of a susceptibility to thyroid cancer is made by detecting a particular splicing variant encoded by a nucleic acid associated with thyroid cancer, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who is not affected by, and/or who does not have a susceptibility to, thyroid cancer. In one embodiment, the control sample is from a subject that does not possess a marker allele or haplotype associated with thyroid cancer, as described herein. Similarly, the presence of one or more different splicing variants in the test sample, or the presence of significantly different amounts of different splicing variants in the test sample, as compared with the control sample, can be indicative of a susceptibility to thyroid cancer. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with thyroid cancer can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of a polypeptide in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, determination of a susceptibility to thyroid cancer is made by detecting at least one marker or haplotype of the present invention, in combination with an additional protein-based, RNA-based or DNA-based assay.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with thyroid cancer, means for analyzing the nucleic acid sequence of a nucleic acid associated with thyroid cancer, means for analyzing the amino acid sequence of a polypeptide encoded by a nucleic acid associated with thyroid cancer, etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other diagnostic assays for thyroid cancer.

In one embodiment, the invention pertains to a kit for assaying a sample from a subject to detect a susceptibility to thyroid cancer in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with thyroid cancer risk. In one such embodiment, the polymorphism is selected from the group consisting of the polymorphisms as set forth in Table 2 herein. In another embodiment, the polymorphism is selected from rs965513 (SEQ ID NO:1), rs907580 (SEQ ID NO:81) and rs7024345 (SEQ ID NO:66). In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are associated with risk of thyroid cancer. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers set forth in Table 2. In another embodiment, the marker or haplotype to be detected comprises one or more markers, two or more markers, three or more markers, four or more markers or five or more markers selected from the group consisting of the markers rs965513 (SEQ ID NO:1), rs907580 (SEQ ID NO:81) and rs7024345 (SEQ ID NO:66). In another embodiment, the marker to be detected is selected from marker rs965513 (SEQ ID NO:1), or markers in linkage disequilibrium therewith.

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one such embodiment, determination of the presence of the marker or haplotype is indicative of a susceptibility (increased susceptibility or decreased susceptibility) to thyroid cancer. In another embodiment, determination of the presence of the marker or haplotype is indicative of response to a therapeutic agent for thyroid cancer. In another embodiment, the presence of the marker or haplotype is indicative of prognosis of thyroid cancer. In yet another embodiment, the presence of the marker or haplotype is indicative of progress of thyroid cancer treatment. Such treatment may include intervention by surgery, medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit.

### Therapeutic Agents

Treatment options for thyroid cancer include current standard treatment methods and those that are in clinical trials.

Current treatment options for thyroid cancer include:

Surgery—including lobectomy, where the lobe in which thyroid cancer is found is removed, thyroidectomy, where all but a very small part of the thyroid is removed, total thyroidectomoy, where the entire thyroid is removed, and lymphadenectomoy, where lymph nodes in the neck that contain cancerous growth are removed;

Radiation therapy—including externation radiation therapy and internal radiation therapy using a radioactive compound. Radiation therapy may be given after surgery to remove any surviving cancer cells. Also, follicular and papillary thyroid cancers are sometimes treated with radioactive iodine (RAI) therapy;

Chemotherapy—including the use of oral or intravenous administration of the chemotherapy compound;

Thyroid hormone therapy—this therapy includes administration of drugs preventing generation of thyroid-stimulating hormone (TSH) in the body.

A number of clinical trials for thyroid cancer therapy and treatment are currently ongoing, including but not limited to trials for ¹⁸F-fluorodeoxyglucose (FluGlucoScan); ¹¹¹In-Pentetreotide (NeuroendoMedix); Combretastatin and Paclitaxel/Carboplatin in the treatment of anaplastic thyroid cancer, ¹³¹I with or without thyroid-stimulating hormone for post-surgical treatment, XL184-301 (Exelixis), Vandetanib (Zactima; Astra Zeneca), CS-7017 (Sankyo), Decitabine (Dacogen; 5-aza-2′-deoxycytidine), Irinotecan (Pfizer, Yakult Honsha), Bortezomib (Velcade; Millenium Pharmaceuticals); 17-AAG (17-N-Allylamino-17-demethoxygeldanamycin), Sorafenib (Nexavar, Bayer), recombinant Thyrotropin, Lenalidomide (Revlimid, Celgene), Sunitinib (Sutent), Sorafenib (Nexavar, Bayer), Axitinib (AG-013736, Pfizer), Valproic Acid (2-propylpentanoic acid), Vandetanib (Zactima, Astra Zeneca), AZD6244 (Astra Zeneca), Bevacizumab (Avastin, Genetech/Roche), MK-0646 (Merck), Pazopanib (GlaxoSmithKline), Aflibercept (Sanofi-Aventis & Regeneron Pharmaceuticals), and FR901228 (Romedepsin).

The variants (markers and/or haplotypes) disclosed herein to confer increased risk of thyroid cancer can also be used to identify novel therapeutic targets for thyroid cancer. For example, genes containing, or in linkage disequilibrium with, one or more of these variants, or their products (e.g., the FoxE1 gene and its gene product), as well as genes or their products that are directly or indirectly regulated by or interact with these variant genes or their products, can be targeted for the development of therapeutic agents to treat thyroid cancer, or prevent or delay onset of symptoms associated with thyroid cancer. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002)

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein (e.g., the markers and haplotypes set forth in Table 2) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., Curr. Opin. Genet. Dev. 10:562-7 (2000), Bosher et al., Nat. Cell Biol. 2:E31-6 (2000), and Hunter, Curr. Biol. 9:R440-442 (1999).

A genetic defect leading to increased predisposition or risk for development of a disease, such as thyroid cancer, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat thyroid cancer. Thus, the variants of the invention are useful as targets for the identification and/or development of therapeutic agents. In certain embodiments, such methods include assaying the ability of an agent or compound to modulate the activity and/or expression of a nucleic acid that includes at least one of the variants (markers and/or haplotypes) of the present invention, or the encoded product of the nucleic acid. In certain embodiments, the agent or compound modulates the activity or expression of the FoxE1 gene. The agents or compounds may also inhibit or alter the undesired activity or expression of the encoded nucleic acid product, i.e. the FoxE1 protein product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating thyroid cancer can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site or haplotype (e.g., the rs965513 polymorphic marker, or markers in linkage disequilibrium therewith) is indicative of a different response, e.g. a different response rate, to a particular treatment modality. This means that a patient diagnosed with thyroid cancer, and carrying a certain allele at a polymorphic or haplotype of the present invention (e.g., the at-risk and protective alleles and/or haplotypes of the invention) would respond better to, or worse to, a specific therapeutic, drug and/or other therapy used to treat the disease. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

Any of the treatment methods and compounds described in the above under Therapeutic agents can be used in such methods. I.e., a treatment for thyroid cancer using any of the compounds or methods described or contemplated in the above may, in certain embodiments, benefit from screening for the presence of particular alleles for at least one of the polymorphic markers described herein, wherein the presence of the particular allele is predictive of the treatment outcome for the particular compound or method.

In certain embodiments, a therapeutic agent (drug) for treating thyroid cancer is provided together with a kit for determining the allelic status at a polymorphic marker as described herein (e.g., rs965513, or markers in linkage disequilibrium therewith). If an individual is positive for the particular allele or plurality of alleles being tested, the individual is more likely to benefit from the particular compound than non-carriers of the allele. In certain other embodiments, genotype information about the at least one polymorphic marker predictive of the treatment outcome of the particular compound is predetermined and stored in a database, in a look-up table or by other suitable means, and can for example be accessed from a database or look-up table by conventional data query methods known to the skilled person. If a particular individual is determined to carry certain alleles predictive of positive treatment outcome of a particular compound or drug for treating thyroid cancer, then the individual is likely to benefit from administration of the particular compound.

The present invention also relates to methods of monitoring progress or effectiveness of a treatment for thyroid cancer. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for thyroid cancer as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention may be more likely to respond favorably to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with thyroid cancer when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of lifestyle changes and administration of particular treatment, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

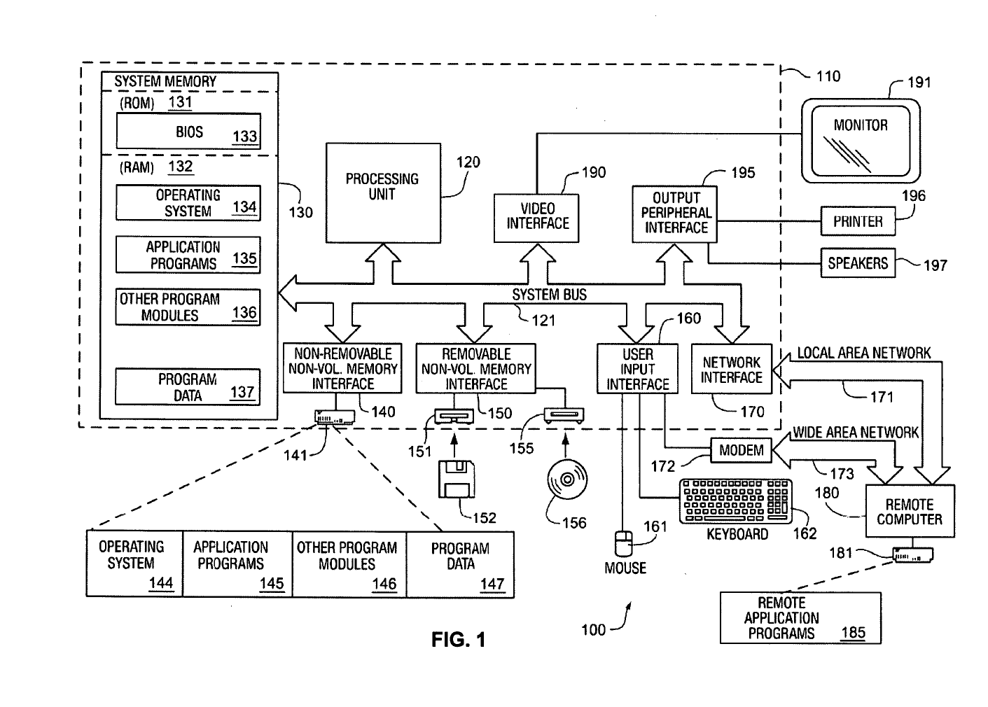

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral. Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, the invention relates to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a guardian of the individual, a health care provider or genetic analysis service provider), or for deriving information from the genotype data, e.g., by comparing the genotype data to information about genetic risk factors contributing to increased susceptibility to the thyroid cancer, and reporting results based on such comparison.

In general terms, computer-readable media has capabilities of storing (i) identifier information for at least one polymorphic marker or a haplotype, as described herein; (ii) an indicator of the frequency of at least one allele of said at least one marker, or the frequency of a haplotype, in individuals with thyroid cancer; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

The markers and haplotypes described herein to be associated with increased susceptibility (e.g., increased risk) of thyroid cancer, are in certain embodiments useful for interpretation and/or analysis of genotype data. Thus in certain embodiments, an identification of an at-risk allele for thyroid cancer, as shown herein, or an allele at a polymorphic marker in LD with any one of the markers shown herein to be associated with thyroid cancer, is indicative of the individual from whom the genotype data originates is at increased risk of thyroid cancer. In one such embodiment, genotype data is generated for at least one polymorphic marker shown herein to be associated with thyroid cancer, or a marker in linkage disequilibrium therewith. The genotype data is subsequently made available to a third party, such as the individual from whom the data originates, his/her guardian or representative, a physician or health care worker, genetic counselor, or insurance agent, for example via a user interface accessible over the internet, together with an interpretation of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk varians in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of any one of SEQ ID NO:1-229, or a nucleotide sequence comprising, or consisting of, the complement of the nucleotide sequence of any one of SEQ ID NO:1-229, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Antibodies

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein, in particular thyroid cancer. Antibodies specific for a variant protein of the present invention that is encoded by a nucleic acid that comprises at least one polymorphic marker or haplotype as described herein can be used to screen for the presence of the variant protein, for example to screen for a predisposition to thyroid cancer as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting examples.

### Example 1

### Identification of Risk Variants on Chromosome 9q22.33 that Confer Risk of Thyroid Cancer

The incidence of thyroid cancer in Iceland is higher than in the neighboring countries and among the highest in the world. Age standardized incidence in Iceland per 100,000 is 5 and 12.5 for males and females respectively. The average age at diagnosis is 61 for males and 47 for females. The distribution between histological subtypes is similar in Iceland as in other industrialized countries. The papillary histological subtype is the most frequent, representing up to 80% of all thyroid cancers, second most frequent it the follicular type (˜14%), third is the anaplastic type representing about 5% of all thyroid cases, and least common is the medullary type (−1%).

### Subjects

Approval for this study was granted by the National Bioethics Committee of Iceland and the Icelandic Data Protection Authority.

Our collection of samples used for the thyroid cancer study represents the overall distribution in Iceland quite well. Of the 406 cases that we genotyped, 309 (82%) are of papillary type, 53 (14%) are of follicular type, 7 (1.5%) are medullary thyroid cancer, and 37 are of unknown or undetermined histological subphenotype.

The results presented below in Table 1 are for the combined results for all our cases since no statistically significant difference was observed between the different histological subgroups.

The 28,858 Icelandic controls consisted individuals from other ongoing genome-wide association studies at deCODE genetics. Individuals with a diagnosis of thyroid cancer were excluded. Both male and female genders were included.

### Genotyping

In a genome-wide search for susceptibility variants for thyroid cancer, samples from Icelandic patients diagnosed with thyroid cancer and population controls were genotyped on Illumina Hap300 SNP bead microarrays (Illumina, San Diego, Calif., USA), containing 317,503 SNPs derived from Phase I of the International HapMap project. This chip provides about 75% genomic coverage in the Utah CEPH (CEU) HapMap samples for common SNPs at r²≧0.8 (Barrett and Cardon, (2006), Nat Genet, 38, 659-62). Markers that were deemed unsuitable either because they were monomorphic (minor allele frequency in the combined patient and control groups less than 0.001) or because they had low (<95%) yield were removed prior to analysis.

Markers rs907580, rs7024345 and rs965513 were further assessed by Centaurus SNP genotyping (Kutyavin, et al., (2006), Nucleic Acids Res, 34, e128).

All genotyping was carried out at the deCODE genetics facility.

### Statistical Analysis

We calculated the odds ratio (OR) of a SNP allele assuming the multiplicative model, i.e. assuming that the relative risk of the two alleles that a person carries multiplies. Allelic frequencies rather than carrier frequencies are presented for the markers. The associated P-values were calculated with a standard likelihood ratio Chi-squared statistic as implemented in the NEMO software package (Gretarsdottir, et al., (2003), Nat Genet, 35, 131-8). Confidence intervals were calculated assuming that the estimate of the OR has a log-normal distribution.

All P-values are reported as two-sided.

### Results

Upon analysis of genotype from the Illumina Hap300 chip, we found three markers, rs965513, rs907580 and rs7024345 on chromosome 9q22.33 that gave very significant association to thyroid cancer. We followed up those results by genotyping additional cases using Centaurus genotyping assays. The results are shown in Table 1A.

All three markers give genome-wide significant association to thyroid cancer (correction for 317,000 tests requires P-value of less than 0.05/317,000˜1.5×10-8), with the most significant results obtained for rs965513 (OR 1.77, P-value 1.18×10-15). The rs907580 and rs702345 markers are correlated with rs965513, with r2-values of 0.90 (Table 1B), and these markers are therefore most likely capturing the same association signal.

TABLE 1A
Association of variants on chromosome
9q22.33 with thyroid cancer.

| | | P | | # | Case | # | Ctrls |
|---|---|---|---|---|---|---|---|
| Marker | Allele | value | OR | Case | freq | Ctrls | freq |
| rs965513 | 1 | 1.18E−15 | 1.77 | 404 | 0.491 | 28858 | 0.353 |
| rs965513 | 3 | 1.18E−15 | 0.56 | 404 | 0.509 | 28858 | 0.647 |
| rs907580 | 1 | 4.56E−12 | 1.67 | 403 | 0.397 | 28833 | 0.283 |
| rs907580 | 3 | 4.56E−12 | 0.60 | 403 | 0.603 | 28833 | 0.717 |
| rs7024345 | 1 | 1.62E−09 | 1.56 | 406 | 0.385 | 28852 | 0.286 |
| rs7024345 | 3 | 1.62E−09 | 0.64 | 406 | 0.615 | 28852 | 0.714 |

Shown are markers, the associating allele, P-value for the association, Odds Ratio for the allelic risk, number of cases and controls, and allelic frequency in cases and controls.

TABLE 1B
LD characteristics for the three markers giving strongest
association to thyroid cancer. LD was determined in the
Caucasian HapMap sample (http://www.hapmap.org)

| | | | | | Position |
|---|---|---|---|---|---|
| M-1 | M-2 | D′ | r2 | P-value | (B36) |
| rs7024345 | rs907580 | 1 | 0.948461 | 1.40E−45 | 99635059 |
| rs7024345 | rs965513 | 0.90033 | 0.454289 | 7.25E−14 | 99635059 |
| rs907580 | rs965513 | 0.897329 | 0.433569 | 3.20E−13 | 99662418 |

TABLE 2
Surrogate SNPs in linkage disequilibrium (LD) with rs965513.

| | | | | | Position | |
|---|---|---|---|---|---|---|
| Marker | Anchor | D′ | r² | P-value | (bp) (B36) | SEQ ID NO: |
| rs965513 | rs965513 | 1 | 1 | — | 99595930 | 1 |
| rs7030256 | rs965513 | 1 | 1 | 1.69E−36 | 99575024 | 2 |
| rs1588635 | rs965513 | 1 | 1 | 1.36E−37 | 99577623 | 3 |
| rs7028661 | rs965513 | 1 | 1 | 1.36E−37 | 99578291 | 4 |
| rs7021576 | rs965513 | 1 | 1 | 2.40E−37 | 99580362 | 5 |
| rs1561962 | rs965513 | 1 | 1 | 1.36E−37 | 99586040 | 6 |
| rs925488 | rs965513 | 1 | 1 | 1.36E−37 | 99586212 | 7 |
| rs925489 | rs965513 | 1 | 1 | 1.36E−37 | 99586421 | 8 |
| rs7020976 | rs965513 | 1 | 1 | 1.36E−37 | 99587793 | 9 |
| rs7032019 | rs965513 | 1 | 1 | 1.36E−37 | 99587965 | 10 |
| rs7850258 | rs965513 | 1 | 1 | 3.15E−37 | 99588834 | 11 |
| rs1443438 | rs965513 | 1 | 1 | 1.36E−37 | 99589849 | 12 |
| rs7030241 | rs965513 | 1 | 1 | 3.15E−37 | 99590196 | 13 |
| rs10739496 | rs965513 | 1 | 1 | 1.36E−37 | 99592380 | 14 |
| rs10983761 | rs965513 | 1 | 1 | 1.36E−37 | 99593778 | 15 |
| rs4743131 | rs965513 | 1 | 1 | 1.36E−37 | 99594728 | 16 |
| rs10759944 | rs965513 | 1 | 1 | 1.36E−37 | 99596793 | 17 |
| rs1877431 | rs965513 | 1 | 0.903743 | 1.67E−32 | 99573968 | 18 |
| rs10124220 | rs965513 | 0.919308 | 0.741713 | 2.01E−21 | 99622895 | 19 |
| rs7848973 | rs965513 | 0.960618 | 0.682525 | 5.99E−22 | 99628660 | 20 |
| rs1443432 | rs965513 | 0.958402 | 0.614607 | 9.71E−20 | 99623016 | 21 |
| rs7357631 | rs965513 | 0.820443 | 0.608334 | 2.18E−18 | 99568141 | 22 |
| rs1912995 | rs965513 | 0.820443 | 0.608334 | 2.18E−18 | 99570720 | 23 |
| rs4297160 | rs965513 | 0.957388 | 0.593088 | 9.30E−19 | 99625327 | 24 |
| rs7045138 | rs965513 | 0.957388 | 0.593088 | 9.30E−19 | 99631284 | 25 |
| rs10983700 | rs965513 | 0.868166 | 0.560683 | 8.24E−15 | 99577276 | 26 |
| rs7860144 | rs965513 | 0.729785 | 0.485957 | 2.24E−13 | 99666705 | 27 |
| rs894673 | rs965513 | 0.743114 | 0.48261 | 5.71E−14 | 99652091 | 28 |
| rs3758251 | rs965513 | 0.743114 | 0.48261 | 5.71E−14 | 99653521 | 29 |
| rs1443434 | rs965513 | 0.743114 | 0.48261 | 5.71E−14 | 99657300 | 30 |
| rs2417575 | rs965513 | 0.716866 | 0.480309 | 4.42E−14 | 99668463 | 31 |
| rs2417576 | rs965513 | 0.716866 | 0.480309 | 4.42E−14 | 99668528 | 32 |
| rs1443436 | rs965513 | 0.716866 | 0.480309 | 4.42E−14 | 99671119 | 33 |
| rs925487 | rs965513 | 0.716866 | 0.480309 | 4.42E−14 | 99676219 | 34 |
| rs10123699 | rs965513 | 0.716866 | 0.480309 | 4.42E−14 | 99677680 | 35 |
| rs12342417 | rs965513 | 0.716866 | 0.480309 | 4.42E−14 | 99678886 | 36 |
| rs10984103 | rs965513 | 0.716866 | 0.480309 | 4.42E−14 | 99679096 | 37 |
| rs2120264 | rs965513 | 0.716866 | 0.480309 | 4.42E−14 | 99685549 | 38 |
| rs3758249 | rs965513 | 0.742362 | 0.478964 | 9.03E−14 | 99653961 | 39 |
| rs907577 | rs965513 | 0.735586 | 0.474896 | 1.83E−13 | 99654938 | 40 |
| rs1443435 | rs965513 | 0.735586 | 0.474896 | 1.83E−13 | 99657404 | 41 |
| rs12348691 | rs965513 | 0.736568 | 0.473731 | 3.29E−13 | 99648503 | 42 |
| rs13288000 | rs965513 | 0.736568 | 0.473731 | 3.29E−13 | 99648801 | 43 |
| rs1867278 | rs965513 | 0.730735 | 0.470997 | 3.41E−13 | 99655770 | 44 |
| rs7873389 | rs965513 | 0.734583 | 0.465596 | 1.55E−12 | 99649051 | 45 |
| rs10818133 | rs965513 | 0.72673 | 0.461714 | 1.80E−12 | 99650169 | 46 |
| rs907581 | rs965513 | 0.686123 | 0.455091 | 2.86E−13 | 99662010 | 47 |
| rs993501 | rs965513 | 0.686123 | 0.455091 | 2.86E−13 | 99663198 | 48 |
| rs10759975 | rs965513 | 0.686123 | 0.455091 | 2.86E−13 | 99665014 | 49 |
| rs13287360 | rs965513 | 0.686123 | 0.455091 | 2.86E−13 | 99677502 | 50 |
| rs4743139 | rs965513 | 0.686123 | 0.455091 | 2.86E−13 | 99678241 | 51 |
| rs7866436 | rs965513 | 0.686123 | 0.455091 | 2.86E−13 | 99689917 | 52 |
| rs12006522 | rs965513 | 0.686123 | 0.455091 | 2.86E−13 | 99692532 | 53 |
| rs12004762 | rs965513 | 0.686123 | 0.455091 | 2.86E−13 | 99692576 | 54 |
| rs7034648 | rs965513 | 0.686123 | 0.455091 | 2.86E−13 | 99693914 | 55 |
| rs7032086 | rs965513 | 0.686123 | 0.455091 | 2.86E−13 | 99696223 | 56 |
| rs7036589 | rs965513 | 0.686123 | 0.455091 | 2.86E−13 | 99697541 | 57 |
| rs7037324 | rs965513 | 0.686123 | 0.455091 | 2.86E−13 | 99698139 | 58 |
| rs10739526 | rs965513 | 0.686123 | 0.455091 | 2.86E−13 | 99702492 | 59 |
| rs3824495 | rs965513 | 0.686123 | 0.455091 | 2.86E−13 | 99703521 | 60 |
| rs3808893 | rs965513 | 0.686123 | 0.455091 | 2.86E−13 | 99703566 | 61 |
| rs9299258 | rs965513 | 0.686123 | 0.455091 | 2.86E−13 | 99706364 | 62 |
| rs1561961 | rs965513 | 0.686123 | 0.455091 | 2.86E−13 | 99707420 | 63 |
| rs6478423 | rs965513 | 0.90033 | 0.454289 | 7.25E−14 | 99631851 | 64 |
| rs10739513 | rs965513 | 0.90033 | 0.454289 | 7.25E−14 | 99632526 | 65 |
| rs7024345 | rs965513 | 0.90033 | 0.454289 | 7.25E−14 | 99635059 | 66 |
| rs1912996 | rs965513 | 0.90033 | 0.454289 | 7.25E−14 | 99638082 | 67 |
| rs7023267 | rs965513 | 0.90033 | 0.454289 | 7.25E−14 | 99643756 | 68 |
| rs7048394 | rs965513 | 0.90033 | 0.454289 | 7.25E−14 | 99645254 | 69 |
| rs1348386 | rs965513 | 0.90033 | 0.454289 | 7.25E−14 | 99652628 | 70 |
| rs10512255 | rs965513 | 0.680722 | 0.450668 | 5.41E−13 | 99692403 | 71 |
| rs7027221 | rs965513 | 0.680722 | 0.450668 | 5.41E−13 | 99702200 | 72 |
| rs7038998 | rs965513 | 0.689315 | 0.450038 | 2.02E−12 | 99702217 | 73 |
| rs4460498 | rs965513 | 0.72036 | 0.439276 | 3.29E−12 | 99660233 | 74 |
| rs973473 | rs965513 | 0.893814 | 0.437253 | 6.69E−13 | 99660551 | 75 |
| rs3021523 | rs965513 | 0.897329 | 0.433569 | 3.20E−13 | 99656404 | 76 |
| rs925485 | rs965513 | 0.897329 | 0.433569 | 3.20E−13 | 99659382 | 77 |
| rs1465965 | rs965513 | 0.897329 | 0.433569 | 3.20E−13 | 99660147 | 78 |
| rs1912998 | rs965513 | 0.897329 | 0.433569 | 3.20E−13 | 99661207 | 79 |
| rs907582 | rs965513 | 0.897329 | 0.433569 | 3.20E−13 | 99661747 | 80 |
| rs907580 | rs965513 | 0.897329 | 0.433569 | 3.20E−13 | 99662418 | 81 |
| rs907578 | rs965513 | 0.897329 | 0.433569 | 3.20E−13 | 99662704 | 82 |
| rs7859751 | rs965513 | 0.85327 | 0.424426 | 1.70E−12 | 99615709 | 83 |
| rs7031386 | rs965513 | 0.658695 | 0.422265 | 1.17E−11 | 99705490 | 84 |
| rs7034336 | rs965513 | 0.651224 | 0.421869 | 1.97E−11 | 99700921 | 85 |
| rs6478445 | rs965513 | 0.644902 | 0.409551 | 2.93E−10 | 99664120 | 86 |
| rs10984253 | rs965513 | 0.628715 | 0.389602 | 6.79E−09 | 99704295 | 87 |
| rs10113884 | rs965513 | 0.628716 | 0.387517 | 4.43E−11 | 99664443 | 88 |
| rs10119760 | rs965513 | 0.598931 | 0.351323 | 6.43E−09 | 99664423 | 89 |
| rs2120262 | rs965513 | 0.652035 | 0.336135 | 4.13E−10 | 99715797 | 90 |
| rs12352658 | rs965513 | 1 | 0.327731 | 4.14E−13 | 99591589 | 91 |
| rs10818094 | rs965513 | 1 | 0.302326 | 2.85E−12 | 99603649 | 92 |
| rs10818048 | rs965513 | 1 | 0.290061 | 7.28E−12 | 99578538 | 93 |
| rs12347079 | rs965513 | 1 | 0.290061 | 1.01E−11 | 99590048 | 94 |
| rs16924274 | rs965513 | 1 | 0.290061 | 7.28E−12 | 99597112 | 95 |
| rs1877432 | rs965513 | 1 | 0.278075 | 1.83E−11 | 99583701 | 96 |
| rs7023279 | rs965513 | 0.781059 | 0.270085 | 6.89E−09 | 99562491 | 97 |
| rs10760017 | rs965513 | 0.639369 | 0.267287 | 3.53E−08 | 99727744 | 98 |
| rs10983826 | rs965513 | 1 | 0.261538 | 4.60E−10 | 99608601 | 99 |
| rs2805789 | rs965513 | 0.539381 | 0.261518 | 9.26E−07 | 99539118 | 100 |
| rs10818041 | rs965513 | 0.926072 | 0.259277 | 6.80E−09 | 99565474 | 101 |
| rs10818042 | rs965513 | 0.926072 | 0.259277 | 6.80E−09 | 99565489 | 102 |
| rs4319207 | rs965513 | 0.926072 | 0.259277 | 6.80E−09 | 99569522 | 103 |
| rs1398230 | rs965513 | 0.817958 | 0.255815 | 1.36E−08 | 99559834 | 104 |
| rs7847449 | rs965513 | 1 | 0.254902 | 1.10E−10 | 99591729 | 105 |
| rs2668797 | rs965513 | 0.549104 | 0.254841 | 9.57E−08 | 99522324 | 106 |
| rs2808681 | rs965513 | 0.549104 | 0.254841 | 9.57E−08 | 99522382 | 107 |
| rs953198 | rs965513 | 0.549104 | 0.254841 | 9.57E−08 | 99522490 | 108 |
| rs2668795 | rs965513 | 0.549104 | 0.254841 | 9.57E−08 | 99523737 | 109 |
| rs2668794 | rs965513 | 0.549104 | 0.254841 | 9.57E−08 | 99524445 | 110 |
| rs2808682 | rs965513 | 0.549104 | 0.254841 | 9.57E−08 | 99525283 | 111 |
| rs2808693 | rs965513 | 0.549104 | 0.254841 | 9.57E−08 | 99530579 | 112 |
| rs2808697 | rs965513 | 0.549104 | 0.254841 | 9.57E−08 | 99532364 | 113 |
| rs2805815 | rs965513 | 0.549104 | 0.254841 | 9.57E−08 | 99535981 | 114 |
| rs2805812 | rs965513 | 0.549104 | 0.254841 | 9.57E−08 | 99536224 | 115 |
| rs2805811 | rs965513 | 0.549104 | 0.254841 | 9.57E−08 | 99536295 | 116 |
| rs2805809 | rs965513 | 0.549104 | 0.254841 | 9.57E−08 | 99536743 | 117 |
| rs2668804 | rs965513 | 0.549104 | 0.254841 | 9.57E−08 | 99537571 | 118 |
| rs2805798 | rs965513 | 0.549104 | 0.254841 | 9.57E−08 | 99538242 | 119 |
| rs2805797 | rs965513 | 0.549104 | 0.254841 | 9.57E−08 | 99538313 | 120 |
| rs2668803 | rs965513 | 0.549104 | 0.254841 | 9.57E−08 | 99538541 | 121 |
| rs2805796 | rs965513 | 0.549104 | 0.254841 | 9.57E−08 | 99538564 | 122 |
| rs2668802 | rs965513 | 0.549104 | 0.254841 | 9.57E−08 | 99538652 | 123 |
| rs2805790 | rs965513 | 0.549104 | 0.254841 | 9.57E−08 | 99539027 | 124 |
| rs2805784 | rs965513 | 0.549104 | 0.254841 | 9.57E−08 | 99539388 | 125 |
| rs2805781 | rs965513 | 0.549104 | 0.254841 | 9.57E−08 | 99540099 | 126 |
| rs2805778 | rs965513 | 0.549104 | 0.254841 | 9.57E−08 | 99540956 | 127 |
| rs2805771 | rs965513 | 0.549104 | 0.254841 | 9.57E−08 | 99543836 | 128 |
| rs2805768 | rs965513 | 0.549104 | 0.254841 | 9.57E−08 | 99545013 | 129 |
| rs2808700 | rs965513 | 0.549104 | 0.254841 | 9.57E−08 | 99545841 | 130 |
| rs2808698 | rs965513 | 0.571747 | 0.251516 | 4.22E−07 | 99533271 | 131 |
| rs2805782 | rs965513 | 0.545323 | 0.251343 | 1.76E−07 | 99539791 | 132 |
| rs2805822 | rs965513 | 0.521897 | 0.236156 | 7.65E−07 | 99531161 | 133 |
| rs6478391 | rs965513 | 0.859196 | 0.232449 | 9.73E−08 | 99571837 | 134 |
| rs874004 | rs965513 | 0.723916 | 0.232011 | 4.26E−07 | 99661939 | 135 |
| rs7357707 | rs965513 | 0.723916 | 0.232011 | 4.26E−07 | 99670770 | 136 |
| rs7033315 | rs965513 | 0.723916 | 0.232011 | 4.26E−07 | 99676061 | 137 |
| rs10119795 | rs965513 | 0.723916 | 0.232011 | 4.26E−07 | 99677160 | 138 |
| rs2805773 | rs965513 | 0.521658 | 0.23178 | 8.31E−07 | 99543639 | 139 |
| rs10818021 | rs965513 | 0.763123 | 0.231121 | 9.71E−08 | 99552241 | 140 |
| rs1512261 | rs965513 | 0.529317 | 0.227753 | 8.46E−07 | 99562351 | 141 |
| rs2805799 | rs965513 | 0.508782 | 0.226252 | 7.80E−07 | 99537549 | 142 |
| rs2668799 | rs965513 | 0.51014 | 0.219958 | 8.86E−07 | 99530562 | 143 |
| rs7871887 | rs965513 | 0.831739 | 0.203084 | 7.74E−07 | 99611263 | 144 |
| rs2808695 | rs965513 | 0.496163 | 0.201236 | 3.94E−06 | 99531899 | 145 |
| rs7853349 | rs965513 | 0.700383 | 0.19468 | 5.02E−06 | 99690080 | 146 |
| rs6586 | rs965513 | 0.700383 | 0.19468 | 5.02E−06 | 99706752 | 147 |
| rs1561958 | rs965513 | 0.700383 | 0.19468 | 5.02E−06 | 99709620 | 148 |
| rs12238579 | rs965513 | 0.68351 | 0.187585 | 0.000022 | 99691879 | 149 |
| rs12235588 | rs965513 | 0.775693 | 0.181909 | 3.66E−06 | 99556717 | 150 |
| rs1572025 | rs965513 | 0.464188 | 0.181413 | 4.47E−06 | 99780936 | 151 |
| rs7855088 | rs965513 | 0.464188 | 0.181413 | 4.47E−06 | 99782074 | 152 |
| rs879275 | rs965513 | 0.464188 | 0.181413 | 4.47E−06 | 99821541 | 153 |
| rs1561960 | rs965513 | 0.758648 | 0.178461 | 4.82E−06 | 99608297 | 154 |
| rs10739476 | rs965513 | 0.762206 | 0.175996 | 0.000015 | 99506712 | 155 |
| rs17335265 | rs965513 | 0.475362 | 0.164756 | 0.000056 | 99430448 | 156 |
| rs10817781 | rs965513 | 0.556801 | 0.163618 | 0.000014 | 99369961 | 157 |
| rs1800975 | rs965513 | 0.528842 | 0.151107 | 0.000066 | 99499399 | 158 |
| rs2805779 | rs965513 | 0.521666 | 0.148646 | 0.000049 | 99406774 | 159 |
| rs2805767 | rs965513 | 0.499638 | 0.148567 | 0.00016 | 99456420 | 160 |
| rs952765 | rs965513 | 0.447965 | 0.146545 | 0.000131 | 99407127 | 161 |
| rs16923677 | rs965513 | 0.464509 | 0.146437 | 0.000135 | 99507465 | 162 |
| rs958346 | rs965513 | 0.498946 | 0.145578 | 0.000083 | 99401685 | 163 |
| rs2805810 | rs965513 | 0.572926 | 0.143763 | 0.000075 | 99371559 | 164 |
| rs774122 | rs965513 | 0.516668 | 0.14374 | 0.000092 | 99951551 | 165 |
| rs2808692 | rs965513 | 0.514491 | 0.138686 | 0.000103 | 99530193 | 166 |
| rs6478262 | rs965513 | 0.587984 | 0.138603 | 0.00018 | 99359101 | 167 |
| rs3176633 | rs965513 | 0.792122 | 0.137393 | 0.000093 | 99499130 | 168 |
| rs10817858 | rs965513 | 0.643771 | 0.135825 | 0.000175 | 99427940 | 169 |
| rs3176757 | rs965513 | 0.643771 | 0.135825 | 0.000175 | 99476879 | 170 |
| rs10759868 | rs965513 | 0.643771 | 0.135825 | 0.000175 | 99503899 | 171 |
| rs2668792 | rs965513 | 0.506147 | 0.135203 | 0.000085 | 99525832 | 172 |
| rs2808686 | rs965513 | 0.506147 | 0.135203 | 0.000085 | 99527112 | 173 |
| rs2805824 | rs965513 | 0.506147 | 0.135203 | 0.000085 | 99527211 | 174 |
| rs2808687 | rs965513 | 0.506147 | 0.135203 | 0.000085 | 99527771 | 175 |
| rs2808691 | rs965513 | 0.506147 | 0.135203 | 0.000085 | 99530127 | 176 |
| rs2805840 | rs965513 | 0.506147 | 0.135203 | 0.000085 | 99545367 | 177 |
| rs2808701 | rs965513 | 0.506147 | 0.135203 | 0.000085 | 99546029 | 178 |
| rs7856619 | rs965513 | 0.668361 | 0.133227 | 0.000179 | 99542122 | 179 |
| rs10983030 | rs965513 | 0.483519 | 0.132142 | 0.000142 | 99416368 | 180 |
| rs2773347 | rs965513 | 0.483519 | 0.132142 | 0.000142 | 99428018 | 181 |
| rs2773351 | rs965513 | 0.483519 | 0.132142 | 0.000142 | 99439955 | 182 |
| rs2026132 | rs965513 | 0.483519 | 0.132142 | 0.000142 | 99455661 | 183 |
| rs2805839 | rs965513 | 0.483519 | 0.132142 | 0.000142 | 99461848 | 184 |
| rs2805837 | rs965513 | 0.483519 | 0.132142 | 0.000142 | 99473054 | 185 |
| rs2808668 | rs965513 | 0.483519 | 0.132142 | 0.000142 | 99492256 | 186 |
| rs2808673 | rs965513 | 0.483519 | 0.132142 | 0.000142 | 99508039 | 187 |
| rs2808675 | rs965513 | 0.483519 | 0.132142 | 0.000142 | 99510843 | 188 |
| rs2805828 | rs965513 | 0.483519 | 0.132142 | 0.000142 | 99511248 | 189 |
| rs2808677 | rs965513 | 0.483519 | 0.132142 | 0.000142 | 99513232 | 190 |
| rs2808678 | rs965513 | 0.483519 | 0.132142 | 0.000142 | 99515841 | 191 |
| rs7031623 | rs965513 | 0.432801 | 0.131898 | 0.000319 | 99452196 | 192 |
| rs10116536 | rs965513 | 0.432801 | 0.131898 | 0.000319 | 99468798 | 193 |
| rs10120102 | rs965513 | 0.432801 | 0.131898 | 0.000319 | 99469521 | 194 |
| rs16923269 | rs965513 | 0.432801 | 0.131898 | 0.000319 | 99471953 | 195 |
| rs3176748 | rs965513 | 0.432801 | 0.131898 | 0.000319 | 99478165 | 196 |
| rs3176639 | rs965513 | 0.432801 | 0.131898 | 0.000319 | 99497930 | 197 |
| rs4480232 | rs965513 | 0.432801 | 0.131898 | 0.000319 | 99511653 | 198 |
| rs12350946 | rs965513 | 0.432801 | 0.131898 | 0.000319 | 99514710 | 199 |
| rs10983424 | rs965513 | 0.432801 | 0.131898 | 0.000319 | 99514885 | 200 |
| rs12346336 | rs965513 | 0.432801 | 0.131898 | 0.000319 | 99519604 | 201 |
| rs7849509 | rs965513 | 0.432801 | 0.131898 | 0.000319 | 99519946 | 202 |
| rs16923815 | rs965513 | 0.432801 | 0.131898 | 0.000319 | 99520178 | 203 |
| rs2808689 | rs965513 | 0.501114 | 0.130527 | 0.00012 | 99528253 | 204 |
| rs7871185 | rs965513 | 0.552968 | 0.127427 | 0.000268 | 99359715 | 205 |
| rs4743119 | rs965513 | 0.733465 | 0.125205 | 0.000157 | 99415073 | 206 |
| rs4284139 | rs965513 | 0.419164 | 0.122545 | 0.000639 | 99510156 | 207 |
| rs2805777 | rs965513 | 0.471849 | 0.121611 | 0.000276 | 99420855 | 208 |
| rs12349178 | rs965513 | 0.417613 | 0.118616 | 0.00083 | 99516173 | 209 |
| rs10982745 | rs965513 | 0.520093 | 0.117909 | 0.000203 | 99363907 | 210 |
| rs10217225 | rs965513 | 1 | 0.117647 | 7.58E−06 | 99636812 | 211 |
| rs12344605 | rs965513 | 0.411211 | 0.115869 | 0.001023 | 99421419 | 212 |
| rs10119687 | rs965513 | 0.411211 | 0.115869 | 0.001023 | 99512650 | 213 |
| rs2795492 | rs965513 | 0.338283 | 0.114436 | 0.000489 | 99953197 | 214 |
| rs2282192 | rs965513 | 0.626552 | 0.113868 | 0.000389 | 99712159 | 215 |
| rs2120263 | rs965513 | 0.626552 | 0.113868 | 0.000389 | 99715765 | 216 |
| rs7034310 | rs965513 | 0.635696 | 0.11308 | 0.000963 | 99566978 | 217 |
| rs1536950 | rs965513 | 0.484818 | 0.111772 | 0.000389 | 99373593 | 218 |
| rs12349452 | rs965513 | 0.530427 | 0.111661 | 0.000279 | 99850226 | 219 |
| rs10818071 | rs965513 | 1 | 0.111111 | 0.000016 | 99590074 | 220 |
| rs7855669 | rs965513 | 0.491557 | 0.110059 | 0.001647 | 99350532 | 221 |
| rs2036959 | rs965513 | 0.455187 | 0.109348 | 0.000439 | 99525228 | 222 |
| rs7035650 | rs965513 | 0.354957 | 0.106491 | 0.000572 | 99806663 | 223 |
| rs1010777 | rs965513 | 0.354957 | 0.106491 | 0.000572 | 99807681 | 224 |
| rs987142 | rs965513 | 0.354957 | 0.106491 | 0.000572 | 99814467 | 225 |
| rs3780416 | rs965513 | 0.660153 | 0.106204 | 0.000337 | 99714386 | 226 |
| rs1610323 | rs965513 | 0.363691 | 0.104577 | 0.000777 | 99856447 | 227 |
| rs1588636 | rs965513 | 1 | 0.103112 | 0.000037 | 99577584 | 228 |
| rs3780459 | rs965513 | 0.377714 | 0.100458 | 0.001121 | 99948789 | 229 |

The markers were selected from the Caucasian HapMap dataset, using a cutoff of r2 greater than 0.1. Shown are marker names, anchor marker, values for D′ and r2 for the LD between the two markers, the corresponding P-value, position of the marker in NCBI Build 36 of the human genome assembly, and the identity of the SEQ ID for the flanking sequence of the marker.

### Example 2

In order to search for sequence variants conferring risk of thyroid cancer, we conducted a genome-wide association study (GWAS) with 192 histopathologically confirmed Icelandic thyroid cancer cases and 37,196 controls genotyped using the Illumina HumanHap300 and HumanCNV370-duo Bead Chip genotyping platform. Furthermore, we used a method where known genotypes of relatives are used to provide information on thyroid cancer cases not genotyped (in silico genotyping), in order to add genotypes that are equivalent to, on average per SNP, an additional 186 thyroid cancer patients (Gudbjartsson, D F et al Nat Genet. 40:609-15 (2008)). After removing SNPs that failed quality checks, a total of 304,083 SNPs were tested for association. We calculated the allelic odds ratio (OR) for each SNP assuming the multiplicative model and a standard likelihood ratio χ² statistic was computed for the purpose of testing. The results were adjusted for familial relatedness between individuals and for potential population stratification using the method of genomic control (Devlin B & Roeder K Biometrics 55:997-1004 (1999)); the χ² statistics were divided by an estimated inflation factor of 1.09.

We observed several strong signals located in the same linkage disequilibrium (LD) region as the Forkehead factor E1 (FOXE1) gene on 9q22.33 (FIG. 2; Table 3). In an attempt to confirm these results we proceeded to genotype these SNPs in additional 241 Icelandic thyroid cancer cases using Centaurus single track assay genotyping. Combining these results and the results from the GWAS, the strongest association signals were observed for allele A of rs965513 (rs965513-A) and allele A of rs10759944 (rs10759944-A) with an OR of 1.77 for both variants (P=6.8×10⁻²⁰ and P=1.7×10⁻¹⁹ for rs965513 and rs10759944, respectively) (Table 3 and Table 4). These two SNPs are nearly perfect surrogates of each other (r²=1 in the Utah CEPH (CEU) HapMap samples and r²=0.998 in the Icelandic samples) and since the effects of the variants cannot be distinguished from each other, we elected to focus on rs965513-A in subsequent investigations. Controlling for rs965513-A in a multivariate analysis, none of the remaining SNPs on 9q22.33 is significant.

We next tested the association of rs965513 to thyroid cancer in two case-control groups of European descent, with populations from Columbus, Ohio, United States (US) (342 cases and 384 controls) and Spain (90 cases and 1,343 controls). Association to rs965513 replicated in both study groups (Table 4). A test of heterogeneity in the ORs between the three study populations showed no significant difference (P=0.58 for rs965513). Combining the results from Iceland, Columbus and Spain gave an estimated OR of 1.75 for rs965513-A (P=1.7×10⁻²⁷).

In order to investigate the mode of inheritance, we computed the genotype-specific ORs and found that the multiplicative model provided an adequate fit for both variants (Table 5).

Approximately 11% of individuals in the general population are homozygous carriers of rs965513-A. Homozygous carriers of rs965513-A are estimated to have 3.1 fold greater risk, respectively, of developing the disease than non-carriers. Furthermore, we observed that the frequency of rs965513-A was higher among cases diagnosed at a younger age in all three populations. With the data combined, it is estimated that, for each allele carried, age at diagnosis is reduced by 2.42 years (P=0.0014) (Table 6).

We analyzed the effect of rs965513 in the four main histological classes of thyroid cancer. The majority of the Spanish and Icelandic sample collections consist of PTC (˜85%) and FTC (˜12%) and all of the cases from Columbus were PTC. For rs965513-A, the observed OR for PTC in the combined analysis of the three populations was 1.80 (P=4.7×10⁻²³) and for FTC the OR was 1.55, based on the Icelandic and Spanish samples only (P=0.016) (Table 7). This demonstrates that the variant affects the risk of the two main histological types of thyroid cancer. The numbers of other histological thyroid cancer types were too limited to draw meaningful conclusions.

The SNP rs965513 resides on 9q22.33 within a LD-region where the following genes have been localized: XPA, FOXE1, C9orf156 and HEMGN (FIG. 2). The closest gene is FOXE1, located about 57 kb telomeric to rs965513. FOXE1 is important for both pituitary- and thyroid gland formation (Dathan, N et al Dev Dyn 224:450456 (2002); De Felice, M et al Nat Genet 19:395-98 (1998)) and is at the center of a regulatory network of transcription factors and cofactors that initiate thyroid differentiation at the embryonic stage (Parlato R et al. Dev Biol 276:464-75 (2004)). Furthermore, mutations of the FOXE1 gene cause human syndromes that are associated with thyroid agenesis, among other phenotypes (De Felice, M et al Nat Genet. 19:395-98 (1998); Clifton-Bligh R J et al. Nat Genet 19:399-1401 (1998)). FOXE1 is also necessary for the maintenance of the differentiated state of the thyroid, based on its involvement in regulating the transcription of thyroid-specific genes, such as the thyroglobulin (Tg) and thyroperoxidase (TPO) genes. Regulated expression of both of these genes is pivotal for the synthesis of the thyroid hormones triiodothyronine (T₃) and thyroxine (T₄) as Tg is the precursor of the T₃ and T₄, and their synthesis is catalyses by TPO. Central to the thyroid hormone synthesis and secretion control is the thyroid stimulating hormone (TSH) that acts as principal regulator.

Given the involvement of FOXE1 in the biology of the thyroid gland, we assessed the effect of rs965513-A on circulating levels in serum of: TSH (N=12,035), free T₄ (N=7,108), and free T₃ (N=3,593). The data used came from series of measurements collected over a period of 11 years (from 1997 to 2008) from Icelanders not known to have thyroid cancer (Table 8). rs965513-A was associated with decreased serum levels of TSH by 5.9% per copy of rs965513-A (P=2.90×10⁻¹⁴; Table 9), and also with serum levels of T₃ and T₄, yet in opposite direction; with an increase in T₃ levels by 1.2% and a decrease in T₄ levels by 1.2% per copy of rs965513-A (P=3.00×10⁻³ and 6.10×10⁻⁵ for T₃ and T₄, respectively) (Table 9). These data demonstrate that the 9q22.33 variant affects some aspects of the endocrine function of the thyroid.

Taken together, the effect of rs965513 on 9q22.33 on thyroid and thyroid related hormones, the proximity of rs965513 to FOXE1, and the controlling effect of FOXE1 on thyroid specific genes, strongly suggests that the association between thyroid cancer and rs965513 is mediated through processes involving FOXE1. Furthermore, the expression of FOXE1 has been shown to be abnormal in thyroid tumors (Sequeira, M J et al. Thyroid 995-1001 (2001)). This variant is therefore likely to be among the most important determinants of genetic susceptibility to thyroid cancer.

### Methods

Subjects. Icelandic study population. Individuals diagnosed with thyroid cancer were identified based on a nationwide list from the Icelandic Cancer Registry (ICR) (http://www.krabbameinsskra.is/) that contained all 1,110 Icelandic thyroid cancer patients diagnosed from Jan. 1, 1955, to Dec. 31, 2007. Thereof 1.097 were non-medullary thyroid cancers. The Icelandic thyroid cancer study population consists of 460 patients (diagnosed from December 1974 to June 2007) recruited from November 2000 until April 2008, of whom 454 (98%) were successfully genotyped in this study. The histology of all thyroid carcinomas used in the present study has been reviewed and confirmed. A total of 192 patients were included in a genome wide SNP genotyping effort, using Illumina Sentrix HumanHap300 (n=96) and HumanCNV370-duo Bead Chip (n=96) microarrays (Illumina, San Diego, Calif., USA) and were successfully genotyped according to our quality control criteria and used in the present case-control association analysis. The remaining 241 cases were genotyped using the Centaurs single track genotyping platform. The mean age at diagnosis for the consenting patients was 44 years (median 43 years) and the range was from 13 to 87 years, while the mean age at diagnosis was 56 years for all thyroid cancer patients in the ICR. The median time from diagnosis to blood sampling was 10 years (range 0 to 46 years. When we compared the frequency of A-rs965513 between individuals diagnosed before 1998 and those diagnosed 1998 or later no significant difference was observed (P=0.97). The 37,202 controls (16,109 males (43.3%) and 21,093 females (56.7%)) used in this study consisted of individuals belonging to different genetic research projects at deCODE. The individuals have been diagnosed with common diseases of the cardio-vascular system (e.g. stroke or myocardial infraction), psychiatric and neurological diseases (e.g. schizophrenia, bipolar disorder), endocrine and autoimmune system (e.g. type 2 diabetes, asthma), malignant diseases (e.g. cancer of the breast or prostate) as well as individuals randomly selected from the Icelandic genealogical database. No single disease project represented more than 6% of the total number of controls. The controls had a mean age of 84 years and the range was from 8 to 105 years. A liner regression analysis showed no correlation between allele frequency of A-rs965513 and year of birth among the Icelandic controls (P>0.2). The controls were absent from the nationwide list of thyroid cancer patients according to the ICR. The DNA for both the Icelandic cases and controls was isolated from whole blood using standard methods.

The study was approved by the Data Protection Commission of Iceland and the National Bioethics Committee of Iceland. Written informed consent was obtained from all subjects. Personal identifiers associated with medical information and blood samples were encrypted with a third-party encryption system as previously described (Gulcher, J G et al. Eur J Hum Genet. 8:739-42 (2000)).

Columbus, Ohio, US. The study was approved by the Institutional Review Board of Ohio State University. All the subjects provide written informed consent. Cases (n=342) were histologically confirmed papillary thyroid carcinoma patients (including traditional PTC and follicular variant PTC). These patients were admitted to the Ohio State University Comprehensive Cancer Center, except one case was obtained through Cooperative Human Tissue Network (CHTN); this case was admitted to the University of Pennsylvania Medical Center. All cases are Caucasian; 92 men, 250 women, median age 40 years, range 13 to 88. The genomic DNA was extracted either from blood samples, or fresh frozen normal thyroid tissues from PTC patients. Controls (n=384) were individuals without clinically diagnosed thyroid cancers from central Ohio area. All controls are Caucasian, 143 men, 241 women, median age 51 years, range 18 to 94.

Spain. The Spanish study population consisted of 90 thyroid cancer cases. The cases were recruited from the Oncology Department of Zaragoza Hospital in Zaragoza, Spain, from October 2006 to June 2007. All patients were of self-reported European descent. Clinical information including age at onset, grade and stage was obtained from medical records. The average age at diagnosis for the patients was 48 years (median 49 years) and the range was from 22 to 79 years. The 1,343 Spanish control individuals 579 (43%) males and 764 (57%) females, who had a mean age of 51 (median age 50 and range 12-87 years) were approached at the University Hospital in Zaragoza, Spain, and were not known to have thyroid cancer. The DNA for both the Spanish cases and controls was isolated from whole blood using standard methods. Study protocols were approved by the Institutional Review Board of Zaragoza University Hospital. All subjects gave written informed consent.

### Statistical Analysis

Association analysis. A likelihood procedure described previously described (Gretarsdottir S et al. Nat Genet 35:131-38 (2003)) and implemented in the NEMO software was used for the association analyses. An attempt was made to genotype all individuals for the SNPs reported. The yield was higher than 95% for the SNPs in every group. We tested the association of an allele to thyroid cancer using a standard likelihood ratio statistic that, if the subjects were unrelated, would have asymptotically a χ² distribution with one degree of freedom under the null hypothesis. Allelic frequencies rather than carrier frequencies are presented for the markers in the main text. Allele-specific ORs and associated P values were calculated assuming a multiplicative model for the two chromosomes of an individual (Falk C T & Rubinstein P Ann Hum Genet 51(Pt 3):227-33 (1987)). For each of the three case-control groups there was no significant deviation from HWE in the controls (P>0.3). Results from multiple case-control groups were combined using a Mantel-Haenszel model (Mantel, N & Haenszel, W J Natl Cancer Inst 22:719-48 (1959)) in which the groups were allowed to have different population frequencies for alleles, and genotypes but were assumed to have common relative risks (see also Gudmundsson et al. Nat Genet 39:977-83 (2007)).

Correction for relatedness and genomic control. Some individuals in the Icelandic GWAS group were related to each other, causing the aforementioned χ² test statistic to have a mean >1. We estimated the inflation factor by using a method of genomic control (Devlin B. Roeder K. Biometrics 55:997-1004 (1999), calculating the average of the 304,083 χ² statistics. According to this method the inflation factor was estimated to be 1.09. Based on the change in sample size of genotyped and in-silico genotyped cases due to single assay genotyping we estimated the inflation factor in the combined Icelandic sample set to be 1.12. The χ² statistics for the test for association with thyroid cancer in the combined Icelandic samples were adjusted accordingly.

### Genotyping

Illumina genotyping. 192 and 37,202 Icelandic case- and control-samples respectively, were assayed with either the Illumina Sentrix HumanHap300 or the HumanCNV370-duo Bead Chips (Illumina, San Diego, Calif., USA) and were successfully genotyped according to our quality control criteria. Of the SNPs assayed on the chip, SNPs that had yield lower than 95%, had a minor allele frequency below 0.01 in the combined set of cases and controls, or were monomorphic were omitted from the analysis. An additional 4,632 SNPs showed a significant distortion from Hardy-Weinberg equilibrium in the controls (P<1.0×10⁻³). In total, 13,420 unique SNPs were removed from the study. Thus, the analysis reported in the main text utilizes 304,083 SNPs. Any samples with a call rate below 98% were excluded from the analysis.

Single track assay SNP genotyping. Single SNP genotyping for the two case-control groups from Iceland and Spain was carried out by deCODE Genetics in Reykjavik, Iceland, applying the Centaurus (Nanogen) platform (Kutyavin, I V et al Nucleic Acids Res 34:e128 (2006)). The quality of each Centaurus SNP assay was evaluated by genotyping each assay in the CEU and/or YRI HapMap samples and comparing the results with the HapMap publicly released data. Assays with >1.5% mismatch rate were not used and a linkage disequilibrium (LD) test was used for markers known to be in LD. We genotyped 330 individuals using both the Illumina Hap300 chip and Centaurus single track SNP assay and observed a mismatch rate lower than 0.5%.

Genotyping of samples from the Ohio study populations was done using the SNaPshot (PE Applied Biosystems, Foster City, Calif.) genotyping platform at the Ohio State University, as previously described (He H. et al. Thyroid 15:660-667 (2005)).

### TSH, Free-T₄ and Free-T₃ Measurements.

TSH, free-T₄ and free-T₃ levels were measured for Icelanders seeking medical care between the years 1997 and 2008 at the Iceland Medical Center (Laeknasetrid), a clinic specializing in internal medicine. The measurements were performed in the Laboratory in Mjodd, Reykjavik, Iceland. Measurements outside the specified range were discarded. The log-transformed measurements were adjusted for sex and age at measurement using a generalized additive model. In the case when multiple measurements were available for a single individual the mean of the log-adjusted measurements was used in subsequent analyses. The age and sex adjusted log-transformed measurement were regressed on allele counts using classical linear regression.

TABLE 3
Association result for Icelandic thyroid cancer patients from GWAS and replication study in Iceland only.

| | | | | Results from genome-wide association study^{a} | | | | | |
|---|---|---|---|---|---|---|---|---|---|
| | | Chromo- | Location | Cases | Controls | Frequency | | | P |
| Marker | Allele | some | (Mb) | (n) | (n) | Cases | Controls | OR (95% c.i.) | value^{b} |
| rs965513 | A | 9 | 97.636 | 378 | 37,196 | 0.484 | 0.352 | 1.73 (1.49, 2.01) | 7.5E−13 |
| rs10759944 | A | 9 | 97.637 | 378 | 37,146 | 0.485 | 0.352 | 1.74 (1.49, 2.02) | 6.2E−13 |
| rs907580 | A | 9 | 97.702 | 378 | 37,154 | 0.388 | 0.281 | 1.62 (1.38, 1.89) | 1.8E−09 |
| rs10984103 | A | 9 | 97.719 | 378 | 37,197 | 0.465 | 0.359 | 1.55 (1.33, 1.80) | 1.5E−08 |
| rs925487 | G | 9 | 97.716 | 378 | 37,153 | 0.464 | 0.359 | 1.55 (1.33, 1.80) | 1.7E−08 |
| rs7024345 | A | 9 | 97.675 | 378 | 37,176 | 0.388 | 0.285 | 1.59 (1.36, 1.86) | 6.4E−09 |
| rs1443434 | G | 9 | 97.697 | 377 | 37,106 | 0.483 | 0.385 | 1.49 (1.28, 1.73) | 2.6E−07 |

| | Combined results from GWAS and replication single track assay genotyping^{a} | | | | | |
|---|---|---|---|---|---|---|
| | Cases | Controls | Frequency | | | P |
| Marker | (n) | (n) | Cases | Controls | OR (95% c.i.) | value^{b} |
| rs965513 | 579 | 37,196 | 0.490 | 0.352 | 1.77 (1.57, 2.00) | 6.8E−20 |
| rs10759944 | 571 | 37,146 | 0.490 | 0.352 | 1.77 (1.57, 2.01) | 1.7E−19 |
| rs907580 | 571 | 37,154 | 0.395 | 0.281 | 1.66 (1.46, 1.89) | 1.1E−14 |
| rs10984103 | 574 | 37,197 | 0.472 | 0.359 | 1.59 (1.41, 1.81) | 2.2E−13 |
| rs925487 | 571 | 37,153 | 0.472 | 0.359 | 1.60 (1.41, 1.81) | 2.6E−13 |
| rs7024345 | 577 | 37,176 | 0.387 | 0.285 | 1.58 (1.39, 1.80) | 1.9E−12 |
| rs1443434 | 446 | 37,106 | 0.488 | 0.385 | 1.52 (1.32, 1.74) | 2.8E−09 |

^{a}Included are individuals with genotypes from an in-silico analysis.
^{b}Results were adjusted as described in main text.

TABLE 4
Association results for rs965513 and thyroid cancer
in Iceland, Spain and the United States

| Study population | | | | |
|---|---|---|---|---|
| (n cases/n | | | | |
| controls) | Frequency | | | |
| Variant (allele) | Cases | Controls | OR (95% c.i.) | P value |
| Iceland genome- | 0.484 | 0.352 | 1.73 (1.49, 2.01) | 7.5 × 10⁻¹³ |
| wide scan | | | | |
| (378^{a}/37,196) | | | | |
| Iceland all | 0.490 | 0.352 | 1.77 (1.57, 2.00) | 6.8 × 10⁻²⁰ |
| (579^{b}/37,196) | | | | |
| Columbus, Ohio, | 0.471 | 0.329 | 1.81 (1.45-2.26) | 1.2 × 10⁻⁷ |
| US (294/384) | | | | |
| Spain (89/1,343) | 0.444 | 0.342 | 1.54 (1.13-2.09) | 6.5 × 10⁻³ |
| Combined | — | 0.336 | 1.72 (1.43, 2.05) | 3.7 × 10⁻⁹ |
| Columbus and | | | | |
| Spain (383/1,727) | | | | |
| All combined | — | 0.341 | 1.75 (1.59, 1.94) | 1.7 × 10⁻²⁷ |
| (962/38,923)^{c} | | | | |

Shown are the corresponding numbers of cases and controls (n), allelic frequencies of variants in affected and control individuals, the allelic odds-ratio (OR) with 95% confidence interval (95% c.i.) and P values based on the multiplicative model. All P values shown are two-sided.
^{a}The Icelandic genome-wide case study population is made up of individuals with genotypes from the Illumina Hap300/370 chips (n = 192) and individuals with genotypes from in-silico analysis (n = 186 on average per marker).
^{b}The combined Icelandic all study population is comprised of individuals with genotypes from the Illumina Hap300/370 chips and individuals with genotypes from single track assay genotyping (n = 454) as well as individuals with genotypes from in-silico analysis (n = 125 on average per marker). Icelandic controls were genotyped using the Illumina Hap300/370 chips.
^{c}For the combined study populations, the reported control frequency was the average, unweighted control frequency of the individual populations, while the OR and the P value were estimated using the Mantel-Haenszel model.

TABLE 5
Model-free estimates of the genotype relative risks of rs965513 (A)

| Study group | | Genotype | | | |
|---|---|---|---|---|---|
| Variant (allele) | Allelic | relative risk^{a} | | | P |
| (n case/n controls) | OR | 00 | 0X | XX | value^{b} |
| Iceland (439/37,196) | 1.84 | 1 | 1.55 | 3.37 | 0.075 |
| Columbus, Ohio, US (294/384) | 1.81 | 1 | 1.65 | 3.32 | 0.51 |
| Spain (89/1,343) | 1.54 | 1 | 1.74 | 2.28 | 0.38 |

^{a}Genotype relative risks for heterozygous-(0X) and homozygous carriers (XX) compared with risk for non-carriers (00).
^{b}Test of the multiplicative model versus the full model, one degree of freedom

TABLE 6
Association analysis of rs965513-A for a) gender and b) age at diagnosis.
a

| | | OR |
|---|---|---|
| Study population | | males vs. females |
| (n males/n females) | P value | (95% c.i.) |
| Iceland (105/334) | 0.97 | 1.01 (0.74, 1.37) |
| Columbus, Ohio, US (72/222) | 0.089 | 1.39 (0.95, 2.03) |
| Spain (20/69) | 0.42 | 1.34 (0.66, 2.71) |
| All combined (197/625) | 0.19 | 1.16 (0.93, 1.46) |

b

| Study population | | |
|---|---|---|
| (n individuals with age | | Effect on age at |
| informaton) | P value | diagnosis (years) |
| Iceland (439) | 0.077 | −1.87 (−3.94, +0.20) |
| Columbus, Ohio, US (292) | 0.13 | −1.88 (−4.30, +0.55) |
| Spain (89) | 0.0029 | −6.64. (−11.0, −2.27) |
| All combined (820) | 0.0014 | −2.42 (−3.90, −0.94) |

All P values shown are two-sided.
(a) Shown is the allelic odds-ratio (OR) with 95% confidence interval (95% c.i.) and P values based on an association analysis comparing the frequency of the relevant risk variant in males vs. females.
(b) Shown is the effect on age at diagnosis (in years) with 95% c.i. of each allele carried of the risk allele (rs965513-A). The minus sign (“−”) denotes a decrease and the plus sign (“+”) an increase in age at diagnosis.

TABLE 7
Association results in Iceland, Spain and USA for different thyroid carcinoma histological types

| Carcinoma type | | | | Cases | Controls | Frequency | |
|---|---|---|---|---|---|---|---|
| Marker (allele) | Study population | P value | OR (95% c.i.) | (n) | (n) | Cases | Controls |
| Papillary | | | | | | | |
| rs965513 (A) | Iceland | 2.22 × 10⁻¹⁶ | 1.88 (1.61, 2.18) | 368 | 37,194 | 0.504 | 0.352 |
| rs965513 (A) | Spain | 0.036 | 1.43 (1.02, 2.01) | 76 | 1,343 | 0.427 | 0.342 |
| rs965513 (A) | Columbus, Ohio | 1.19 × 10⁻⁷ | 1.81 (1.45, 2.26) | 294 | 384 | 0.471 | 0.329 |
| rs965513 (A) | All combined | 4.70 × 10⁻²³ | 1.80 (1.60, 2.02) | 738 | 38,537 | — | 0.341 |
| Follicular | | | | | | | |
| rs965513 (A) | Iceland | 0.067 | 1.43 (0.97, 2.10) | 55 | 37,194 | 0.436 | 0.352 |
| rs965513 (A) | Spain | 0.058 | 2.35 (0.97, 5.70) | 10 | 1,343 | 0.550 | 0.342 |
| rs965513 (A) | All combined | 0.016 | 1.55 (1.09, 2.20) | 65 | 38,537 | — | 0.347 |

All P values shown are two-sided. Shown are the corresponding numbers of cases and controls (N), allelic frequencies of variants in affected and control individuals, the allelic odds-ratio (OR) with 95% confidence interval (95% c.i.) and P values based on the multiplicative model.
For the combined study populations, the reported control frequency was the average, unweighted control frequency of the individual populations, while the OR and the P value were estimated using the Mantel-Haenszel model.

TABLE 8
An overview of the TSH, free-T₄ and free-T₃ measurements available.

| | | | | Individuals with | | Individuals not |
|---|---|---|---|---|---|---|
| | | Individuals with | Measurements | thyroid cancer | | with cancer and |
| Measurement | | measurement | per patient^{a} | and measurement | Range | inside range |
| type | Units | (N) | (N) | (N) | used | (N) |
| TSH | mlU/L | 25,660 | 1.9 | 302 | 0.1-10.0 | 25,099 |
| Free-T4 | pmol/L | 14,887 | 1.7 | 294 | 8.4-333.4 | 14,568 |
| Free-T3 | pmol/L | 7,433 | 1.5 | 147 | 2.6-12.5 | 7,250 |

^{a}The geometric mean of the number of measurements per patient.

TABLE 9
Association results for rs965513 and levels of thyroid
related hormones in Icelandic individuals

| Type of | Individuals | Effect per risk allele | |
|---|---|---|---|
| measurement | (n) | (95% c.i.) | P value |
| Thyroid | 12,035 | −5.9% (−7.4%, −4.4%) | 2.9 × 10⁻¹⁴ |
| stimulating | | | |
| hormone (TSH) | | | |
| Free thyroxine | 7,108 | −1.2% (−1.8%, −0.6%) | 6.1 × 10⁻⁵ |
| (T₄) | | | |
| Free | 3,593 | +1.2% (+0.4%, +2.0%) | 3.0 × 10⁻³ |
| triiodothyronine | | | |
| (T₃) | | | |

Shown are association results (per risk allele) for individuals (n) with a given type of measurement and a known carrier status for rs965513. The minus sign (“−”) denotes a decreased and the plus sign (“+”) an increased concentration of thyroid related hormones.

1. A method for determining a susceptibility to thyroid cancer in a human individual, comprising determining whether at least one allele of at least one polymorphic marker is present in a nucleic acid sample obtained from the individual, wherein the at least one polymorphic marker is selected from the group consisting of rs965513, and markers in linkage disequilibrium therewith, wherein the linkage disequilibrium is characterized by a value for r² of at least 0.2, and
determining a susceptibility to thyroid cancer in the subject from the presence or absence of the at least one allele, wherein the presence of the at least one allele is indicative of a susceptibility to thyroid cancer for the individual.

2. The method according to claim 1, wherein the at least one polymorphic marker is selected from the group consisting of the markers set forth in Table 2.

3. The method according to claim 2, wherein the at least one polymorphic marker is selected from the group consisting of rs965513, rs10759944, rs907580, rs10984103, rs925487, rs7024345 and rs1443434.

4. The method according to claim 1, further comprising assessing the frequency of at least one haplotype in the individual.

5. The method of claim 4, wherein the susceptibility conferred by the presence of the at least one allele or haplotype is increased susceptibility.

6. The method according to claim 5, wherein the presence of allele A in marker rs965513, allele A in marker rs907580, allele A in marker rs10759944, allele A in marker rs10984103, allele G in marker rs925487, allele A in marker rs7024345, and allele G in marker rs1443434 is indicative of increased susceptibility to thyroid cancer in the individual.

7. The method according to claim 5, wherein the presence of the at least one allele or haplotype is indicative of increased susceptibility to thyroid cancer with a relative risk (RR) or odds ratio (OR) of at least 1.6.

8. The method according to claim 5, wherein the presence of the at least one allele or haplotype is indicative of increased susceptibility with a relative risk (RR) or odds ratio (OR) of at least 1.7.

9. The method according to claim 1, wherein the susceptibility conferred by the presence of the at least one allele or haplotype is decreased susceptibility.

10. The method of claim 1, further comprising determining whether at least one at-risk allele of at least one at-risk variant for thyroid cancer not in linkage disequilibrium with any one of the markers set forth in Table 2 is present in a sample comprising genomic DNA from a human individual or a genotype dataset derived from a human individual.

11. The method of claim 1, comprising determining whether at least one allele in each of at least two polymorphic markers is present in a sample comprising genomic DNA from a human individual, wherein the presence of the at least one allele in the at least two polymorphic markers is indicative of an increased susceptibility to thyroid cancer.

12. A method of determining a susceptibility to thyroid cancer in a human individual, the method comprising:
obtaining nucleic acid sequence data about a human individual identifying at least one allele of at least one polymorphic marker selected from the group consisting of rs965513 (SEQ ID NO: 1), and markers in linkage disequilibrium therewith, wherein the linkage disequilibrium is characterized by a value for r² of at least 0.2, and wherein different alleles of the at least one polymorphic marker are associated with different susceptibilities to thyroid cancer in humans, and
determining a susceptibility to thyroid cancer from the nucleic acid sequence data.

13. The method of claim 12, comprising obtaining nucleic acid sequence data about at least two of said polymorphic markers selected from the group consisting of rs965513 (SEQ ID NO: 1), and markers in linkage disequilibrium therewith.

14. The method of claim 12, wherein determination of a susceptibility comprises comparing the nucleic acid sequence data to a database containing correlation data between the at least one polymorphic marker and susceptibility to thyroid cancer.

15. The method of claim 14, wherein the database comprises at least one risk measure of susceptibility to thyroid cancer for the at least one polymorphic marker.

16. The method of claim 14, wherein the database comprises a look-up table containing at least one risk measure of the at least one condition for the at least one polymorphic marker.

17. The method of claim 12, wherein obtaining nucleic acid sequence data comprises obtaining a biological sample from the human individual and analyzing sequence of the at least one polymorphic marker in nucleic acid in the sample.

18. The method of claim 17, wherein analyzing sequence of the at least one polymorphic marker comprises determining the presence or absence of at least one allele of the at least one polymorphic marker.

19. (canceled)

20. The method of claim 1, further comprising reporting the susceptibility to at least one entity selected from the group consisting of the individual, a guardian of the individual, a genetic service provider, a physician, a medical organization, and a medical insurer.

21. The method of claim 12, wherein the at least one polymorphic marker is selected from the group consisting of the markers listed in Table 2.

22. The method of claim 21, wherein the at least one polymorphic marker is selected from the group consisting of rs965513, rs10759944, rs907580, rs10984103, rs925487, rs7024345 and rs1443434.

23. The method of claim 1, wherein the at least one polymorphic marker is associated with the FoxE1 gene.

24. A method of identification of a marker for use in assessing susceptibility to thyroid cancer, the method comprising:
a. identifying at least one polymorphic marker in linkage disequilibrium with at least one marker selected from the group consisting of rs965513, rs10759944, rs907580, rs10984103, rs925487, rs7024345 and rs1443434, wherein the linkage disequilibrium is characterized by a value for r² of at least 0.2;
b. determining the genotype status of a sample of individuals diagnosed with, or having a susceptibility to, thyroid cancer; and
c. determining the genotype status of a sample of control individuals; and
d. identifying the at least one polymorphic marker for use in assessing susceptibility to thyroid cancer from (b) and (c),
wherein a significant difference in frequency of at least one allele in at least one polymorphism in individuals diagnosed with, or having a susceptibility to, thyroid cancer, as compared with the frequency of the at least one allele in the control sample is indicative of the at least one polymorphism being useful for assessing susceptibility to thyroid cancer.

25. The method according to claim 24, wherein an increase in frequency of the at least one allele in the at least one polymorphism in individuals diagnosed with, or having a susceptibility to, thyroid cancer, as compared with the frequency of the at least one allele in the control sample is indicative of the at least one polymorphism being useful for assessing increased susceptibility to thyroid cancer.

26. The method according to claim 24, wherein a decrease in frequency of the at least one allele in the at least one polymorphism in individuals diagnosed with, or having a susceptibility to, thyroid cancer, as compared with the frequency of the at least one allele in the control sample is indicative of the at least one polymorphism being useful for assessing decreased susceptibility to, or protection against, thyroid cancer.

27. A method of genotyping a nucleic acid sample obtained from a human individual comprising determining whether at least one allele of at least one polymorphic marker is present in a nucleic acid sample from the individual sample, wherein the at least one marker is selected from the group consisting of rs965513, and markers in linkage disequilibrium therewith, wherein the linkage disequilibrium is characterized by a value for r² of at least 0.2, and wherein determination of the presence of the at least one allele in the sample is indicative of a susceptibility to thyroid cancer in the individual.

28. The method according to claim 27, wherein determination of the presence of allele A in marker rs965513, allele A in marker rs907580, allele A in marker rs10759944, allele A in marker rs10984103, allele G in marker rs925487, allele A in marker rs7024345, and allele G in marker rs1443434 is indicative of increased susceptibility of thyroid cancer in the individual.

29. The method according to claim 27, wherein genotyping comprises amplifying a segment of a nucleic acid that comprises the at least one polymorphic marker by Polymerase Chain Reaction (PCR), using a nucleotide primer pair flanking the at least one polymorphic marker.

30. The method according to claim 27, wherein genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, single-stranded conformation analysis and micro array technology.

31. The method according to claim 30, wherein the process comprises allele-specific probe hybridization.

32. The method according to claim 30, wherein the process comprises a microarray technology.

33. The method according to claim 27, comprising:
1) contacting copies of the nucleic acid with a detection oligonucleotide probe and an enhancer oligonucleotide probe under conditions for specific hybridization of the oligonucleotide probe with the nucleic acid;
wherein
a) the detection oligonucleotide probe is from 5-100 nucleotides in length and specifically hybridizes to a first segment of a nucleic acid whose nucleotide sequence is given by any one of SEC) ID NO: 1-229;
b) the detection oligonucleotide probe comprises a detectable label at its 3′ terminus and a quenching moiety at its 5′ terminus;
c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
d) a single base gap exists between the first segment and the second segment, such that when the oligonucleotide probe and the enhancer oligonucleotide probe are both hybridized to the nucleic acid, a single base gap exists between the oligonucleotides;
2) treating nucleic acid with an endonuclease that will cleave the detectable label from the 3′ terminus of the detection probe to release free detectable label when the detection probe is hybridized to the nucleic acid; and
3) measuring free detectable label, wherein the presence of the free detectable label indicates that the detection probe specifically hybridizes to the first segment of the nucleic acid, and indicates the sequence of the polymorphic site as the complement of the detection probe.

34.-37. (canceled)

38. The method of claim 1, further comprising analyzing non-genetic information to make risk assessment, diagnosis, or prognosis of the individual.

39. The method of claim 38, wherein the non-genetic information is selected from age, gender, ethnicity, socioeconomic status, previous disease diagnosis, medical history of subject, family history of thyroid cancer, biochemical measurements, and clinical measurements.

40. The method of claim 1, further comprising calculating combined risk.

41.-47. (canceled)

48. A computer-readable medium having computer executable instructions for determining susceptibility to thyroid cancer in a human individual, the computer readable medium comprising:
data indicative of at least one polymorphic marker;
a routine stored on the computer readable medium and adapted to be executed by a processor to determine risk of developing thyroid cancer in an individual for the at least one polymorphic marker;
wherein the at least one polymorphic marker is selected from the group consisting of rs965513, and markers in linkage disequilibrium therewith, wherein the linkage disequilibrium is characterized by a value for r² of at least 0.2.

49. The computer readable medium of claim 48, wherein the computer readable medium contains data indicative of at least two polymorphic markers.

50. The computer readable medium of claim 48, wherein the data indicative of at least one polymorphic marker comprises parameters indicative of susceptibility to thyroid cancer for the at least one polymorphic marker, and wherein risk of developing thyroid cancer in an individual is based on the allelic status for the at least one polymorphic marker in the individual.

51. The computer readable medium of claim 48, wherein said data indicative of at least one polymorphic marker comprises data indicative of the allelic status of said at least one polymorphic marker in the individual.

52. The computer readable medium of claim 48, wherein said routine is adapted to receive input data indicative of the allelic status of said at least one polymorphic marker in said individual.

53. The computer readable medium of claim 48, wherein the at least one polymorphic marker is selected from the markers set forth in Table 2.

54. The computer-readable medium of claim 48, wherein the at least one polymorphic marker is selected from the group consisting of rs965513, rs10759944, rs907580, rs10984103, rs925487, rs7024345 and rs1443434.

55. The computer readable medium of claim 48, comprising data indicative of at least one haplotype comprising two or more polymorphic markers.

56. An apparatus for determining a genetic indicator for thyroid cancer in a human individual, comprising:
a processor
a computer readable memory having computer executable instructions adapted to be executed on the processor to analyze marker and/or haplotype information for at least one human individual with respect to at least one polymorphic marker selected from the group consisting of rs965513, and markers in linkage disequilibrium therewith, wherein the linkage disequilibrium is characterized by a value for r² of at least 0.2, and
generate an output based on the marker or haplotype information, wherein the output comprises a risk measure of the at least one marker or haplotype as a genetic indicator of thyroid cancer for the human individual.

57. The apparatus according to claim 56, wherein the computer readable memory further comprises data indicative of the frequency of at least one allele of at least one polymorphic marker or at least one haplotype in a plurality of individuals diagnosed with thyroid cancer, and data indicative of the frequency of at the least one allele of at least one polymorphic marker or at least one haplotype in a plurality of reference individuals, and wherein a risk measure is based on a comparison of the at least one marker and/or haplotype status for the human individual to the data indicative of the frequency of the at least one marker and/or haplotype information for the plurality of individuals diagnosed with thyroid cancer.

58. The apparatus according to claim 56, wherein the computer readable memory further comprises data indicative of the risk of developing thyroid cane associated with at least one allele of at least one polymorphic marker or at least one haplotype, and wherein a risk measure for the human individual is based on a comparison of the at least one marker and/or haplotype status for the human individual to the risk of thyroid cancer associated with the at least one allele of the at least one polymorphic marker or the at least one haplotype.

59. The apparatus according to claim 56, wherein the computer readable memory further comprises data indicative of the frequency of at least one allele of at least one polymorphic marker or at least one haplotype in a plurality of individuals diagnosed with thyroid cancer, and data indicative of the frequency of at the least one allele of at least one polymorphic marker or at least one haplotype in a plurality of reference individuals, and wherein risk of developing thyroid cancer is based on a comparison of the frequency of the at least one allele or haplotype in individuals diagnosed with thyroid cancer and reference individuals.

60. The apparatus according to claim 56, wherein the at least one marker or haplotype comprises at least one marker selected from the group of markers set forth in Table 2.

61. The apparatus according to claim 56, wherein the risk measure is characterized by an Odds Ratio (OR) or a Relative Risk (RR).

62.-65. (canceled)


SEQUENCE LISTING (229 sequences)

SEQ ID NO: 1 - DNA, 599 bp, Homo sapiens
caaatatgaaggaagaagagtggaatacaggaattagaatacttgaaatagagtagttatgatgatgaaccacagaaatttagctggttaaggaaggaagtgagaacctgagggaaatgacagatagtgggaaaagtggaagggccagtgtattggaagtcttgatgaggtggattggaaatactgccccaagggagctgggatgaaaggaggtagcaggcagagaatgtaggtttttggtgatggtatggtcatggacatgacaaaggtaatgagtggctggaatggaacagatcaaargagtaaattaaaaagaagatgtattagtctgttctcacaatgctaacaaagacataacctgagcctgggtaatttataaaagaaagagatttaattgactcgcagttcagcatggctggggaggcctcaggaaacttacaatcatggtggaatgggaagccaacacgtacttcttcacatggtagcaggaagcaaaagtgctgagcaaaagggggaaaaggcccttataaaaccatcagatcttgtgagaactcactcattatcaccagaacagtatgagggtaactgtccccatgatt

SEQ ID NO: 2 - DNA, 599 bp, Homo sapiens
tgcttaccccaagtcacatagcaagttaggcaggagactgggcacaagacctcctgacttccgcaccaccacaaccctcaaattttttttaatgagtgtttttctttaaaacatagtatgaacactggaacagagggaaatgcagtattccaatttccatccacagtgttgtccgacctggcatctcactggagtccccaagcacaggccctggcagcagaccaggctgggttcaagtcctactcctgccttttgctagccccacggctttggcaaattactgcagttggaattattaastgttggaattattaaattgttggaattattaagtgaactaatgggcacagacattttgttccacacttagcatatagtaagcacccaattattgctattgccattagcagctccggcacttacctgccaggtgtgcaaggccctcaactgctaagccttctgtctcaaatgagacatcaccaagatttctcctaactccagagccctgtagtctccaccacctggcccaaatgaaacgatggaccccagaatcagggaagtatggtgggaacctgtgtcacttccctgtttctctttct

SEQ ID NO: 3 - DNA, 599 bp, Homo sapiens
ggcctccaatatggctcagcacagccctgatagaaaatattttgaaattttgcttgtatgaaatgtctctattctattctttcatttagctgggaattgaattctaggtcaaaaataattttccttcagaattttggaggcaatgttccatcatcttctcacttccagtgttactttctgagaagtcttcaatcactataattgctgaccatttttgtacgtgacctggttttgctgtttggaacctcatagaatcttttctttatcaccagtattcttgtactttgcagtaattgtttkgtggagctttttgtttgtttgtttgttttttaatctatggacctgattctcaaggattctttcattctggagacttatgcccttgggttctggagaaatcaatctctttttaaagaaaataatttttcaccttctttttttcactgtctggaagtactattagtcaaatttactgaattcgtccttatttttcttacattttctccactattttcatctctttgccatttgttttctattttcggacagactgccttgttgataagaggataagtgcagtctttcagatacattttctt

SEQ ID NO: 4 - DNA, 599 bp, Homo sapiens
attattttgcactaatttttatattttaaaatgctgtttaaaatatcatttattgtgattattgagattttgggtgctctcttgcattttgcacttgaagcaagtgcctcactttcctttctcttgtcccatccttaattcccatgacctttcttgggaagttactgaaatatgtgcttccctcaaaaaaggagaaaatcaagaaggagaaagacatggacacaagggcaaggcatgaaacacggcagaaagatcaagggaattcctgtagtgttggtgaagggtgatcctagggtgatraccatgcccaaatcagagaggccaagagacctagattgcagcaagacagaaggcaagaagtggaaactggtaagttcatacgaaaacacagagaggagggcattaaggatggtaataattttttttaattcagcaaatgaaaacacagataattattatctccagacaaaaccaaaagtccaaaaatggaaataaataaataaataaataaataaataaataaatagtattctactagttcagctgtgaaaagcatttagataatactataaacactgactattgatctaattaacatt

SEQ ID NO: 5 - DNA, 599 bp, Homo sapiens
tactgtattatgtacttgaaaattactgagagggtagatctcaagtgttttcatcacacacacacatacacacacacacacacacacacacacacacacacacacaaatgtaaccacatgaggtgatgaatatattaattagcttgattgtggtaatcatttcacaatgtatatacacatcaaaacatcatgttgtacaccttaaatatatacaatttttatttgccaattatatctcaattgttaaaaatcaagtaatagcaagttaattgagttatatagagatgtggaagaaaataytaaaaaagtgaaaagaggtggaaaaaatttgcttttgggtgggggatatgggagaaggcaaagcaatagggctactctttttgtgtcaaatcttacagagttatttgatctttaaactatgtatctgtataactttaaaaaatataaaaactttctaaaaacgatgtattgatagctgttttcagattagttgaaacatttgggaagaattagagaaaactacctagatgactcatccaagcaattattaagtccaggaaaaacaaaaagaagtacaaaaagagagacatagtaatatg

SEQ ID NO: 6 - DNA, 599 bp, Homo sapiens
tcaggtcaaagcagcaaggctttttctgacttagcctcagaagtcatacaacatcacttctgccatattctttttattacaagtgagagttccaggattggtccaggtccaagggaagggtagtacacatacacgtgaacactgggaggcatagtttattgagaccctcttacagctacctggagcatagagtcagaagtcttcctagcttctctgattgtactcgaccccttctccccgctatgcctctgaccatcctcttaattttggctgggagttgttgcagaagacacatccatrctccaggtgtgcacagtccccttcgtggtcagcagaatgtgtacagcctcattgtgagcagggccaacagtaggttaccaagctcctgcaatgctggagtgcccattaccaactcctgccaacccactgccacatagtatctcatgggctcttgtttaagctgaacatattcactggggccaccttggagaacttggccttgacctgcttaaacacttgagcaaggttctattctgagagcttggatctttcctgagaccaacaggactcacaggggttttgcagatgggactaagagc

SEQ ID NO: 7 - DNA, 599 bp, Homo sapiens
gtgggttggcaggagttggtaatgggcactccagcattgcaggagcttggtaacctactgttggccctgctcacaatgaggctgtacacattctgctgaccacgaaggggactgtgcacacctggagcatggatgtgtcttctgcaacaactcccagccaaaattaagaggatggtcagaggcatagcggggagaaggggtcgagtacaatcagagaagctaggaagacttctgactctatgctccaggtagctgtaagagggtctcaataaactatgcctcccagtgttcacgtgtatrtgtactacccttcccttggacctggaccaatcctggaactctcacttgtaataaaaagaatatggcagaagtgatgttgtatgacttctgaggctaagtcagaaaaagccttgctgctttgacctgagtctcttggaacattctctaaggagccgtgaactactatataagaagtctgccatactggggaagccaagtgtaggcactccggttaacagtccccgcacagctccgccttccagccattcccactaagacatcacacatacaagggaagttgtctcggatcctccagatca

SEQ ID NO: 8 - DNA, 599 bp, Homo sapiens
atcagagaagctaggaagacttctgactctatgctccaggtagctgtaagagggtctcaataaactatgcctcccagtgttcacgtgtatgtgtactacccttcccttggacctggaccaatcctggaactctcacttgtaataaaaagaatatggcagaagtgatgttgtatgacttctgaggctaagtcagaaaaagccttgctgctttgacctgagtctcttggaacattctctaaggagccgtgaactactatataagaagtctgccatactggggaagccaagtgtaggcactcyggttaacagtccccgcacagctccgccttccagccattcccactaagacatcacacatacaagggaagttgtctcggatcctccagatcagcccatccacaagctgaatactcccaagtgacttcagccagtaccacacagagcagaagaatctctcagctgatcctggcccaaattcctgacccacaaaattataatacaaaataaaatggttgctattttaagccactaagtgagggggcaattaatatgcagcaatacataaccagcatatcacctttgggagatgcttgagaata

SEQ ID NO: 9 - DNA, 599 bp, Homo sapiens
tcaggaggctgaggtggaagaatcccttgaactcaggaggaggaggttgcagtgagccgagattgcaccactgcactccagcctgggtgaaagagagagaagagattctgtctcacaaaaaaaaaaaaaaaaaaaaaaaaaagtgccgggcacggtggctcacgcctgtaatcccaacactttgggaagctgaggctgttggatcacttgaggtctggagttcgagaccagtctagccaacatggtaaaacccatgtctactaaaaatacaaaaattagccagttgtgatggcacatgcytaacatgcctgtaatcccaggtacttgggagactgaggcaggaaaattgcttgaacccaggaagtggagtttgcagtgagccaagatcgcaccaccgcactccagcctgggtgaaagaatgagaccctgcctcaaaaaaaaaaaaaaaaaaaaaaaggaaacaagagatatgtcaaccaaattcaatgtgtatatcagaatccacaaagtctgtggattctgatatatacattagtgtgtgtgtgtgtgtgtgtgtgtgtatttgaacatagactaggtatttagtgatattaacaatt

SEQ ID NO: 10 - DNA, 599 bp, Homo sapiens
cccaacactttgggaagctgaggctgttggatcacttgaggtctggagttcgagaccagtctagccaacatggtaaaacccatgtctactaaaaatacaaaaattagccagttgtgatggcacatgcttaacatgcctgtaatcccaggtacttgggagactgaggcaggaaaattgcttgaacccaggaagtggagtttgcagtgagccaagatcgcaccaccgcactccagcctgggtgaaagaatgagaccctgcctcaaaaaaaaaaaaaaaaaaaaaaaggaaacaagagatatrtcaaccaaattcaatgtgtatatcagaatccacaaagtctgtggattctgatatatacattagtgtgtgtgtgtgtgtgtgtgtgtgtatttgaacatagactaggtatttagtgatattaacaattatttttaattttaggtgtgatataatattgtgtatattttttaaagataaaagatgtatttagagataaaaagatatatgcatcccaggtatatctgggatttgcttcaaatgaatgcagtggtaagggggtaggaatagacagggtataaggaggcaagattggccatatg

SEQ ID NO: 11 - DNA, 599 bp, Homo sapiens
ggatgcataatggaggggagtttggagagggaacaaaattattaaggaggattatactgtcaccactcgacacaatggagactgctgcttttaactgccttaaacaagtcttgcttcataccagagctgcgactcaaggaggcgtcgccatgggaagtggatcctgcccagccgatcaaatctttcttcttggctgggtctttgagtgcaggttttctgcctatacaacaagagaatggatgtggctcttagaatgcacttgaaattcttcttcctcttcatggtgtctttgtggccagrtgtcttggaaggcccaggaatgaaggcgcctctggataaagtggccatcctgcttctgagagcatgggggacccaggcaagttactatctttctgtgagtctctgtttcctccagatgcagtgagcagcggtatagcatagggttagccagagggctctagagacatctggttcccgcaactcaccagctctgcaaccttgttaggccatgcaactcctatgtgccttattccctatttataaagtagggatgataacttccacctcacattgttgatgaaaggataaaattagataat

SEQ ID NO: 12 - DNA, 599 bp, Homo sapiens
tggtgtgattccgatgcagcccatctttcctgcctccagactgcacctcatactttctcttcatcttggataccctccttccagggttttgcttcatcagatctttcttattctccaaggaccaggtccagtatcacccttcaggaagatggggcactcttcctgaccccagtgccccatagcccatctctggcccttcctgtggtcctgatgaccatactttgctggtgctggggtctgtgaccatgcagtcctgggaggaagccaccttcaactcactctgcttctgccagtgctccrtgcaacatcttgttcatggtaggggctcacgtttgtgtggaatgatgagaatgggtggtgggggcctggctgccacagggaggtatgtgtagtctaccaaagcagcatgctccttcctggtcatgaggtggtgaccatctcatggcagccttgaagttgaaggaaagtgactactgaggctgctctccattttctcatccagaactggggctgggaacttagaagcaatggagggagcattgctttgctctgaatggaggacagtttatggctttcacgttccaggccctaggaggaaa

SEQ ID NO: 13 - DNA, 599 bp, Homo sapiens
actgcatggtcacagaccccagcaccagcaaagtatggtcatcaggaccacaggaagggccagagatgggctatggggcactggggtcaggaagagtgccccatcttcctgaagggtgatactggacctggtccttggagaataagaaagatctgatgaagcaaaaccctggaaggagggtatccaagatgaagagaaagtatgaggtgcagtctggaggcaggaaagatgggctgcatcggaatcacaccaggtagaatggttggttggttgaattgggaggtggagctggagaggtawgtagggcagatggcaaaaggccttaaattcaggccaaggagagtggacttgattatggaggcaacagagactgagggacaagagctgggattggaaagaggccccaagcatatatgaagctcccctactcatgcctgccccacaccttttcatgggatgttgtacagcacactcccagatttggaaagccccatctataaatctttcagtcccaggatcactcctttctacagtggcttcagcagcaatgacagagtcaagagaggtcaccctttaggtttctcaacacagttgccctt

SEQ ID NO: 14 - DNA, 599 bp, Homo sapiens
aggctttgtgttagatgattttgcctgactgtgggctaatggaagtggtttgagcacatttgaggtaggctagagtaagctatgttgttcagtaggttaggtgtattaaatgcattttcaacttatgatattttcagttttcaatgggtttattgggacataaccacatcataagccaagaagcatctgtattttactagggagcaggagtgaggaacaaggagagcgaagcaggcaagagggaggatacaaggatatatgatggagacggccactgctaaatgcacctcattattcaayctcatggaaatgtcccccgagaaaccatgcaatctgtgtctcaggtagggtgaccaaccatcctggttttctcaagatggggggatttttccaggatgtggaactttcagtgccaaacccaggaaggtcctgggaaaacgagaatgagcttgttccgtagaatcaggaccactggatttgaggatagaaaaatggaaagatagggaaggtaacagttgatggtcttgattgctttatgaagtgcgacatacagagatgggtgtgggaaactaaaaatggggtatgaatagttgaaagaa

SEQ ID NO: 15 - DNA, 599 bp, Homo sapiens
ctttcttcttggagcactgcaaaatcatttaagggttataaacagaagagctggtgcattagccagggttctccagagagacagaacccttaagatatatataaatgtagagaaaaagattcatcatgaggaattggcttatgtgattatctgctatctgcaagctgaaggcccaggaaagccagtggtgtagttcttatccaagcccaaaggcctgagaatcaggagtgctgatgtccgagaataaaagaagatggatgttccagctccagagaagagagaatgtacccttcctcgacmttttttgttcttttccagtcctcaatggactgggtgatgcccatgcatgttggggagggtggagcttctttactgcctgctgattcaaatgctaatgtcttctggaaacacgtgcacagacatactcagaaataatgttttgccagctatctgggtatgccttagcccagtgaagctggcacacaaaattaaccatcacagtacgcatgatttgatttatacttttaaatacacctttaaatttgatttccaccctggctgctgagtggacaatagaaagtacaggaacaagacaaata

SEQ ID NO: 16 - DNA, 599 bp, Homo sapiens
agtagaggtataaaatataaggttggatatatatgagtgtgagctagacagagctgtatgtagttttgggaggcactgcagcatattcaaagccttgggccagaggagctcaggagctcaccagggagaagatggatctaaagaaggagatctatggccaagacttgcagcactatgacatttagaaatcaagcagcaaatgtgcagggctgctaaggagactacaaagggacaactaaaaagatatgatattctagaagccaagagaagaaagcagttcaagaagggtgtggccaactstatcaaccatataatacgctgctgagagatcaaataaggacagagaagtgaccactggaggtcatcagcagcctcgtgaaaaacagtctcagtggggtggaagaaagagaggacccatagcacagggctgagcagagaatcaaaggtgaggaagtagacacaggagacaactccctgaaaagggcttctgtgagggacagcagagaactagatcagctacaggaaaaggtagggtaaggaaaaggctctgcaaaccctggagatactggaacaggtctgcattttgggggaatgatctg

SEQ ID NO: 17 - DNA, 599 bp, Homo sapiens
attaaaggagtggagagttgaaggaagagggagaagggatgggttcagaagtgaccacagaaggcaaggaggaccacttacacaacttccccacctcaggccctcaggtacccagtttttaagaggaaagacaccttccacctcagtaggttgtaaaagaaataatgtcatgagcagatattcagctttcatttaatgcaaaaaagtaagtgagcactcagagaagtaattgaggatattggaaagagtgtaattgacagccatgagttcacaaacccacagagaaaaggtttgggagcrcaggaaggaaaggaaaattgagtcaaaacagagactcttccaagtggcatgggaattagggtccccttgacctgaggagcttgggtttctggagatgactgaagtggacaggaatgaaaggcatcctgaaattagtcttaagagtctcttaggggaaggtggacttgagacaaaaggcacaatgctggttagcaacaattctctactgtgcctgtgagtaatgcagaagccagaatgagtccctgaagggattgttctcttaaggggagttcactgtgaaggcttccggggagggtcca

SEQ ID NO: 18 - DNA, 599 bp, Homo sapiens
tggagctctgcactccttgtctgcacttttggctgtgtgaccttgggccagtctctctccatctctgggcctaggtccacacatctgtgcatagacagagcaggcccaggtgccctcagagggacctcaacttcatgaggatcagaagcaccttcctgtgtggttcctcccctttgaggcctccctcgcttctctgctgctcctcccccaatcttgaagcaatttgtagtgtctgttgggttttcactgatcccaactctgcctagagcctgggaccaatccctctgtcagttcttctcyccttcaacctcctgcttgtaaattggggctaataatacctgataactcactttgctgctacagggattaactaattagtggttattaagggatagaggaatgcagtactctgctcccattaagatgactttcctcctccaacttcctgctactgtgtgcgcaatctcagtgttagtcagaaaaccctgaaagtccggacttccaggacctccatgatacagataaagaaactgaggctgaggagcaggaaggagtagcctgtgaccatgcagcgtcttgtagcagagaggggaccagaa

SEQ ID NO: 19 - DNA, 599 bp, Homo sapiens
actttctcaccttccacactcataaggtgggattaggatccctgctgtccttccctgaaggggggctttgaagagccattgagatgatagaagtgaaaccactttgtcaattagggagataagaaatggagtgtgaaaagatccaacctgggttcgaaccccagcctcactgctcatttcctgggtaaccatgataaattaatctctctggactttcatttccttaccaacaaaaggaggtgataatatgtatcaggaggatttaattagatatcagatatgaggctcttagcacactcygtgacataggaagctctactaaatggcagctcttaataacagactgtgaagtccaggagcttactaactacagtatttttttttaaccaaaggacaagaagacatagaatctaagccagactgatgtcagtgaatgtgatttacatggaaataagaaaattctgaataaacaaaactcgccataccaggtgtgaaaaataagatcctgaggcactggtatttttagcagccattttgcctgtcattcttgtttcattttcctaactctctgtggggatgagtttttaaataatttaact

SEQ ID NO: 20 - DNA, 599 bp, Homo sapiens
attattaagaggtatagcctttaggaggtgattgagtcatgaggactccaccctcatgaatgggattagatacccttataaaagtgcttggcagagggaatttgcgcctttttacacatccgtccattctgccatgtgaggacacagcattcctcccctctggaggatgcagtaacaaagcaccatgttagaagcagagcgagcagtcctcatcagataccaagcttgccttgatcatggacttcccagccttccgaaccgtgagaaatgaaaaaacccagtctgtggtattttttgtcrcagcacaaatggattaagacaattgccaaagtgtggaagtaactaagatgtccttcaatagctgagtggataaataattacatccatacagtggaatattattcagtgataaaaagaaacaagccatcaagccatgaaaacacatggaggatccttaaatccatatcactaggtgaaagaaaccaatctgaaaaggctacctactgtgtgattccaattatatgatattctggaaaaggcaaaactatggagacagtaaaaagaccagtgattgccgggggaactaaggtatgaataag

SEQ ID NO: 21 - DNA, 599 bp, Homo sapiens
ccaaagcaggtactgagtaaatatctgctaaatggacaaatacctactaaatgaatgaaagaaaaaatgaatggagggcatttgttaatctccttgaatgagaatttgtttgaagattagcagttaaattatttaaaaactcatccccacagagagttaggaaaatgaaacaagaatgacaggcaaaatggctgctaaaaataccagtgcctcaggatcttatttttcacacctggtatggcgagttttgtttattcagaattttcttatttccatgtaaatcacattcactgacatcartctggcttagattctatgtcttcttgtcctttggttaaaaaaaaatactgtagttagtaagctcctggacttcacagtctgttattaagagctgccatttagtagagcttcctatgtcacagagtgtgctaagagcctcatatctgatatctaattaaatcctcctgatacatattatcacctccttttgttggtaaggaaatgaaagtccagagagattaatttatcatggttacccaggaaatgagcagtgaggctggggttcgaacccaggttggatcttttcacactccatttct

SEQ ID NO: 22 - DNA, 599 bp, Homo sapiens
gccaaaatcagatctcatggctttcctcaatcagtgagttgagatagagacactggcaagtgaaatgggattttccctagaccaatcaagcactcctctgaagttgggggtggggtcagagacccccaggggtagctggctgtgtctgtaagtgtatgatggggttacctcaatcacactcgggttctgttaggaaggaacaaaaggaaaacggcactggcaggtagcccacgatgtgcactgcactacgtccaatgattaccttggccaaagctttccttaaacttgggccacatcaarttctcaggcaggaagttttctccccctagtctctgcaagagaatggtgatcataagacatccacacacaatgtccagaatcaaccttcaggtgtcgacatttgtcctagtcactttgagaaaggttctctggactgctgtcatgggatggactaaagtgaggtggtgctgaatgcaggaagtcagtccagctgtcagcccagcaccagtaaaatcagcagacaccccagcccaggaatgccaaggcatgagtcaggactaagttttcggtcagaagggtgaagaaaagaagggtgtgtt

SEQ ID NO: 23 - DNA, 599 bp, Homo sapiens
tggtgacgttattgactagtttgaaaacaattattttcgtggtaggataaggagacagccaatggtgttacaatgtgttactgtttgatcgccattatcgtttccaccaaatttgtggtctgtatatgcacgcttgtggaatgcattttggagtactcaaaacaacacagaagcagggctcaaaagattggaaaatttaataggaaatgctaatgtcagtgtgtattgtatcatagaaggatttcaaaaagagcagtgccacatagaaaatgaatgtgaatgtattttccaaggagatcygtgtccaaaaaaataataagcagctatttatcatgatgcaagacttcaaaacatagttaataatcatgaaaatcagctagctcttatggactatctctgtgcaattgcccataatctatccctataatacacttttcaatatgtcaaattttctttttagtttttaaatcttttggggttttccccctactatttaaaattgtcagtattattttttacaattcactacccttcatatctcatctttgcatcatttccaatacttgtagtataaattgtatagaggctttcagagttct

SEQ ID NO: 24 - DNA, 599 bp, Homo sapiens
actacagatttaaaatattaatgtcctatgacctagcaattccacttctgagaatctattttttaaataatgaaagttgcaaagactcctgtacaaacatattcattgccacattatttataataataaaaattagaaacaaatgtctgttttaagctgggtttcccccagaagttgaccctgagacaaggttatggatgcaagtagtttattttagaagtgaccctaggaagcaccagtagggggataaggcagtgggtcaggcaaggaaagaaaccctgtaaagggtaagttatcaarccggtttccaccatgggcaactggggctcagtcccacaggggaaccctcagagacactgcaaatcatacctcagaatgaccccacctgacaagtgaggaagctggagtatttacccatcaactctatatctgtcatcattttctgtgactcctgggggcattaacttcctagaactttcattttgttccacatgcaggctaagtatactctcatagatagaagaaagtcctcaggaaagtcataggtgttcccagtaagcagcctccagtgaatagaggtaagtcctatgtgacacagc

SEQ ID NO: 25 - DNA, 599 bp, Homo sapiens
  variant (300)..(300): a,c,g or t (any base)
atgttcatgaattgaaaggctcaatgtcatacaaagtattagcccaagccactaatttatgagaaatacagaagataaaaacatgttgaactacaccataaaaatacaattcagaatatggaaaactctataagaccaacaatccagcttcttcaacaaataaattccagagaaaataaaagatggagcaatactttatgtattaagtgagataagactactgaaagccaacccaaaacaaaatgtagaaacaaaaacaaataaattcatgaaatgaatgaatgagttggaaatttaaancttgacttttaacaagtcatcaagttgcccttgatgatattaaagacttattgttaatttttggggagtacaacaacagtattggggttttgatatcttttagagatatacactgaaatatttacagatgaatttaaatgacgtctgggatttgtttcaaaataatatgagcacaggggacgatgggggcacagatggtaccaaattggacaggattgatggctgttagggctgggtaatgagtacaaagggatacatcatactatttgttttgtatatgcttaaaattccaatttttt

SEQ ID NO: 26 - DNA, 599 bp, Homo sapiens
ttaacctctctggaccttagcttccttatctggaaggtggggataataaaagcacccaacccacagaattgttttaaggattaaatacattaagccatgtgaaacacttagaacagggttggcacatagcaagtgttcaaaatacagtacctattattattataggcatttacattttagtaaagcaataagtgtgtctccaagtcaatgaattccatctcggtgtgtgaaagttgtagctgcaatatcgaaagtactccctgtaatctttgagaacaactcgcagagaaaaggaaaagyagcaaaaagttgagtagtgttaaatgttttgatttccaaaataataaaagaaaatgtatctgaaagactgcacttatcctcttatcaacaaggcagtctgtccgaaaatagaaaacaaatggcaaagagatgaaaatagtggagaaaatgtaagaaaaataaggacgaattcagtaaatttgactaatagtacttccagacagtgaaaaaaagaaggtgaaaaattattttctttaaaaagagattgatttctccagaacccaagggcataagtctccagaatgaaagaatccttgaga

SEQ ID NO: 27 - DNA, 599 bp, Homo sapiens
ctttgatacctgagaagtcttagtgcccccgaaaatcccctacggaggtagtgactatgaactagaaaacagtgggagcatagttgaagccaggcagtctgggtccagagcttcttagtaggtgcatgccactgtgcccagctctatgtttaatcttttgagaaaccaccaaactttcccatagttactgcaccattttgcattcccaccagcaatgcatgaggattccagtttctccacattcttaccagcacttgttattttctggggttttgtttgtttgtttctgttttcttatartcatcctggtaagtgtgcagaggtatctcattgtggttatgatttgcatttccctagtgactaataatgttgagcattttttcatgtgcttaggggccatttgtatattttctttggaaaaatgtctattcgagtcctttgcccatttttgaattaagttgttttattgttactgagttttagatgttctttatgtattttgaatattaattccttatcaggtatatgctttacaaatattttctctcatttactaggtcatctttttcactttttgatagtattctttgatgcacaga

SEQ ID NO: 28 - DNA, 599 bp, Homo sapiens
atcagatttgcaagtgtggacgcctggagttttgttgttcagtaaaaagggcagcccaggccaaagaattaagtacaagtaaggctctgagaactcaagaaaagacagagaggctttggggccaaagagtaaggcccaaagacctgagtttccttctcagcccaaggcttttctctctacaagtcccttccacaatccaggcccagtctccttccagcttgaatttccatggtttctttaatagaagaactggttacctctgcgtctgcatgcaggggaatctccgtcaaggaatctggwggaattttccttgactccttggacccctctcgtttttatttacaaagtcgcccattcaaaactgaggaaagcagctgttgcagaagaacatcttctaaagattcccaatagcacctccagccccctatgcacataaaatggcctgctgcaggctggcttctccaggggtgctgggaatacttgaagggctgaagatcagtgggaagcagaaatcactggcctgcccacatcaacaccaacccacccctgctggattgggagaatgatttccaagggtctggaacctgcccttcctaact

SEQ ID NO: 29 - DNA, 599 bp, Homo sapiens
tgtgtggaaacgctcaaagcgtttgcttgacaaccttctgcggcagagggttggtcgaacgaagggagaggggctttacactcgctaaacaaaactcctggtttccttcttcccaagtaatggagaagcaaaaaacccagatttcgtcctgtttcgtttttacaaaggaggaaaaacttcaaattttaaaatttttaactggattatattataggcaccttttaatacgggatttcgtggctctcagtaactttcaagattctgagattcagatatctagattttaaattctggacgacsgttgtcctaacgtgattccataaatctaaagaatgcgatcctgcgatcctgcgaccctgtaattctaagattccaagactgggagtctaactttgactatgcttatattcctttgacttaagcttctagtcaaagtctaaaaacctatgtattaagatactgggactaaaccaggcacggtggcgggagcctgtagtcccagctactggaggttaaggcaggaagatcgcttgagcccaggagttccaggctgtagtgcacaatcattgtgcctgtgagtagccacagcactccagcct

SEQ ID NO: 30 - DNA, 599 bp, Homo sapiens
gtcgcccggccagttcggagcgctgggagcctgctacaaccctggcgggcagctcggaggggccagtgcaggcgcctaccatgctcgccatgctgccgcttatcccggtgggatagatcggttcgtgtccgccatgtgagccagcgtagggacgaaaactcatagacacatcggctgttcacacgttccccgcaatctgagaacgaacaggaatggagagaggactcaactgggacccacgtggaaaagaccgagcaggccacagaggctcggtctccccgcgcacagcgtaggcaccckgtgtactctgtaaacgggaggaggtggggcgaggcagccagagcccttggactggcacagggaccctcgatggagcgaagccctcaaacgggatgctttctggtattctatcggggagggtccttggcggtaaccagagggcagcgtagtgtcaacaccagagaccaggatccaaattgtggggaatcagtttcagccttccatgtgctgccggaactcgggcctttttacgcggttcgtcctctagtgcctttaactgcgttactacaataaaaggctgcggcagcgcctttcttctt

SEQ ID NO: 31 - DNA, 599 bp, Homo sapiens
ttgtttaatagctctagtaaccttgtggattctttgggattttctacatataagatcatgtcatctgtgaatagagatagttttacctcttcctttccagtttgggtgccttttatttctttttcttttcggatagctctggctagaatttccagtacaatgttggacagcataaagcaaacattcttctcttgttcctgttattaggaagaaagctttcagtttttcaccactgagtatgatgttaactgtgaacttttaaataaatacgccttatcattttgaggaattttctctctrttcctagtttcctgagagtttttcatcatgaaaggatgttggattttgtcaaataatgcaacatccttttctgcattgagatgatcatgcagtttcttcccctcttcattgtataaaagtgatggattgattttcttatgttgaaccatccttgcattcctgagataaatctcacttgatcatgttgaaaatccttttaatttgctctaggattttatctattaatattttgttgagaatttttgcatctatattcaaaaggaatatttgtctgtaattttcttataatgtctatctag

SEQ ID NO: 32 - DNA, 599 bp, Homo sapiens
tgtgaatagagatagttttacctcttcctttccagtttgggtgccttttatttctttttcttttcggatagctctggctagaatttccagtacaatgttggacagcataaagcaaacattcttctcttgttcctgttattaggaagaaagctttcagtttttcaccactgagtatgatgttaactgtgaacttttaaataaatacgccttatcattttgaggaattttctctctgttcctagtttcctgagagtttttcatcatgaaaggatgttggattttgtcaaataatgcaacatycttttctgcattgagatgatcatgcagtttcttcccctcttcattgtataaaagtgatggattgattttcttatgttgaaccatccttgcattcctgagataaatctcacttgatcatgttgaaaatccttttaatttgctctaggattttatctattaatattttgttgagaatttttgcatctatattcaaaaggaatatttgtctgtaattttcttataatgtctatctagctttggtatcagagaaatattgccctcatggaatgaattaggaagtgttccctcttcttcaattt

SEQ ID NO: 33 - DNA, 599 bp, Homo sapiens
caccctacaacctggactgaggttttttcattattacactcatgacttctgctccaatatgtgtttgaacactgtttgagtgagaggcggtgtttcttaacattctcttcttctgcatatccagaacaaataatacaaagggaatctagaaactacctaaccccatagccagatcattctgacaatgctatagaaaaattaggtaggatatgccatattataaagtagggccatgagttcaaaaaacaaataacacatggactagctagggaagccacaataggtaacagcatgcgtttwaaaggcttagccaatgctaagctccctgtgagtcaggaatgtcatgaggcttccataataataaacttcatttgtagcagcattaaaagaaatagagtgcccagagtgaggaggcaatggtcatacctgactttgccctgctcagggtgtctgaattgcccatgagcttcacactttttgatgggcattgattaactggactatgtcatagaagacaaacggaatggcgcagtggctgcaacctgtgtcccaggaggaggaattaggtgaattaggcttgtgtagcctgaagaaagcca

SEQ ID NO: 34 - DNA, 599 bp, Homo sapiens
tttcctctcttagtttcatcgtttgtctaatagcaaagagcgggaaggctggggtgggtgtgctgacttgagaatccactgtagatgagagtttccctcacctgcccctccttaactacattggaggtgtgtcattcgctgccattgaaatggtttgggtggactagctggccttgatgatagggtgcggtggaggggacatgcaggggttgaaagcagacacctgagatggaagcttgtggtaccttatgtgatttgaggcagggccagctagccgtccaccaggatgggtgcatctgrtgccacagggtgagtcatcactgcacaacaattgctcagccagcaactccatttcccaggcccctttgcactaagtcaggtacagtacagtgtttcctgttgttgacagctggcatttgttccgattggctgggtgtccactccgactggttggtgcgtgtgctgaactagccaaaaagtattttttctttttttggttttaaggagcaaaaagtttaataggcaagaaagaaggaagaaaaaagagaccagctccctcatacaaagacaagggaggaggacttggaacaaagagaaac

SEQ ID NO: 35 - DNA, 599 bp, Homo sapiens
ctgtctgaagacgccacctcctctgtgaggcctctcctgatttctgagaccacacaaatctctccctgggctgcgttttcatggcatgctgccttcagtcaagggaacacatgcaagcagaaagagctcagagctgtgtgagaaagaaaagcagcatgtgtggtctcagaaatcagaagaggcctcacggaggaggtggcatcttcagacagagccaggtggacagagggattcaggctgaaggagtagcacagagatcagcaaggagaatggtctagtgcgtggcacacatagtactgrtgggaagggtgtggcctggcaggcagggtcacatcatggagatctatgagtgttctgttagggtctggaagtttctcctcagggcatgaggaaccatggaagagattttgttttttcagtgtgtatgcataggtggatgtgttttagtaaactttttattgaaatgtaacatatgtatagaaaagtacaaaatttttcacaaactgaccgcatctacgtaatagcactcagattaaaacaataacaaattattactaaaactccagagatccactctggcctctttcaggaactactta

SEQ ID NO: 36 - DNA, 599 bp, Homo sapiens
aggtagaaatgaacttaagagggagatggaggaaggtttctgtatggggtggctggctggatgagggacttcagatttgaagccaggagaaaacatcccaattttataacatcatgtttgaaatgtaggacaatgagatgaagatgtccaataaactgttagctatatgagtctgttgcttagggatgaggtcaaggctgtagacagagttaggagttatcacagagatgggggttgaggctggggggtggctgagatttctcaggtggttgtgtgcaatgaaagagacaagggctgaaraccagacgcctggcttaggggatgagcggaggaagaacaagagacaacatcgcttccctctgaaatgctggcacgggctgcgaagcatgagtcagacccttctagccccagcagcagcctcaacagcctgggtctatctcaccagccacacagttttctcctgaatgaacttgcatcactaacctcagaagctgtgttgcgaaatccgcatcagattgcgtacctctggtggcgtcctagggacaatgggcactaaagactcatagcatcctggggttggagtcctcctctccgcctcc

SEQ ID NO: 37 - DNA, 599 bp, Homo sapiens
taggagttatcacagagatgggggttgaggctggggggtggctgagatttctcaggtggttgtgtgcaatgaaagagacaagggctgaagaccagacgcctggcttaggggatgagcggaggaagaacaagagacaacatcgcttccctctgaaatgctggcacgggctgcgaagcatgagtcagacccttctagccccagcagcagcctcaacagcctgggtctatctcaccagccacacagttttctcctgaatgaacttgcatcactaacctcagaagctgtgttgcgaaatccgcmtcagattgcgtacctctggtggcgtcctagggacaatgggcactaaagactcatagcatcctggggttggagtcctcctctccgcctccacaacatccctgccaatcagttatcgagcaccccaggcagggaactcataacttcccacggcatcccatttccttgttgagcaagtgaagatctaagaaaaccttttcttgcaatctcacgatactgaaaagtcaaagaaaaagataacgctataatttaatggtaatgatccaggacagagtgaaatgcaaggactctgcatcctggaa

SEQ ID NO: 38 - DNA, 599 bp, Homo sapiens
acacaccatatgctaaaactaactcaaaatggaataaagaccttaatgtttatagacaatgttataaaacttataaaagaaaacacagatgtatatctttgtgatgttggattactcaacagtttcttagatataataccaaaaacaaagcacagtaacaaaagaaaaaacagataactggactttatcaaaactaaaaactttgcatgtgaaaggacactgtcaagaaagtagaaagacaaagaatgagaggaaatatgtgcaaatcatgtatctgataaaggtctaatagccagaatrtacatatttttaaactctcacaactcaacaataaaaaaatttgaaaattagtaaagtacttcaatagacatttgtccaaagaagatatacaaatggccaataagaaatgaaaagatgttcattattagtctttaggaaactagaagtcaaaaccacaatgagaatttggaatctttgtgcattgctgtagtaatgtaaaatggtacagccactgtggaaaacagtttggcaatttctcaaaaagctcaacataaaactagcaaatgacccagcaattctattcctaggtatacacccaa

SEQ ID NO: 39 - DNA, 599 bp, Homo sapiens
gcggcccctctggcccgggttcgttccctggccattcactgggagatccagggcctgtccttgtcccctcgcagggcctcagtgttctactctgttaaatagcagctggagggcgtgtgctgatctccgaggcccctgctacgtataggattccgggccttggcgcgggttccagtggctccagcccggacgcagctttccggggtggggggggggggcgggccggggctgtgggtgcagagccgcgcggatggtggtgccaggtgagtgtgggagatcccaagacgagagttctggccrgtaacgggtggcatgccttccagggttttagtcgcgttcttcgtggcggtgagctgagggcaggctaagcccggccaagcgttgggcagctgccttcggaagagcgaggcaaagcccagatccttgctccgtcgacccggtgtgtggaaacgctcaaagcgtttgcttgacaaccttctgcggcagagggttggtcgaacgaagggagaggggctttacactcgctaaacaaaactcctggtttccttcttcccaagtaatggagaagcaaaaaacccagatttcgtcctgtttcgttt

SEQ ID NO: 40 - DNA, 599 bp, Homo sapiens
gcccgggcgcacctccccaggtaggagcagctgtggcggcgcggtaggagccccacagcgtcagggcggggagggcgcttggagatcccccgggtcggccccctccgcaccccgatcgactgaggcccggagagttcaggaaccggctcgggagacctcagcgcccgaactcggagttttcggacttttcaggcctctgccaagggcttggactcgcaggtgaaaggacgggctcatctcacttttttgtcctgagagggtgagaattggccgagagggcgcttggtttttgtgggaatrgggagggagaagttccaggagactggctccccgggcctttcaggatggtttccaaaccccaatcgtggctgccacagccactcggcactccagtccccccatctttcgaatgagtattcctaggcccgaaagggaaggtgacttgccatggccccactgcggcttctccgcttagcggagtctccaacctgcgtctcctttaggactgcgcttcccaactgtgtggtagaggtagccgctttccctttttgggccccaggtccctcgactgtaccggggtctggatctctcaagagtcc

SEQ ID NO: 41 - DNA, 599 bp, Homo sapiens
ccggtgggatagatcggttcgtgtccgccatgtgagccagcgtagggacgaaaactcatagacacatcggctgttcacacgttccccgcaatctgagaacgaacaggaatggagagaggactcaactgggacccacgtggaaaagaccgagcaggccacagaggctcggtctccccgcgcacagcgtaggcacccggtgtactctgtaaacgggaggaggtggggcgaggcagccagagcccttggactggcacagggaccctcgatggagcgaagccctcaaacgggatgctttctggyattctatcggggagggtccttggcggtaaccagagggcagcgtagtgtcaacaccagagaccaggatccaaattgtggggaatcagtttcagccttccatgtgctgccggaactcgggcctttttacgcggttcgtcctctagtgcctttaactgcgttactacaataaaaggctgcggcagcgcctttcttcttaaagtgaggaggacaaatttgcaaaagaaataggcttttcttcttttttaaattggagaaatctctgctctggttgacctgggctggttttccctgtctctgag

SEQ ID NO: 42 - DNA, 599 bp, Homo sapiens
ttctttccatagctgggcccctgctttgcttaaaagagtcttgtagggcaaagcccgagtggtcagagagctactggaacaccaaccccacaaggcagctaactgacagcctttcctaagccttgtcaactccacctcctggccatttcagaagcaatctttcctctccttcctttcacttctttccatcctcgcaacccttacacacatccagggcttcaccactgtgttagtcctctgacccagagctctgttccagcagtatgtttggtcccaccacctatggacagtttggcctcrccagggctcagggaaggcagggagcgctacacaccagccacccaaggtgaggccccctacacagacctgagcagctgttcctcatcttttcatattcctgaattacatttaaacccacaacaggagggcaagctcagctctcctggccagccccagggttccctagagtcctaccctagtggctgatagacatgatgaccatgcggatggcttcaggactgacaaaaacacccctgaacctatagaagctgggaccctgctcagtgctgaatctttccccgaccctcctgccaaaagtt

SEQ ID NO: 43 - DNA, 599 bp, Homo sapiens
cgccagggctcagggaaggcagggagcgctacacaccagccacccaaggtgaggccccctacacagacctgagcagctgttcctcatcttttcatattcctgaattacatttaaacccacaacaggagggcaagctcagctctcctggccagccccagggttccctagagtcctaccctagtggctgatagacatgatgaccatgcggatggcttcaggactgacaaaaacacccctgaacctatagaagctgggaccctgctcagtgctgaatctttccccgaccctcctgccaaaagytgggctacatctgcccaggcaaaggccagggctacatgcacatgcaagaccctaagtttccaggacaccagggagaaaggttgctcctgtgagaccctcagaccccagcccagtagggccaagttagccaaactctccctcaaagcttcttatttctagaccccaggataatccctgggtccccagtacctgaacctagggtagaagatggtgctccgagtctgaatctcccctcatgcctgcttatcacctgcaagacccctgcttctgggctgctttgcagttgcccacctgtctag

SEQ ID NO: 44 - DNA, 599 bp, Homo sapiens
tggcgcttacggccaccttggcctcgggggcagggcatgggcggcccccgccagatcgcccagcgccagtactaactgccctcgctctggccttcgagcccgaagcctcttctgcgcgcacaacctaggcagtaatcctaaactagcgggcaccacagaccagctgcagccaccccaacccagggatcacttccggacccctcgaccgcccggcaccagcgcgcaagggacccttcagccggagaccagagtccagtcccggtcacgaggccaccgccgctgcccgcctcgagaagcacmacgcgggctgagccgtcggctagcgggtcactcccgagcctctgtctgcaccgcgccagccccagaccacggacgctgagcctccagcgcgtgccagcctgggccgctgggctctcggggccagcccgcgacgatcccctgagctctccgcagaagggccgagcgtccgttccggggacgccaggcccgcccccgccccccgacagccgcggggatccagagcccgggggtgcgggacgcccgcgccatgactgccgagagcgggccgccgccgccgcagccggaggtgctggctaccg

SEQ ID NO: 45 - DNA, 599 bp, Homo sapiens
ctgggaccctgctcagtgctgaatctttccccgaccctcctgccaaaagttgggctacatctgcccaggcaaaggccagggctacatgcacatgcaagaccctaagtttccaggacaccagggagaaaggttgctcctgtgagaccctcagaccccagcccagtagggccaagttagccaaactctccctcaaagcttcttatttctagaccccaggataatccctgggtccccagtacctgaacctagggtagaagatggtgctccgagtctgaatctcccctcatgcctgcttatcayctgcaagacccctgcttctgggctgctttgcagttgcccacctgtctagatggcctacctttccattaccatgccttccagatccagtgctctgccagttatcctggatatgctcccaatgcccagttagtgacaggcctgagagtagcccctgggtctcctgatacattagtgtatttaatatgtgcaaagtggttacaacagtgtgtgttcatggaagagttatgtatgtctgagttaatattactaactttcacaactctcttacattactatttatccccattgtatagatcgac

SEQ ID NO: 46 - DNA, 599 bp, Homo sapiens
gaggctcgtatgggcaactggaacccggctggagtgtgcagctggccccctcacttccagcccgcaggctgccgcgggggtgactcccgggagatgcgcctagagaggcaggacacctgcacgcatattcgtaacagaggcagaaatactgacggcgaaacctagagagacttgcactccaggcccggggggtgggggggcgggggagaagccttagggagagtggggaggaggccagaggagagctggctgctctctcgtgttgaggttcaagtgtcttccagctccccgcgccccctsctcaccgtgccaccgcccacctcatgctttgggcgccaagcagccaagcaaccgggttgcgttgtagggggaggcaatcagccctgttacccctcctcaggcggccggactgagctgcgggatgggatccctacagagactggcctgggtaaggggaaaaaatcactgcctcttcctgtgcctctagtggttgaactagaatcctggctagctcatctcgcttttgaagggggaaggggagtaatcatcggctcttttgagattgtcttgcaaaccatggactctctccccagaaatga

SEQ ID NO: 47 - DNA, 599 bp, Homo sapiens
catattaccttcattaagatgataatcaaatgcagtctccattgggggaggcagaggttctagtccagcttggacacttgtaactgtcatatcttgggcaagtcacacaccccttctatggctctatggaggctccacacacactcacaatattcctatttatttctattgggaacttatacaagcaatgagatgaaaatcctggttctccccatgtggtgcaatgtgatcctgggaacatgctttcccaatctgggccccttccccatccttcatggtacatgcccctgtctgaactgyttttcatcagggacatattcccccagctatcttgtcatgtctaccctcaggaacaggctgagcacacacagaaagtcaagctcgtatgtgttcaaggcaggggagggttccttagggcactgaggaggaccttaggcttttactagaggatcctttggcccagactgacttggagacagggaccccttccagttggccattgtgtcctgaacagaaagcttaatttttctcctaaaccctcaactacctcagacttggggctagagctgaggggtcagaggcaaagtttattctatact

SEQ ID NO: 48 - DNA, 599 bp, Homo sapiens
acatggtctggctgggctttgagctgggtcacacattggctgctgtagggaaaactgccaagggagcaggggaggaagcagggggtcctgttaggggctggcactgtgatccgggagagatgacagggtcctggagggctggcggctgtggaagcgtggcatggggttaggtggttcctggagagggaatggtcagggcaaattcatgatgacaggaacacatgggagtgtcagggaacagcaaatggtgtgttggctgcagcctaggggtgtgggaaggcagctatgggagacgtggcyggcaagaaggttgacataaggatgaccgagggtcccaagtgccaggtgaggcactgaagcgttctactgcagacggcaggcagccttctccacaggactggagctcagcttcagggaggcaagctggcagcacggacagaatggaggcaggagtactcttgggaggccctggctggctgctggggccgtgagcagaggtgggagttgagtcctgggaggtggtggctctcaatttattgcctggttgcagagcacctccatgcctgagaaaccatgactggggctctgccctgaccacc

SEQ ID NO: 49 - DNA, 599 bp, Homo sapiens
ttccctttgagacttctcttggcttcttataaataaatgtgcagctaacagactatctagccctaccccttgttttatggaagagaagccagagaaagtaactttccaaggtaacagtgagttatcattgtagaccctagttcagggcccttccacagctccaggggctcagcaggaacaaaactaataataatcccaacattttccaaaagccactttgttcagggagtaccagccattttcacaggtgttatctctttggatcctcacagctcccctgggaagcagaaggtttcaaaygcacccttttcaggcaaggaaactaaggtccaagaaaaggggtaatgagctcaagtcatgtgaccgttgcacacaggagggaccaaccctcaacccgggtgtaacaaatcctcagtaaaatgaaccagaaatctgaggctccttcctccctggatgtaggaatcaggaggctgggagaatcacctgttagtgttgactggcgcttgggaatcctctggcttcgagcctctgagtagggtggattctccatatgcccctggcttatctccatacggcatgtaaggcttaaagatagtgtc

SEQ ID NO: 50 - DNA, 599 bp, Homo sapiens
aggcaccatttctgccacctcgaggaggtttaccttctgccctccatgatgtgaccctgtctagcaggccccctcctgcccacctggggcatgtgtgccatctaccagcccactccacattctctgcgcaggtctctgtgctcttccttcagcctggaattcttctgtccacctggctctgtctgaagacgccacctcctctgtgaggcctctcctgatttctgagaccacacaaatctctccctgggctgcgttttcatggcatgctgccttcagtcaagggaacacatgcaagcagaragagctcagagctgtgtgagaaagaaaagcagcatgtgtggtctcagaaatcagaagaggcctcacggaggaggtggcatcttcagacagagccaggtggacagagggattcaggctgaaggagtagcacagagatcagcaaggagaatggtctagtgcgtggcacacatagtactgatgggaagggtgtggcctggcaggcagggtcacatcatggagatctatgagtgttctgttagggtctggaagtttctcctcagggcatgaggaaccatggaagagattttgttttttcagtg

SEQ ID NO: 51 - DNA, 599 bp, Homo sapiens
tccagagatccactctggcctctttcaggaactacttacatacaagaaaaataaccatcctaacatcataaactagttttcactattttcttgtgtgtggcttttttcactccacatatgtttgcaagactcccccatgttgttagacataaatgcactttgttcttgctcattgctggttgatatcccattgtatggatgcactgcaattgttgatgagctttgtgcagtttccagtttggggctgtaaaccttcctgcacatgcctttacgtgctcctctgggggtaaatctgttgcrtgcatatctaggagtggaattgctgggttgtgggctatgcatatatctacttgagtagatacttctagtttcccaaagttgttctacccatgtacacgcccaccagggatgcacaggagttctcattgctctatattcttgtcaacacttggtattgtcagtctttttgtgttttatgcttgtttttttttttttaggggaatgaccacctggtggaggtggagtcccacttgggttgaagtatactcactctggcaggaatgaaatcagtggtaatagagctagaatgaaaaaggcca

SEQ ID NO: 52 - DNA, 599 bp, Homo sapiens
gctcctgacctcaagtggtctgactgcctcggactcccaaagtgctgcgattacaggcgtgagtcactacgtcgggccatcctctaatcatcttttcttgatttttgcctttcccaaatccaggtttcatattcaaaacaacaaaattgttaagatgttttcatatctaaacaacatttgggtgttctaaagtaaccaccaggtaacacaatggtttgctcctatactacctatgaaaggaatgacgaaaggaataattaggtttgtatagaatattgtgcattatagtcataactgctrtgagaactcccaaatcagggtggtgtgcaggttagggcagagttttgggagaggcgaactgttctgtacaaaaaggagaagaatcataatgctgaggggaggggagggacctgggagaggggagtgagcagcaagcagggtggggcaccacctgaatgaaggacagaaggtgagagcccttaggggcacagcagcacaaaagactttgggagaaaaggaattaataagagggaatttttaagttgtttgtcatggtgtgtgctatgggcctccttccctcttgtcactcttacactgtc

SEQ ID NO: 53 - DNA, 599 bp, Homo sapiens
cacccactagatggcgctgtgaggtgcagtgggaagagtagctggaggcaaacctttgagattcagattttggacaaatatttaacttcctggaggctcagattcctccttgagatacacttggcctgtatcccccacaacacaggtatgtagatcatacgggataacgtgtaagaaagcactttcaagtataacacgtggtgacatacttgagcctgtggactgttgtcactgaccactccaccctgtgtaccagaggctggggctgtcagagtctagtgtcccaggaccttccccagscttctctatgatcccttcactcctaccttcaccacgtgtgaccaagtcccctggtttcccagctaggagagattgccagaaaggagctcatgttgatactgtacttgcttgctctttggaaaagcacgtattgattaatctcttcacctttgctgtaccccttttcctagcatggcctgtgtgactccagggccctagtgctgctgaggttggcatgggaggggaatcccataggaagggaatgtgaaagaaagccccagcatgaggaataagggaagttccacaatgcccatggtgtg

SEQ ID NO: 54 - DNA, 599 bp, Homo sapiens
gaggcaaacctttgagattcagattttggacaaatatttaacttcctggaggctcagattcctccttgagatacacttggcctgtatcccccacaacacaggtatgtagatcatacgggataacgtgtaagaaagcactttcaagtataacacgtggtgacatacttgagcctgtggactgttgtcactgaccactccaccctgtgtaccagaggctggggctgtcagagtctagtgtcccaggaccttccccaggcttctctatgatcccttcactcctaccttcaccacgtgtgaccragtcccctggtttcccagctaggagagattgccagaaaggagctcatgttgatactgtacttgcttgctctttggaaaagcacgtattgattaatctcttcacctttgctgtaccccttttcctagcatggcctgtgtgactccagggccctagtgctgctgaggttggcatgggaggggaatcccataggaagggaatgtgaaagaaagccccagcatgaggaataagggaagttccacaatgcccatggtgtgggagaatgcaactcccaccaggcctgctgggccttctatctatc

SEQ ID NO: 55 - DNA, 599 bp, Homo sapiens
gctgacatgcaaaacaacacaggctcccaacccttgtgaaatactgaagacaagtcagtctggacagtcctcagcagctcccagatcccaaaacctggcagaaccatttgcaacaggaatggagccagcttttctggggtctgccctctgcacagcgagttcctggaggctacagtcctggtgtctggtagttgatccccagtagattttccatttgtcttactcactgctctcctaagggcctccagcctaatttaggccaaagaatctgcattgcatcccccgccccgtcccaatccmagccccacctcaggcccaccttcaatcctctcccccattctaactctcaccccagattagggcctgtggggtatctttccctagagcccagaaagacgtacagtctctcatcccaagggaaattcaatgactgaattggcactgattgcaaaagcaactagaatatttgtggcatttacttttttttggtccaaagatctcaggaaaccttgaatccttcagctgtcaacattcctcaggatagcgtggcctctcagaccagagcacccatctccatccttgctggagaatgtcccgtc

SEQ ID NO: 56 - DNA, 599 bp, Homo sapiens
tctttttcattcctgatgttagtaatctgtcttctctctttttttccttggccaatctggctagaagtttatcagttttattaatccttttaaataaccagcttttcatttcactaatattcctctgttgttccgttttcaatttcattgagttctgctacaataaaacatttaatttctgttttgtaatgtttctctggatgctttcaaaatacttttcatttcctctcatttccagtagattaataataatgtgtctggacattgttttctttgaatttatcatatttcagatttgtkgagtttcttctttttcttttttcttgagacagggccttgctctttcacccaggctgtagtgcagtggtgcaatcacggctcactgcagtctcaacctcccaggctcaagtaatcctttcacctcagcctcccaagtagctgagacatgccaccaggcctggctaacttttttttttttttttttaagacaccaggatctccctatgttggccagactcgtctcaaactcctgggctcaagtgatccaccacccttgttctccaaagtgctgggactacaggcgtgtgcctggccttgct

SEQ ID NO: 57 - DNA, 599 bp, Homo sapiens
gctgttaggctacaaacctgtatagcatgttactgtactgaatgctgcatgcaattgtaacacagtggcatttgtgtacctaaatatatctaaacagagaaaagaaacagtaaaactatgatatcatgatctcgagggaccactgttgtatatgcagcccatttttgaccaaaacattgttatatggtacatgactgtattatgctgtaagatgctggatcctattaaagtcctctggggaacactgatttttttttttaacctaacaattctttccagttaggtttagaccacaaattwtgactcaccttctgtggacagaggttccaaagtcagttcagttttcaaagccatcccctgtctcagcctcccatttaacaccacttgctttcccttggcccattagttttaagcattatttatgtcatcttactatatttttgaaggcattttcatatgtgaccttgtatcttggcaaagatcagatattaggcaaagaactaagaaatacaaatctccagctccagtatgatgaggaggtccctgccttgaggtccctgagcgccctggataacctgcctataccctcttggactcag

SEQ ID NO: 58 - DNA, 599 bp, Homo sapiens
gatcctgccttggttctgtcagttctctcgtatctcaactcctcccctaatcccagcagagcctaaactcccaagtctgttgacaaccctactgcacccctagcccaggctctgctccatcacctgccaacatcccagcctggcctttctcctaggtctgcccacctctgcatcagtacagccaccttatccagaggggcacagccaatcttggactccaagcctctcctttactgttgactcaagtgactgagctcagacttcccattctgacagcggcaaggtccagtggtaggaagrgcaattcctaccactcatagcctagctcactacatgcaagtagacagtgaaccagtgggtggagctgatgagtgagtgatctgcaggcagagactccagattggatgaaccacacagttttggcaagcattgggtcaaggagctctctggtggctcagccctgccagcaacatcatccacatgggcgtctgtcttctgcctggaggcacagcgccttatccataaacctgtctctggaagaacagaaaacgtcagtgcaccctcagggacaaagctgcccaggcttagccagtttgcct

SEQ ID NO: 59 - DNA, 599 bp, Homo sapiens
tcatacattgaattttagggaccccctagatccccctgaagtctgcttatctagccaggtaagccagaacagagtggtagaaaaaggctatcaatgaagagaaacacaataaaaatattacctaggtttataggtcattgaagagttcagaagacactttcccaagcattatcctggcaacaactctgtaaggtcacagagctagaggcagggtgcagtgacttgcctgaggtcacgagctagcccatggcagggcagaaagcgcttgaagtttctgaggccaagcccagtgctcttcgyatttcccatcttgcttcccaggaggttccatgcagatcctgtgggaatgggcgtttctgcccttattcccctgtgcattcttcacgagtggttggatttccatggacttcattctgccaaagactcatgtgctgaatcatatggaactgctgaccttccaataaaccagcatgatttgctaaggcgtagagccaggctgagaaggaagaggactcaggccagactggtgggttggaatcctagctgtgcagccgagcagcttgagcacgttactcaacctccacccaatgtacgaaaag

SEQ ID NO: 60 - DNA, 599 bp, Homo sapiens
agcttgaactcctggtgttttcctccaagccagtgaaatgggaataaaatctgctatcaagggctccccctacaggcaccaaaaagcaaaggctaaaaattaaggtgggtgccatttacgcctagtagagaattgtttcttctcttgaaaaagtttctaaggcagtcctgaaaccacaactttgcttgttgacacaatcccttttccaaaactctggccaaagagtcttaaaaatctaaaggaccaagaatcaagcaacctcttcctcttgatgtactcacctcccacttaataaaggamcaaatgtgattttgatacaaataggacacaaaacgaccctagagctgtaacactatttttgacagtcataattatggcacttttgagttttcttgtagctcatttccatctcatttttaggattagtgtgtatttgtgtgtgtgtgtacatgcgtgtgtgtacatgcacgcaccaaaacaacttcaattccggacataaaatcaatgacagttttaaaaccacttgatggaaggtttgagggaacttttatcatttatttattttgtgctacaaatcaggtatatttaattctctgggt

SEQ ID NO: 61 - DNA, 599 bp, Homo sapiens
aaaatctgctatcaagggctccccctacaggcaccaaaaagcaaaggctaaaaattaaggtgggtgccatttacgcctagtagagaattgtttcttctcttgaaaaagtttctaaggcagtcctgaaaccacaactttgcttgttgacacaatcccttttccaaaactctggccaaagagtcttaaaaatctaaaggaccaagaatcaagcaacctcttcctcttgatgtactcacctcccacttaataaaggaccaaatgtgattttgatacaaataggacacaaaacgaccctagagytgtaacactatttttgacagtcataattatggcacttttgagttttcttgtagctcatttccatctcatttttaggattagtgtgtatttgtgtgtgtgtgtacatgcgtgtgtgtacatgcacgcaccaaaacaacttcaattccggacataaaatcaatgacagttttaaaaccacttgatggaaggtttgagggaacttttatcatttatttattttgtgctacaaatcaggtatatttaattctctgggttcgtggaatgagcttggatggttgcctgctctcaaaggctaaaaa

SEQ ID NO: 62 - DNA, 599 bp, Homo sapiens
atcccagctactcaggaggctgaggcaggagaattgctcgaacctgggaggcagagattgcagtgagccgagatcacgccactgcactccagcctgggcgaaagagcaagactccatctcaaaacaaaaacaaaaacaaaaaaacagtaacaattttcttgaacttctgagggtgatttccttttctgatacaaagacatccaaagaaatctcagagagtctctattttagaatcagtattaaatatatatacatatatatatgtgtgtgtgactatgtcacaaagtctctgaaacacakaagtttaatctcaaaaactatctctacttgttttttatttgttagtttcctgatataggtttgatttggaatcaaatagaaaggtcaggcttttccagacagacaggaaaaccaatatgaattactgaatagatcccctcaactagaaagtctctgctggaatgaactttgtctaattttatcaagtgcaccaagaacagcttctttcagaaattatcgagacagcatcaccttttgattctttaatatcaacagatcaaaaagcatttggtgatttctctgtattttatatctctttg

SEQ ID NO: 63 - DNA, 599 bp, Homo sapiens
tatgttttatacataccatattaaaaataaaatacattaaaaatcctacaaacataggaagttttgctgtttcttagggacacagatgtgagagagtcccaaagcaaagggatgttactggtaactgcgggtctgtgatactagctttctggtgcagcctctctcgcctgtagctggtgttctgggtgtgtgtgggtttagttctctttcttgtggctttctgaggatgctgggactgtcatgcatttccttgtctctgttctgagaagaaaatgacaaggaattaaagtctacattgartatagaaaagtgacattttggatatcagaaaagtgacatcttggctgacagaaaatgaaagctcacctgtcttaggagactaaggaaaaatactaccatgatttttctgtccagtaaaacaaaaatttgggggtgtacaagttaaacctaatgataataaacacctcttgacggtgacagtctccgtaatgatcaaatagttttccaaaccctgtccatgtcaagtgatctttaaggaaccaaccagattccatattgaagtttgttgtaattaaattcaacctgcatgatagtgctca

SEQ ID NO: 64 - DNA, 599 bp, Homo sapiens
tgttttgtatatgcttaaaattccaattttttaaattaaacatatatatcaaatatacatgttatcccttactcatttatagacttcatataatcctaatcagaatcccaacagaagacaaaataaatgtaacatatatgctaatagaatactaagcaacattttacatgatgctcttaaaaaatatttaactacatagaaagtcatttatacatattattaaagcaaattatgaaggattatgtgtagaaaacttccatttatataaaaaataggccaactgaggaccagagctcagayaaggagtttttgcagaaggttaagtatcagagaggtatgtccaagtgagtgcttcttaacattcactatgctttggaatcatctggggggactttttttttttttttttttttttgaggctgagtctcgctctgtcatcaggctgatgtacactggcacaatctcagctcactgcaacctccacctcccgggtcccagttcaagcaattctcctgcctcagcctcccgagtagctggaggcgtgcaccaccatgcccatctaatttttgtatttttagtagagacggggtttcactatg

SEQ ID NO: 65 - DNA, 599 bp, Homo sapiens
gagccaatgtacccagcctcatctgggaggactttcaaaatatacagatcaccaggccctacaccaaaatattctatttcaattggcctgaaatggagcccaggcatccatagattttaaaaactcttcaggtgatttcaatgtatttccaggtgattttaatgtgcaggactgagaaccaattatctagatttacaaagagaagcatagaaagatgggtggagaatagaaatgtgatagagatgtaatagatatatgattgatggacagatgaaagatttttaataggtagatagataygtaggtgacacatggatagatgatagatttttgaaacatgatggatgagacagaaatatggatagatagatagataataggtaattaagtagataaacagtagacctgaagatctgaaggaagggaagagaaaaaagaggaaaaagggatgggaaaagaaggaatggaatttggaatgacaggtcagaaatggcccacaactcagaccctcaatggacaaaagaaaatgaaacaaaaatgaatttggaaaaataaaaggtgaataggtacagctaatgcaatgaaatgtaaaagttgtc

SEQ ID NO: 66 - DNA, 599 bp, Homo sapiens
ctgggactataagtgtgcaacactgcacttacctagttttttaaattttttgcagagataggggtcttgctatgttgagaaggctggtctccaacttctggcctcaagcaatcctctcaccttggccttccaaagtgctggggttatagaatcacacttcttgaccattctcaaaaaccaccgccaccctcaaagaaggtggccaaatctacacagatagaactttacctgtccctcgctcaggactctgaaagagcattctctgcccagagccatgagcaacctgaaagcagaattgcrccccatttgtctaccccaagtgatcatcacagaaaccaacacttagtagggactcaataaatgctattgaatgaaagaaaacataaatctgaatgaaaagttagccacaagacatgtcacatgctgtgacgaaaagagttgggaatcaaaagaactggttctggctctactaataatgcattatgggcactcactggccctctctggatctcagggatcccattaattgaactgagagaggtggagtaggtattctttggcagtccatttcagctctgattttgtgtgaggctgtaggg

SEQ ID NO: 67 - DNA, 599 bp, Homo sapiens
tgaggccaggggctagagaccagcctgggtaacatagtgggaccctatctctacaaaaataaaagaaataaaaatttaaacaaaattagccaggcatggtggttcatgcctgtaatcccagctacttaggaggccaaggtggaaggatcgcttgagcccaggccctcaaggctgcagtcttctatgatcgtcccattgcactccagcctgggcaacagagcgagactctctctctcaaaaaaaaaaaaaaaaaaagcccacaaacagttgaaggatcaggaagagggtatgctgaggcckagatggagtcactggtgagttccaccaaatgtttaatgaataacaccgatacttcataaattattccaataagatagaagaggaaggaatacttcacatctcaatccgtgaagccactattaccctgatgccaaacaagacgaagatatcatgagaaaagcatagaccaatatattttatgaatatagatgtaaaatcttcaagaaaatgctagcaaactgaattcaacaacatatgaaaaggattatacaccatgcccaagcggtatttatctcagaatgtaaggttggtgcagcata

SEQ ID NO: 68 - DNA, 599 bp, Homo sapiens
ataaatagtatactttaaaatagtgaattttattttatgcaaaatatatctcaataaagctggatttttttaaaaggtcattatagcaaaccaggcaagagattgttgtagcttaaaccaggataatggcaatgaaaatgaacagaaataaacaaactgaaagaattctggggaagtaggaaagatagaacttcagaattgattggctatggagggagaatgagagtgatatgttccagataaattaagaagctaaataatttaaaatcatggaacacaaacatttgtaggaaatatgcrtataaaaagtttagaaggaaaaatacaaaaatggtaataatagctatgttatagtaagagaaattaaataactttttaaaatctcattttccagaatttctataatgtgactgactatattgcttttgtgtttaaaaatagtttttggccaggcgcggtggctcacgcctgtaatcccaacactttgggaggctgagatgggtggatcacctgaggtcaggtgttcaagaacagcctggccaacacggtgaaactccgtctctactaaaaatacaaaaattagctcggtgtggtggcac

SEQ ID NO: 69 - DNA, 599 bp, Homo sapiens
gtcattaaagaagatctacagatggaaaaaagcacataaaaagaagctcaacattgtttgtggtaagcagcttctaagatggtccccagtgatttccacattctcgtattcacactctgtgtgatcccttcctcttgagtataggttggacatagtcactcctttctaacaaatagaatatgaataaaagtgatagaaagcagctctgagattaagctacagaaagactgtggtttccatttgggttctttctagttttctttctcagttcaccagcctgaggatgctcgggcagcctayggaggtccacatgatgaagaactgaggttaaccaacaaccatgtaagtgaactggaagtggattttgcagtcccagtcagccttgagatgatttctacttgactgcaaacttataagagaccctgaactagcctcacccagcccacctaagccacacccagattcctaacccgcagaaactgtgagataataaatgtttgttgttttaagccagtaatttgggggtaattggttgcacagcagtagataacaaatatattattagtcatgaggtaaatacaaactaaagctaccaatga

SEQ ID NO: 70 - DNA, 599 bp, Homo sapiens
aacccacccctgctggattgggagaatgatttccaagggtctggaacctgcccttcctaacttccagatgattcataaacgttaaggagcactgtggcagagaagaggggaggacttggccatgaccatgttccttctcaagaaactgagaaggaagcaatgggaagagataggaaatgccccgaggagatttttgtgtatggttttgatccaaacgggtaaacttgtgacctgtgtactcaccaaatcatttaatgtgaactcactgtggactcacagaatcttcaaatgcattattcraagagaacatttatgaaatagaaatttaggggtccttggtttgcgaaaatcttagaatcatgatcttggaaattctaatctcatgatgctaggtatttggtctcggccacaggagtagcttgatattaacaaagtttgagaaaaccatcagaattctagaagaatgaggtcacagacacaagacactgaagaacagaaaatcctgagacaagaaggcttttagccatagtcgttgcagcttaaaagtagaggatcataaagtcatgggactaatcattacagcatataatcacaaacat

SEQ ID NO: 71 - DNA, 599 bp, Homo sapiens
ttactctcagaagataaagcccccagcagggacactcataaaaaggaatccgaatctggagtgcttaggagattgcctgctctgtccatcccctgaaatgaaagaatgatggagccggcccatgggtctcacccactagatggcgctgtgaggtgcagtgggaagagtagctggaggcaaacctttgagattcagattttggacaaatatttaacttcctggaggctcagattcctccttgagatacacttggcctgtatcccccacaacacaggtatgtagatcatacgggataacgtrtaagaaagcactttcaagtataacacgtggtgacatacttgagcctgtggactgttgtcactgaccactccaccctgtgtaccagaggctggggctgtcagagtctagtgtcccaggaccttccccaggcttctctatgatcccttcactcctaccttcaccacgtgtgaccaagtcccctggtttcccagctaggagagattgccagaaaggagctcatgttgatactgtacttgcttgctctttggaaaagcacgtattgattaatctcttcacctttgctgtaccccttttcctag

SEQ ID NO: 72 - DNA, 599 bp, Homo sapiens
gtgaaaagatagtttgagcccaggggcagaagttgtagtgagctatgactgagccactgcacgccagcctgggtaacagagagagactctgtctccaaaaaaaagaaagaaagaaaaaataattagctgggcatggtggcaaacagttactcaggaggctgaggcaggaggatcacttgagcctgggaggtcaaggctgcagtgagccgtgattgcaccactacactccagcctgggcaatagagcaagactgcctcaaaaaacaaaacaaaacaacaaaaacagaagccattcatacaytgaattttagggaccccctagatccccctgaagtctgcttatctagccaggtaagccagaacagagtggtagaaaaaggctatcaatgaagagaaacacaataaaaatattacctaggtttataggtcattgaagagttcagaagacactttcccaagcattatcctggcaacaactctgtaaggtcacagagctagaggcagggtgcagtgacttgcctgaggtcacgagctagcccatggcagggcagaaagcgcttgaagtttctgaggccaagcccagtgctcttcgtatttccc

SEQ ID NO: 73 - DNA, 599 bp, Homo sapiens
gcccaggggcagaagttgtagtgagctatgactgagccactgcacgccagcctgggtaacagagagagactctgtctccaaaaaaaagaaagaaagaaaaaataattagctgggcatggtggcaaacagttactcaggaggctgaggcaggaggatcacttgagcctgggaggtcaaggctgcagtgagccgtgattgcaccactacactccagcctgggcaatagagcaagactgcctcaaaaaacaaaacaaaacaacaaaaacagaagccattcatacattgaattttagggacccsctagatccccctgaagtctgcttatctagccaggtaagccagaacagagtggtagaaaaaggctatcaatgaagagaaacacaataaaaatattacctaggtttataggtcattgaagagttcagaagacactttcccaagcattatcctggcaacaactctgtaaggtcacagagctagaggcagggtgcagtgacttgcctgaggtcacgagctagcccatggcagggcagaaagcgcttgaagtttctgaggccaagcccagtgctcttcgtatttcccatcttgcttcccaggag

SEQ ID NO: 74 - DNA, 599 bp, Homo sapiens
ggagcgagtacctcggagacagtgcgcgccgcccggggcctcatccacagcggggtcgcgggcggcccccttagcccgcgtgactgtgggatcctgagaacattccgctgtatgtcttggaaccttctaaccctaggaatttgcaaacccctcccagtccctcatctgggggctcagagactggaggtgccttttttatttttttcttcttcactttatgaacacagaaaaatcgtttgtccctctccgggccctgcacccgccagcgtcgtgtgcaggcgtccccgggctgtggataaytagacacgttcttccctcattgcccaaggctcgttagaattcgccctagagctgtatcatgtattttctttcaaattaactttgcttgcaattaagcttagggaaccagcaacaaaagcaaacttggcccgaggtcgttcaccgcgaaaatggattagagaaacttcttccccgatttaaggggaaagattcctgcggccagcgctttggggaaagtgccccgaccgcagaggcgacgacaggggagcaggaagctgctcacggtagtcggcgttggcggcagcggtggccttcctcat

SEQ ID NO: 75 - DNA, 599 bp, Homo sapiens
attgcccaaggctcgttagaattcgccctagagctgtatcatgtattttctttcaaattaactttgcttgcaattaagcttagggaaccagcaacaaaagcaaacttggcccgaggtcgttcaccgcgaaaatggattagagaaacttcttccccgatttaaggggaaagattcctgcggccagcgctttggggaaagtgccccgaccgcagaggcgacgacaggggagcaggaagctgctcacggtagtcggcgttggcggcagcggtggccttcctcatctgggcgatgtgggctcckagaagagtaaggataacatcctggaaatgacttctgtacggtttgagcccaactgcacactcatgacttggagctgccctgtggagttacagtttaccaaacacattcatgaacataatctcatttactaaaaactttgtgagaattttcttttactaaaattttttcttattacaaaatcagtagtgttcattgttttaaaagaaataacaggtaagaaaaataaagagggagggaaataacagcaaaaaaaaaaaaaaaaaaaggctggcgggggggaacccatagttggcccagag

SEQ ID NO: 76 - DNA, 599 bp, Homo sapiens
ggggtcccaggggaggccacgggccgcggggcgggcgggcggcgccgcaagcgccccctgcagcgcgggaagccgccctacagctacatcgcgctcatcgccatggccatcgcgcacgcgcccgagcgccgcctcacgctgggcggcatctacaagttcatcaccgagcgcttccccttctaccgcgacaaccccaaaaagtggcagaacagcatccgccacaacctcacactcaacgactgcttcctcaagatcccgcgcgaggccggccgcccgggtaagggcaactactgggcgctygaccccaacgcggaggacatgttcgagagcggcagcttcctgcgccgccgcaagcgcttcaagcgctcggacctctccacctacccggcttacatgcacgacgcggcggctgccgcagccgccgccgccgccgccgccgccgccgccgccatcttcccaggcgcggtgcccgccgcgcgccccccctacccgggcgccgtctatgcaggctacgcgccgccgtcgctggccgcgccgcctccagtctactaccccgcggcgtcgcccggcccttgccgcgtcttcggcctggttcctga

SEQ ID NO: 77 - DNA, 599 bp, Homo sapiens
tgaccctgccacccgtcggcccaaattccagacccagccttgtgaccttgaagagtgcaggtattgggccgccctcctctccctaagaatctttgcaggtatggagttcaagggttctccttcattcagctagacaagctggctgtcccaggtgcagaggtcaggatgacttgcctggttgagaagaatgggaaagtaagagttctccacccaggagttttgttctgggctgcagaagagcccaggcgtgggactggggctggagaaggctggtttttgcccttagtgccagagttgagsctttctctgtttcctaatgagcaagtccagtgctttgacagcaccactaatcttaagccagtcccttctcctctcgggtcgtcatttgtgaaatgggaatttgtgactgctacctcagataattttcctgaagttaaacgagaggagaggtgaaaagcgctttgtaaactgcaaagcgcagagcatagcagggctgaaaggctttaaataagcgctgccttgtaacaaagcctcctggagtgtcatgataaagccccgggaaggagaaagacagggtgggaaagagcggacattttcgt

SEQ ID NO: 78 - DNA, 599 bp, Homo sapiens
cgacagcccttctcggcctcccaccggccgacgacaccaagagctcgcgcaccggccgggccaggtgggaatgggcgccgcgcaggggagcgagtacctcggagacagtgcgcgccgcccggggcctcatccacagcggggtcgcgggcggcccccttagcccgcgtgactgtgggatcctgagaacattccgctgtatgtcttggaaccttctaaccctaggaatttgcaaacccctcccagtccctcatctgggggctcagagactggaggtgccttttttatttttttcttcttcaytttatgaacacagaaaaatcgtttgtccctctccgggccctgcacccgccagcgtcgtgtgcaggcgtccccgggctgtggataattagacacgttcttccctcattgcccaaggctcgttagaattcgccctagagctgtatcatgtattttctttcaaattaactttgcttgcaattaagcttagggaaccagcaacaaaagcaaacttggcccgaggtcgttcaccgcgaaaatggattagagaaacttcttccccgatttaaggggaaagattcctgcggccagcgctttgggga

SEQ ID NO: 79 - DNA, 599 bp, Homo sapiens
aggattccaagactcagaaagtactcaaaggaggagatgaagcactccaccccatctctgtccccttgccaccctgccctctctgctctctgtggtcagagccacaagacagcgctggagtgctagctggggttccgggccagaaacaggaggcttctagaagttattctggcatctctaatctcaataacacattttttcaaagttctttctctttgaccagggcataggaatcacctaagggtcttatagaaaggcaggttctggtctaggaggtctggggtgaggccaaagaaccartatttctagcaagctcccagggaatgtccttgcagccgacctccatctcttgtgtaaaccaacagggagggcttgcctccctttgcccccaggccttgctcctggcccttgcagaagtgcatcttgcattcaagtgaccaaaagaagaggcattagaaaatgtgaaagggtaaaaggggacctgcacccagttttataggtggggaaacaggcccactccatgccagaaggaatttgttcaagacctcacagcaatttagtgacaaagtcaagactcaggcctcctgtataggggaaaa

SEQ ID NO: 80 - DNA, 599 bp, Homo sapiens
ccccatccttcatggtacatgcccctgtctgaactgtttttcatcagggacatattcccccagctatcttgtcatgtctaccctcaggaacaggctgagcacacacagaaagtcaagctcgtatgtgttcaaggcaggggagggttccttagggcactgaggaggaccttaggcttttactagaggatcctttggcccagactgacttggagacagggaccccttccagttggccattgtgtcctgaacagaaagcttaatttttctcctaaaccctcaactacctcagacttggggctrgagctgaggggtcagaggcaaagtttattctatactcaggcaattgactgttgctaagggagggtatagagcttgaacccaaggtccctacagtgcctcagcccgaaatcaggacgtggcttcctgccctggctctgtcaccccagctttcaagatgttttcagatatactcttgttgaatgtcattgtcacagtcacccatacagctggaaacaggagatgaggtacagtctaagggctaggattccaagactcagaaagtactcaaaggaggagatgaagcactccaccccatctct

SEQ ID NO: 81 - DNA, 599 bp, Homo sapiens
atccgagcttccgttctcagaagagggagtgtggggctggcagaggggttcaggaactccacagggagcagacagcattcctcttttgaggccaagcattgaggaccatgccaggggctctgcaaaggctggctttggcctcagccagggagaggagatgctggagagagggccggggcaggagagcagaactcactaagggcattccatctcgcccctactctgcctgtccatcccatttaaaattttgttcactgcttagtttggcctgaggagcactaactacactactttctctcraggtagttctaagctatttatttaacaggtaagtccattcctcaagctgcagtccatccccactccttagcccctaccttcacatcctcaccagattacttttacttccatattaccttcattaagatgataatcaaatgcagtctccattgggggaggcagaggttctagtccagcttggacacttgtaactgtcatatcttgggcaagtcacacaccccttctatggctctatggaggctccacacacactcacaatattcctatttatttctattgggaacttatacaagcaatga

SEQ ID NO: 82 - DNA, 599 bp, Homo sapiens
gaggtgggagttgagtcctgggaggtggtggctctcaatttattgcctggttgcagagcacctccatgcctgagaaaccatgactggggctctgccctgaccacccccacttgagaaactgctccaggttctcagatagcgagataaactggtgctctgccccatggctgagatccaccctgattctggcttcccaatctcttcctcaccctgggaggggctgacagctgttccctgggtgtctgactaggtgcttcctggttaacccttctgggctgagctggaaatccgagcttccgytctcagaagagggagtgtggggctggcagaggggttcaggaactccacagggagcagacagcattcctcttttgaggccaagcattgaggaccatgccaggggctctgcaaaggctggctttggcctcagccagggagaggagatgctggagagagggccggggcaggagagcagaactcactaagggcattccatctcgcccctactctgcctgtccatcccatttaaaattttgttcactgcttagtttggcctgaggagcactaactacactactttctctcaaggtagttctaag

SEQ ID NO: 83 - DNA, 599 bp, Homo sapiens
acatttttaattttaccttttctagtagatgtgaaatgatatctcattgtggtttttatttgcatatccataatgaccaataaggttgagcttcttttcacatgctgattggccacctatataacttctttggagtaatgtctgttgtagtcttttgtccatttttaattgggtagtttgggtgtttctttgttcttcagttgtaagaattctttgtatattctggatattaaactttatcaaatgtgtgatatgaaaatattttctccctttccatggattgtctttttactcacttgrtagtttcatttgattcaagaaagtttttggttttgagaaggtacaatgtatctatttttgctgttgttgcctatgcttttaatgtcaaaaaatgatgttaagaaaccattgccaggccgggtgcagtgggttcacacctgtaatcccagcactttgggaggccgagttgggtggatcgcttgaacccaggaattcaagaccagcctgggcaacatggcaaaaccctgttgctacaaaaaatacaaaagttggccaggtgtggtggcgtccacctgtagtcccagttactcgacaggctg

SEQ ID NO: 84 - DNA, 599 bp, Homo sapiens
gagacccttgtctctacaaaaaaatttaaaaatagtggggcatggtggcatgagcctgtagacccagctactcaggaaggtgaggtgggaggatcccttgagcctgggaggttaaggctgcagtgagctatgatcaggctactgcactccagcctgggcaaaagcatgagactcatctctaaaaaaaaaatcaaaaaccaaaaatcaaattaaagatacctccccaagaagttctaagtaacatcaaaggcaattacaaatcagtttggtttaggagtacagtgtcatcagccacaaatkactctgcacaagtatccacaggcagcagaactcatttcaaaactattcaagactatttcaatcagctcatttcgatcagctattaagatttacaatcatgattaggaaagcatgtatcatgttgacaggtgtctaatttggttatgttgatcataaatcctcagttatctgggagcacatcattaaaatcacctataggcaatacattcttacaactgtttcctatgtggtactacagaggatccatgtataattactgtatttctaaaactataatacaataatttatttatgtctta

SEQ ID NO: 85 - DNA, 599 bp, Homo sapiens
tcatatatatatacatttgtatatatacttgtataccaaaaaaaattttgttttagagacgggcttttcacgttacccagactgttcccaaactcctggcctcaaacgatctgccaccctcagcagcccaaagtgctaggattacaagcatgagctgccgtgccccaccaggaaacttattttttacaatataaacatccctattatatatttaattgagctttaaaacttatgacttgccccttctccatcacggaggtagggcttgcttcaataacctcacaccatgattataattgyattttcatatatatatatacttttttttttttttttttttagatggagactcgctctgtcagccaggctggagtgcagtggtgccatctcggctcactgcaagctccgcctcccaggttcacgccattctcctgcctcagcctcccaagtagctgggactacaggcgcctgtcaccacacccggctaatttttttatttttagtagagacggggtttcaccacgttagccaggatggtctcgatctcccgacctcgtgatccgcccgcctcggcctcccaaagtgctgggattacaggc

SEQ ID NO: 86 - DNA, 599 bp, Homo sapiens
atttttagcagagacggggtttctctatgttgaggctggtctcgaactcctgacatcaggtgacccgcccacctcagcctcccaaagtgctgggattacaggcgtgagccatcgcgcccggccaacacattcttttatcccaaatttttatcaaaacctcacacaactattcaaccttgtgtgaagtggggtccgccttcattttccctgcacaggtgctcataaagaatggtgtcctctcctggggactgtgctcagggcttttgatggcagggccatgttgaggacaactgtccagcrggcaactccagacaggcatgaaacaatccaccctctttctgccatataaatatcagcttattttgagaagaaagtgtgccagacactgctagttgtctacccacccattttcttcacttcttgtttgctcgcaagaccctgattttgttcaggtttcagttggttgtgtcttatcccttggcagtgaatcttgacttgcataaggtaagcgcagtcactatcctatcactcttgtcagagattgatttggaaacaggtacaggatactttctgggccaatgagatgttcaggaaagtct

SEQ ID NO: 87 - DNA, 599 bp, Homo sapiens
cctcacaaaggacacttgaagttgtgcccacagaggcaaatgctcatatggagactcaagaaagcctggggttcagaagaggcttgagatcatgttatgagaaacagctgaagaaaacgggggtgctcagcctagggaagaccaaggggtggtatgacaggctaacttgggagggcaaccagcctgtgtggcctctctagtgggcagagctgtcggaggacagagtggatctcacgttcaggaagaatgttttaataatgagcactgcccataaagaataagctgcacaggtcaggggtrtatacctcatttaggcagggcctggaaaggccagatagtgggaacattgaagagactcaggaactggcagaaaactgagccagattatatttaacatcctttttattatttttgtgggttttttttgagacagggtctcactctgtcgcccaggctggagtgcagtggcacgatctcggctcactgcaacctctgcctcccaggttcaagcgattctcctgccttagcctcccgagtagctgggattacaggcatgcgccattaccacccagctaatttttgtattttaagtagaggta

SEQ ID NO: 88 - DNA, 599 bp, Homo sapiens
caatccaccctctttctgccatataaatatcagcttattttgagaagaaagtgtgccagacactgctagttgtctacccacccattttcttcacttcttgtttgctcgcaagaccctgattttgttcaggtttcagttggttgtgtcttatcccttggcagtgaatcttgacttgcataaggtaagcgcagtcactatcctatcactcttgtcagagattgatttggaaacaggtacaggatactttctgggccaatgagatgttcaggaaagtctgctgtggacttctggaacacattyatttggtcttaacaaaaaataggggagggcatggtggtgtatgcctgtaatcccagctactcaggagcctgaggcaggagaatcacctgaacccgggaggcagaggttgccgtaagccgagatcgcaccactgcactccagcctgggtgacagagccagactctgtctcaaaaacaacaatgacaataacaacaacaacaacaaacaaatgtatcctaactgatacagaaagtgaccatgaaagggcaggggaaaactgattcttgatgtattccctttgagacttctcttggcttctt

SEQ ID NO: 89 - DNA, 599 bp, Homo sapiens
aactccagacaggcatgaaacaatccaccctctttctgccatataaatatcagcttattttgagaagaaagtgtgccagacactgctagttgtctacccacccattttcttcacttcttgtttgctcgcaagaccctgattttgttcaggtttcagttggttgtgtcttatcccttggcagtgaatcttgacttgcataaggtaagcgcagtcactatcctatcactcttgtcagagattgatttggaaacaggtacaggatactttctgggccaatgagatgttcaggaaagtctgctstggacttctggaacacattcatttggtcttaacaaaaaataggggagggcatggtggtgtatgcctgtaatcccagctactcaggagcctgaggcaggagaatcacctgaacccgggaggcagaggttgccgtaagccgagatcgcaccactgcactccagcctgggtgacagagccagactctgtctcaaaaacaacaatgacaataacaacaacaacaacaaacaaatgtatcctaactgatacagaaagtgaccatgaaagggcaggggaaaactgattcttgatgtattcccttt

SEQ ID NO: 90 - DNA, 599 bp, Homo sapiens
gaaggtctggtaaaggtatttaacatttctgaacttcaatttcctcatcccaaaaatgggggataaattatattacttaagacagttaaggcgattgtatgaaatgagtgtaaaaatgttttaccgtactacctggcaaacagcagacacttagtaaatattgtttgttgggcacttgataaacgtctctcaagttcctactgtgtgccagttgttgtgttagctgcaaaggatgtggagatgagattctgtctcttcctttaaggaaaggggatggtgttattttcttcctaccagatkacaagttaatactatttaaaacatgatttctgttaatttgtgggagattgcatttttttccccatcagatcattgtggtaaaataacttgtgctcttttcttattccgtggtttgttttgttttgttttctataggattttgtttgtttttcacaaaaatggtcatttgagctgtaaggcaaaagtgcagcctcctaggctgaatggtgcaaagactggagttttttccacaaggagccctcatcgtcccaatgcaataggactgaccctggccaagctggaaaaggtagaaggtaacc

SEQ ID NO: 91 - DNA, 599 bp, Homo sapiens
agtattcaactaatatgtattaagcaaggagcgtgacaagtgctgggatatggagttgaacaggaaaaaatccctgcctgcaaggagcttacgttctggtgcataaacagacactctataaacaatgtggacatgaagtggcagagatcagcactaggtagagaggaaggagctcccccaagccttggccaggagggttggcaggccctggggggaggtggtgtttgaactgagtcatgagggatggataaatagaaatctttcacaagaagtgggcagggcattccaggaatgagctaraaagcccagagtccaaagcagcatgacatgtacaaaaactacacatggcacaggtaaaatgtgggtgggatgcggagacagaaaagggtcaatagggacacagctcacatgattggaagatgagaccctggatagactccagtaaaacttatgtgaatttcagagaactcctccctccttgcaataattcacaatcctattcccaccaccatgacacccagtggtatcctcagagtaggagtcagcttactgcttggacttaacctaccttccccagaaagcagagcttgaggcagagg

SEQ ID NO: 92 - DNA, 599 bp, Homo sapiens
accaaagcctatacaccaatgttcatagtagcagtcttcacaatagccaaaagataggaacaactccttttaatcacatttatcagccaaaaatagtcacatgaccatgctttatttcaaggggacagaagagtatatgcatccacatacttagaagtaaaagagaattggataattggtgaacattaactatcttccacgattacctgtgatactaaaaattatttccacaccaacatattgcaaagagattgaaacagaacttgctcaaagaatgtgaactgtcccatgaaccagagrtcctacccagaaaaagtgggagaactgcaactccagccacaccttattctctcaatacattcccagtatggacggccaaaattccagaaaccccaaggacatctgggtggcagctctatttgattaaaaagtctcttaactggcagaggttgccgtgagctgaaatcgcgccattgcactccagcctgggtaacaagagcgaaactctgtctcaaaaaaaaaaaaaagtctcttagctgcctagaaaccaacgttcttccttttcacttgctatgcatgctatcagatacctctaccac

SEQ ID NO: 93 - DNA, 599 bp, Homo sapiens
gaaagatcaagggaattcctgtagtgttggtgaagggtgatcctagggtgataaccatgcccaaatcagagaggccaagagacctagattgcagcaagacagaaggcaagaagtggaaactggtaagttcatacgaaaacacagagaggagggcattaaggatggtaataattttttttaattcagcaaatgaaaacacagataattattatctccagacaaaaccaaaagtccaaaaatggaaataaataaataaataaataaataaataaataaatagtattctactagttcagctgkgaaaagcatttagataatactataaacactgactattgatctaattaacattatgatatgactatattaggaggatggggtaataggaaggatgggtacacatggtggaaataggtgtaaaagaaagctaaatctttattttccatagtgttaagcccaaaatctgaaatattaagtagtagctgacaccgaaagcacatgcaacaaaagcaaaaataaacaactgggactatatcaaattaaatagtttcttcacagcaaaggtaaccatcaacaaaacaaaaagacatccaacctat

SEQ ID NO: 94 - DNA, 599 bp, Homo sapiens
ttggtagactacacatacctccctgtggcagccaggcccccaccacccattctcatcattccacacaaacgtgagcccctaccatgaacaagatgttgcatggagcactggcagaagcagagtgagttgaaggtggcttcctcccaggactgcatggtcacagaccccagcaccagcaaagtatggtcatcaggaccacaggaagggccagagatgggctatggggcactggggtcaggaagagtgccccatcttcctgaagggtgatactggacctggtccttggagaataagaaagayctgatgaagcaaaaccctggaaggagggtatccaagatgaagagaaagtatgaggtgcagtctggaggcaggaaagatgggctgcatcggaatcacaccaggtagaatggttggttggttgaattgggaggtggagctggagaggtatgtagggcagatggcaaaaggccttaaattcaggccaaggagagtggacttgattatggaggcaacagagactgagggacaagagctgggattggaaagaggccccaagcatatatgaagctcccctactcatgcctgccccacaccttttc

SEQ ID NO: 95 - DNA, 599 bp, Homo sapiens
gagtcaaaacagagactcttccaagtggcatgggaattagggtccccttgacctgaggagcttgggtttctggagatgactgaagtggacaggaatgaaaggcatcctgaaattagtcttaagagtctcttaggggaaggtggacttgagacaaaaggcacaatgctggttagcaacaattctctactgtgcctgtgagtaatgcagaagccagaatgagtccctgaagggattgttctcttaaggggagttcactgtgaaggcttccggggagggtccatctaccctgaaaatggatcyctagggttgacttttccaactcctcaggtggtgcagtgttatgatgctctggagcacaaggagctcagagcctgccactgtgctgctgggtggtgtggtggtgtggcgggcaggctcccaaatcattccacacaagtgacatttgtgtaaacaccactgtaatttctctgttggagtctgcagacccatcctcaagttatcagggagaattttaatttttctatattgataataagcttaacaaataataaaatcatattctatgtcctctagatgtgaggctaccagataaattacag

SEQ ID NO: 96 - DNA, 599 bp, Homo sapiens
tacagaaaaggaaactgaggtcacaagatgggcagacacttgtacagtattatacagcattagtggcagggccaagatcacatcccgacctctgcattcctgtgccagtactcttttcctcaccattcctcctctgaagagcgcttccttaaaaatagttctaacaaaaatgaagctgcttcttcatgtcattttactgatctcatgtaggcagtattgtgtcaggaatcttatatttgaattgtagcaacgtttggtttcctggatatctgcttcagaggtcatgtggtgaaccctccragacaccagtcaaagtttgctgaacccaaacctccaagtcgttcacctgaaagatctaaaccaacagaaatgaaacaaattggcattgctttgtctcactatctcgtgagatgcgtgagaattcttaagcagacagttactgcctgtgtcatgggtaaaatcccaaatcattttgtggggtttgttattgttccaacagctataagagagccaatatctgcatgtaccaacatctctttgtagtatcgcttcctggatccttttttttttttttttttttccgcttgatttgggagtca

SEQ ID NO: 97 - DNA, 599 bp, Homo sapiens
gacatgagctgagagttgtgaataaacccctgtactttgtacttacttactcaacttgacaaacattccccatggacttgccctctgtcagactgcactgggaactggggattctgtagtgaatcaggcactatgtttgctcttcagaatctagtaagagttgggctcacatagccaaaatgcaaagcagagcatggcagcgccctggaagatgaattccagattcctgctgcctggtccagaatcagcatagcccaacaatgctaggtgaccttggtcaagttgcttaatctctctacyctcagtttccatccttacccttctttctcaaagagctactctaagaagaaaataagacaattggcctggacatactttggagtcaaagctgaatagggagagacgattgctagtgtttcatagtgtcatcagtaacatagacaccatttattcagtatcttcccaggtaccaagcatctgaccagtgttgcatgtcattcttccaacactcctggtacagctctattattatctatattttagagatgagaaattgaggctcagagaggggaggtgacttacccatggtcacactgctt

SEQ ID NO: 98 - DNA, 599 bp, Homo sapiens
aaaaaaaaaaaaaaaagagacgaaactattatgtggttgtgctgccctgccctcagtatgttgtgtaataaatataataggggtatgaataattgtgcaatgaatacatcctgatggtaaaaaatttaatgtacacacaaaagggggtaggcagataggagggaaacaatattgtaacatcccactgtactattggttctcaaccctacctccactttaggattgtcaggagagcttttaaaataaaaacatcctaaatgcaattctatatcctggattggaccctgaagcagacaaagsacattagtggaaaaattcatgaaaacggaataatgtctgtagttaatggtagtgtaactatcctgatttcttagttttgacaaatggcccatggttatgtaagacgttaacattggtgtaaactgggcgaaaggtatatagtaacttttgtactatcatgacaacttttctgtaattctaaaattattcctccaaaaactattatttgaagcataaaacaattaaaaaataaatatacctatgcctgtacatcagctagatgggggaatctgaatccctgggggagaagtctggacata

SEQ ID NO: 99 - DNA, 599 bp, Homo sapiens
acacacacacgggcagcctaattttccatccaaagtctttacattcagaaagcagattaatcttggattcacttgatttcccatggggatctctcatactaatccttcagaaaagtgaaccaacttaaatcccccaccaagctctgttctaaggaggaatattcaactctgtggcaattacattccaaagtccctattttctgtagagagcttccctggtgttaaccacttttccttagttattcttttatttttttatttttattttgttttattttattcttttattctaccaacaaytccacaatgtaggtattagtgttcttttgctctgtgagaaaactgaacttcaaaaaggttaaataactttctcaaagacacagcaataagtggcagaaccaggattcaaacactcattctcaggacaccagaccttgtacctcagtgttccctggtccaccctgccagaccagagcagaggtcctttgtacaggttgcagggaagcttttttgagactcacaggaaatgagagctaaaatgaccttgtaggtcctcatttcacatttcgttccatctttcaaacaaaccacattgtaca

SEQ ID NO: 100 - DNA, 599 bp, Homo sapiens
agtttgatttccgtcactcaaaccaagagagtcctaaatgagaccagcatcttccatttattaagctcacttatgtgctgggagcttttcactgtatcactttatgtaatcttgcacacagtcacatgaaccatatggccattttatagacaaggaagctgagacacagagggctgagggatgtacctagggccacccagctgttagtaagtggccaaagagggattcaaacccaggtctgcctaacaccaaacccatgtccctaactcttgccacagtgctattcacactatgccctgragcttctccagttctcactctctgggatgggcagcttcagaacagactcagcttccatgacttgaccttttgtgtactggcttcttgtaccgcgtatgaactgcacataaaattactcccataaataatttttcaatacaagatcttatgatggttcttagcagcttagcgggttagagtcaaagtctccatgctggatctgtcctcacaccttcacccaactaagtaaatactttttattgacatcccctttggaaagtgagacttttctgatctgatcaaatcctggccttaagaac

SEQ ID NO: 101 - DNA, 599 bp, Homo sapiens
gtaatggtgagcactcaggggcagaatggccaatgtgtgagacttcaaaccacacaagaggaagagaaaggcaagcatccccagctagcctgaggcatattggcccatgatccttagaggcagaatgtggagggagctccgacgggttatggccaagtatgttcttgggcaaaccactctccaaagaagggaaagagagaagctccctgttagtaaactaccaccatgtgccaggcacttttcctgctgtgtctactctgttcagcaacactgtgaggcggggattatgatctcttatcsgttcctctggtgacttctaaccaaggctgtggcaaggttttgtgctataggtggcatttgccctgagctttaaaactaggtagactttgggcatgtagaggtaaagttgggatgtggaagggaacaaagggcattttaggcagagggaataaaatagagagggaaaaaaggaggtaattgtgggtccctggattttaaaagtagagttgccattgtttcattttttttaaaaaagagtatactctgaggagtagctggagctaagtctgcgaaggccaattaggatctgttcttgaaaa

SEQ ID NO: 102 - DNA, 599 bp, Homo sapiens
caggggcagaatggccaatgtgtgagacttcaaaccacacaagaggaagagaaaggcaagcatccccagctagcctgaggcatattggcccatgatccttagaggcagaatgtggagggagctccgacgggttatggccaagtatgttcttgggcaaaccactctccaaagaagggaaagagagaagctccctgttagtaaactaccaccatgtgccaggcacttttcctgctgtgtctactctgttcagcaacactgtgaggcggggattatgatctcttatcggttcctctggtgacktctaaccaaggctgtggcaaggttttgtgctataggtggcatttgccctgagctttaaaactaggtagactttgggcatgtagaggtaaagttgggatgtggaagggaacaaagggcattttaggcagagggaataaaatagagagggaaaaaaggaggtaattgtgggtccctggattttaaaagtagagttgccattgtttcattttttttaaaaaagagtatactctgaggagtagctggagctaagtctgcgaaggccaattaggatctgttcttgaaaaagagccccaaatgcc

SEQ ID NO: 103 - DNA, 599 bp, Homo sapiens
atcacctgaggtcaggagttcgagaccagcctgaccaatatggtgaaaccccatctttactaaaaatacaaaaattagctagtcatggtagcatgtgcctgtagccccagctactcaggaggctgaggcaggagaattgcttgaagctgggaggcggaggttgcagtgagtcgagattgcgccactgcactccagcctgggcaacagagggagactctgtctcacaaaaaaaaaaaaaaaaaaaaaaaattaaactaactaaataaaagttaaaatttagttcctcagtcacactagccwcaaaggcctcaataaccctatgtagctactggctactgattggacaacacaaacatagaacatttccatcattgtagaaagtccttttctagaaactcaaactatggctgcagaactaagaggggcagaccatctgctacaaacaccattcttttccaacccagcacccagcactaggccagacacagcctccttcctcctctctctccttcctttccacccatctttctccacacccctttccaggagcagatcataatcccggaagagacaaccttgaacaccataatcccaaatat

SEQ ID NO: 104 - DNA, 599 bp, Homo sapiens
cttaactttattattcctctgtaagtgatcctttcattgaagtctctatttgaaccatctgggtgcattctgtttcctgttgaaagtgataaagcaaaatgcttattttctcgtgcctcagtgtcctcatctaaaaataagaatcatattaatgcccacttcgcagggatgatatggcaatcaaatgagaaaatgtatttatgatacctagttcagtgcctggcataggtaagtgctcaatgaacgttcattgatattatcatcatcactcccattggaatttataacctggggctataygatctctataggtccttacagcatgaatggaatatgaatgggtgattctctatacaaaagttgctgccaatccagatattattttagtcttttgatctcaaggtagagttgaggcttagacaataaaaattataattatctcttgttgccatttataagcaagaggactatgccttttacagagaggtttcttcacgtgatgtcagccatttggaactttcatgccctgttacagataacaggtaacatttatggagcattttctctatgccaaataacctgagtctttatatgtggaa

SEQ ID NO: 105 - DNA, 599 bp, Homo sapiens
gcagagatcagcactaggtagagaggaaggagctcccccaagccttggccaggagggttggcaggccctggggggaggtggtgtttgaactgagtcatgagggatggataaatagaaatctttcacaagaagtgggcagggcattccaggaatgagctagaaagcccagagtccaaagcagcatgacatgtacaaaaactacacatggcacaggtaaaatgtgggtgggatgcggagacagaaaagggtcaatagggacacagctcacatgattggaagatgagaccctggatagactcmagtaaaacttatgtgaatttcagagaactcctccctccttgcaataattcacaatcctattcccaccaccatgacacccagtggtatcctcagagtaggagtcagcttactgcttggacttaacctaccttccccagaaagcagagcttgaggcagagggcttttgagctgccatagatgatcctcaacttaccatggtttaacttatgacttttcaactttatgatggtgcaagagtgatacacattcagtagaaaccgtagttcaagagcccatacaaccattctgattttcacttg

SEQ ID NO: 106 - DNA, 599 bp, Homo sapiens
gtgattgcttataagacaacccacggaatccagaataagttctgcctctcaagatccttaaccacatctttgtcacataagggaataatcactcttttgctatataaggtaatagtcacaggtagcagggattaggatgtgaacgcagcttttggggccaccatccaacccactacaccagaaattagcaggtgaatctccaccccttccctgtttgatgttattttgtctctctcagtatattcctttgggtgggacacttgtgtctagaaacagcaattgtcggtttgaatctcatcyatagtgtttctccggccttcttttctttctttctttcttcttttttttttttgtttgaagcagagtctcgctctgtttcccaggctggagtgcagtggtgcaatctcagctcactgcaaccgccgcctcctgggttcaagtgattcttctgtctaagcctcccgagtagctggaattataggcgcataccagcacacctggctaatttttttgtatttttggttgagatgggtttttgccatgttggccaggctggtctcaaactcctgacctcaggtgatccacccgctttggccttc

SEQ ID NO: 107 - DNA, 599 bp, Homo sapiens
tggcaaaaacccatctcaaccaaaaatacaaaaaaattagccaggtgtgctggtatgcgcctataattccagctactcgggaggcttagacagaagaatcacttgaacccaggaggcggcggttgcagtgagctgagattgcaccactgcactccagcctgggaaacagagcgagactctgcttcaaacaaaaaaaaaaaagaagaaagaaagaaagaaaagaaggccggagaaacactatggatgagattcaaaccgacaattgctgtttctagacacaagtgtcccacccaaaggaayatactgagagagacaaaataacatcaaacagggaaggggtggagattcacctgctaatttctggtgtagtgggttggatggtggccccaaaagctgcgttcacatcctaatccctgctacctgtgactattaccttatatagcaaaagagtgattattcccttatgtgacaaagatgtggttaaggatcttgagaggcagaacttattctggattccgtgggttgtcttataagcaatcacatgtgactttataagacagacacacagagaagaggaggaagaagcaatgtgaccacag

SEQ ID NO: 108 - DNA, 599 bp, Homo sapiens
gagggccctactccctcctgaggctctaggggagggtccatcccttgcctcttccagcttctgatggcctcatgcattccttggcttgtggccacatcactccaacctctgtggtcacattgcttcttcctcctcttctctgtgtgtctgtcttataaagtcacatgtgattgcttataagacaacccacggaatccagaataagttctgcctctcaagatccttaaccacatctttgtcacataagggaataatcactcttttgctatataaggtaatagtcacaggtagcagggattrggatgtgaacgcagcttttggggccaccatccaacccactacaccagaaattagcaggtgaatctccaccccttccctgtttgatgttattttgtctctctcagtatattcctttgggtgggacacttgtgtctagaaacagcaattgtcggtttgaatctcatccatagtgtttctccggccttcttttctttctttctttcttcttttttttttttgtttgaagcagagtctcgctctgtttcccaggctggagtgcagtggtgcaatctcagctcactgcaaccgccgcctcctgg

SEQ ID NO: 109 - DNA, 599 bp, Homo sapiens
tctaacgcctgtatttttcaatttacattgaagacgccattgcatggctgctatgggaatttttgtactggggacagaaggcatcatgggtgaaggacagcatgaacaaaggcccagagccccgtgtctgcagcccaggctcatctgcttccctcaggatgctcacccctctctctttaggggttccaaagttggcttactgatgttatataaccttgatttctatcaacacctaaaaaataccagttatttccactggcagccagtcagaaataaccccagaatggcaagaatgcagcygtgtctcttaggcatgtcagcaaagggcatgctgactgagtccaagcagagaaacgcctgagctgagaagcagcccctacaggacacatgggcagcagagacatgaattcaaaagatgggaattccctgcatagccgcacaccatcacccactgtgagtggcacccaagtcatctcatgtactcctcacagcagtcctagaggtagcaaacctcatcatccccattttacagtggagaaagccaaggccctccgagcttggggagcttgcccagagagtctctatgtattggaactggg

SEQ ID NO: 110 - DNA, 599 bp, Homo sapiens
agagtgatgtgtacatttcaagaatgtttacccaattcagggaagaaccatttgccactaaaaggtccccccgcctgtcctcatcaccatgtcaccctgcagaaggtcaagtggccttatgcatgaggctaattccctgcattcctgatcaaggggcagggagggccaggcaaagtcactgctgtcttcagcagacagtggccatgatggaattaggaccagaggcccacagttgcaaagataactcaaataccctgtaggcttccttcaaaggctgaaaagcacaggcttggggcttcrttcattggaacaaaactttacgaaaagaaacaggtacactgatgtctaggcatctttaagacaggtaacctcaaatgacatttacatagcatgttatggtttattgtatacatctcaccagcctgctaaaaagaaaaagggtctctcctgaaggctgagttccagccccagctcagctactgacttgctgggtaatcttgggcaggtaacttactttctctgggtcccaagttcctcatttgtaaaataaggggtggagtaggtcatgtctaaattggtccctgattcttaccttgtaa

SEQ ID NO: 111 - DNA, 599 bp, Homo sapiens
accttattttaaacagctaaagccacccaaatctctatttctgttctctcatacctagagacacaaaaaaattcacaaaaaataataaaacctagttagaatgtaaaggagtcaatcagttttcacttgcagcagattaaatggtctagaatgaaatgagaggcatggagaggtctcctggcccctcagacgtgctctccaaatgtggaggccagttctttgcatcactccacagtgcatccccacacagatggcagagtgtgaaagcgtattgcaacccaaatcagcatcttccctttrgtacagtatttaaaaggactggccaggtgcaggtgctcatgcctgtagtcccaacactttttttttttttttgagacagagtctcactctgttgcacaggctgggattcagtggtgccatcttggttcactgcagtgtccgcctcctgggctgaagcaattctcctgcctcagcctcccgagtagctgggattacaggcacgcatcaccacacctggctaatttttgtatttttagtagagactgggtttcaccatgttggccaggctggtcttgaactcctgacctcaaatgatctac

SEQ ID NO: 112 - DNA, 599 bp, Homo sapiens
atgtgttctgctagaagctgcaagacactaaattgcatgtacaggccaaatacaactgcccacagccatatgcctatgaaaaagatactagaaaggatatcagggtgttttccttctttcatcccctccatcctttgcatttacagatgaggaaactgagaccaatgggctgttcctgctgttgagaacctgtccctacagtttccgtgtggcccacaggggcagagtggaggagctggtgaccatgcaacaggaggatcaggtcatgaaagacacctggagctggggtggagacagctrgttcaacacgtggcttcagcacacacccactgagcaaaggagcctactcaatgccaggccctgagggagagaggcagacccgacccagtgctcactatcacggggcacatgggaggaagagacgatctctgcctgaggtgatcagagagggcattaagcaggagtggcatttggatctcaaagcagggtgaggacatgtggcacactggagccagtttgtaccagcttttggaggctagctgcttttaggaatttcacatgccagtgcttagacataggtattattaaaaataaatttg

SEQ ID NO: 113 - DNA, 599 bp, Homo sapiens
ttttgagaatccattgtaggaactcagataggagaagacagggcaggagaaggtagaggaagatacagtagggattataatccctactgtatagatacagtgtcttctatagcagagtgacactgtaggagatgtggtagctgctatctcctgggggccagcatccttactgccctagattcctctggctcttgtccttggcccaaatagctttcaggagaaaggaggcaaccctgggtggggccactcccaacctcccactgagtgagcctggctacagactgtgctgagctggaccaycccaggagaaaatgctttggagccactggagagaggggaccctgtattttcaaagcatccctgagctggagacaaacctagcagggccaaaagatcccctcatgcgatgccagccctctctaggtcaaaacccaagtaggagaagatgaggcagagagggaaggtctgggcatgctgtgctgattccaggatccctgcacacccaccagggaacctgtgcttctgcccttcaagaacacacactgcagcccaattgtgttgttactctgcctaatcctgccattatgttgaatcctagt

SEQ ID NO: 114 - DNA, 599 bp, Homo sapiens
ggttgggtgcttctcaacggcaggagcaaaaccccactgtctctcacaggacccaatcctgagtagctgctcaaccatgtgtgtccagtgggatggtcctgggtgaacttagagttaaggctcgcagatcacagaggaaggaagaacttaaagacagcaggtaaaacacgggaagtcaacattccctcctccctctgcctctgccaccagcctatgtaatattccaggtaatgcaacaggactttgtgggtgacctagattttcttcataataatcacagccatttactcagcacatccyatgcaccccacacttcatgtgtattagctgtaatcctaacaccagtcctaatcccattttcaagatgtggggcctgaggttcagagacctcaaattatttccttacaaggagaggagctaggatttgacctcaggtgtgtttgattccaagcccgcattctctctgtaaaacatgcagccttgttcagaacactgatagtctcagcaggcttgaggctaaagcacttccctctgctggacgttttttcttagtgtagagggtagaagtagtgagggtggggctcttccctgaccctgac

SEQ ID NO: 115 - DNA, 599 bp, Homo sapiens
actgggagacaggagagccagggttcagggccactgggagagcttcccccagtccctacctgtctgcttgcccatctgtaaactgccaggcttgcatgcatctggtgtgttttcccggacggagtttgactactgatgtcctccttggctcctggggcccgctgtaaacaccacttcctcaccctggctctcagtttgccccctacagtgtggctgtccacataggtgcccgtctacctgtcaggttgggtgcttctcaacggcaggagcaaaaccccactgtctctcacaggacccaaycctgagtagctgctcaaccatgtgtgtccagtgggatggtcctgggtgaacttagagttaaggctcgcagatcacagaggaaggaagaacttaaagacagcaggtaaaacacgggaagtcaacattccctcctccctctgcctctgccaccagcctatgtaatattccaggtaatgcaacaggactttgtgggtgacctagattttcttcataataatcacagccatttactcagcacatcccatgcaccccacacttcatgtgtattagctgtaatcctaacaccagtcctaatccca

SEQ ID NO: 116 - DNA, 599 bp, Homo sapiens
ctctttattgacactatatcaatccaactttccctaaaagataaactccaaaagagatagaaagatggagcactgggagacaggagagccagggttcagggccactgggagagcttcccccagtccctacctgtctgcttgcccatctgtaaactgccaggcttgcatgcatctggtgtgttttcccggacggagtttgactactgatgtcctccttggctcctggggcccgctgtaaacaccacttcctcaccctggctctcagtttgccccctacagtgtggctgtccacataggtgsccgtctacctgtcaggttgggtgcttctcaacggcaggagcaaaaccccactgtctctcacaggacccaatcctgagtagctgctcaaccatgtgtgtccagtgggatggtcctgggtgaacttagagttaaggctcgcagatcacagaggaaggaagaacttaaagacagcaggtaaaacacgggaagtcaacattccctcctccctctgcctctgccaccagcctatgtaatattccaggtaatgcaacaggactttgtgggtgacctagattttcttcataataatcacagccatt

SEQ ID NO: 117 - DNA, 599 bp, Homo sapiens
ggatctgaaagagccctgggagacaattagcctaaagctttcatttcacagacagggatgctgaggcccagggagagacagggaactgcccagatcacagagcaagtcaagcacagatagcctagaacacaggccttctgattgccaggacccacgctccaagctcaaggcctggccttgaaaagaccaaggtgctgtcataggtgagttcttgctcttgcccctgaacttccatgtggatgtcgttttgaaaccaaaatactctgtaaacaacacttattaacacaagttcacatgtaytctgctgtggaaggagatgcgtctttcttctttgtgaaatattagctcagataggcatacaaagaactctatgactctcagtttgcttaacatatttgatttgctttatgcctacaagttcatactaagctcttgcatatctgctggcctctttattgacactatatcaatccaactttccctaaaagataaactccaaaagagatagaaagatggagcactgggagacaggagagccagggttcagggccactgggagagcttcccccagtccctacctgtctgcttgcccatctgta

SEQ ID NO: 118 - DNA, 599 bp, Homo sapiens
gggaaaagttccgttctctgttatttatatgggcagattgaatgagcattatctccgcaaggtcttcccagtactttatagtttgcaaagcaaactttaatgtctctgaactctaaggatactctggcaagcaaagatagtctctcattttactaattaggaaactgaggccagagagggaaaggcacctgctctgtgcatcatgcgtgcatgcacatgtgcctgtgtatgtgtgtttctgtgtgcaagaaaggccttgaatgctcctgcagacctggatcccagttgtgagagaagttktcagttctgatgcttggcgggcagcgttccggcccactgcccatgtatggagtcactcagacttagctaagtacacagcagccaggagcctgtgtttcagcagctaaaatcagtgggcagcacaaaaatgtaggtgtagctccctcgccgcttccaccttgcccctcttctctcccctcccacagggtggaactgcctggaataagctcagcccagataacaagtctacacatgcagcatccagggaataagcacagggcagctctggggcacacatggagacatgtatcttcatacac

SEQ ID NO: 119 - DNA, 599 bp, Homo sapiens
acctcattaaaacttcttgtgggagctcctgtggaaaatgctgtggtcaggaagttagggtagagtacctgcccttccctctaacaatagagcattcctccaaaaccagtgtaggctggaggaggccatgggctagctcaggtctagccttggcactggcaatcactgattcacattgaatatgtacatcaccatcatcatcaccaccacctactcttcttccaagaagacaaataataaccaccacttatatcaagcagtcttatatgtgcagacattcacaacagcccagtggaggasgcgctatagtcatgccacatgacagatgatgaacctaaggtacagagcaattaaatgacagggcctaggcattgcaacacaactgcaagtggcagaactgggatttgaaccaggcattctagctccagagtccattctattagtcatctcgctacattatcttccttttgaatcatgaatcagggtattctctggcaatcaaaccattccagattcggtggggaaaaggatacatcagatagcatgtttctgaggctgaaatcaccaattttgtattaatagaatagggtattatctat

SEQ ID NO: 120 - DNA, 599 bp, Homo sapiens
acgccagcactggtagagagaggtgcagagagccccacctttttgcttggaattggtgagagcttgtgcttacctcattaaaacttcttgtgggagctcctgtggaaaatgctgtggtcaggaagttagggtagagtacctgcccttccctctaacaatagagcattcctccaaaaccagtgtaggctggaggaggccatgggctagctcaggtctagccttggcactggcaatcactgattcacattgaatatgtacatcaccatcatcatcaccaccacctactcttcttccaagaaracaaataataaccaccacttatatcaagcagtcttatatgtgcagacattcacaacagcccagtggaggacgcgctatagtcatgccacatgacagatgatgaacctaaggtacagagcaattaaatgacagggcctaggcattgcaacacaactgcaagtggcagaactgggatttgaaccaggcattctagctccagagtccattctattagtcatctcgctacattatcttccttttgaatcatgaatcagggtattctctggcaatcaaaccattccagattcggtggggaaaag

SEQ ID NO: 121 - DNA, 599 bp, Homo sapiens
atcaaatcctggccttaagaacgacaaccaatattgtaatcttcaaaggctcagacaggtgaaatgacttgcctagggtcactcagcttgccagggtcagaacagaaattggaacccaattattcattttgtctagtcctaacaaatttctgagtccttatagtcctcagggttccaaaccttctgtcgcaagacctctgcatccaggcacagatgacggtggtggtgacgccagcactggtagagagaggtgcagagagccccacctttttgcttggaattggtgagagcttgtgcttmcctcattaaaacttcttgtgggagctcctgtggaaaatgctgtggtcaggaagttagggtagagtacctgcccttccctctaacaatagagcattcctccaaaaccagtgtaggctggaggaggccatgggctagctcaggtctagccttggcactggcaatcactgattcacattgaatatgtacatcaccatcatcatcaccaccacctactcttcttccaagaagacaaataataaccaccacttatatcaagcagtcttatatgtgcagacattcacaacagcccagtggaggac

SEQ ID NO: 122 - DNA, 599 bp, Homo sapiens
gaaagtgagacttttctgatctgatcaaatcctggccttaagaacgacaaccaatattgtaatcttcaaaggctcagacaggtgaaatgacttgcctagggtcactcagcttgccagggtcagaacagaaattggaacccaattattcattttgtctagtcctaacaaatttctgagtccttatagtcctcagggttccaaaccttctgtcgcaagacctctgcatccaggcacagatgacggtggtggtgacgccagcactggtagagagaggtgcagagagccccacctttttgcttkgaattggtgagagcttgtgcttacctcattaaaacttcttgtgggagctcctgtggaaaatgctgtggtcaggaagttagggtagagtacctgcccttccctctaacaatagagcattcctccaaaaccagtgtaggctggaggaggccatgggctagctcaggtctagccttggcactggcaatcactgattcacattgaatatgtacatcaccatcatcatcaccaccacctactcttcttccaagaagacaaataataaccaccacttatatcaagcagtcttatatgtgcagaca

SEQ ID NO: 123 - DNA, 599 bp, Homo sapiens
tagcgggttagagtcaaagtctccatgctggatctgtcctcacaccttcacccaactaagtaaatactttttattgacatcccctttggaaagtgagacttttctgatctgatcaaatcctggccttaagaacgacaaccaatattgtaatcttcaaaggctcagacaggtgaaatgacttgcctagggtcactcagcttgccagggtcagaacagaaattggaacccaattattcattttgtctagtcctaacaaatttctgagtccttatagtcctcagggttccaaaccttctgtcrcaagacctctgcatccaggcacagatgacggtggtggtgacgccagcactggtagagagaggtgcagagagccccacctttttgcttggaattggtgagagcttgtgcttacctcattaaaacttcttgtgggagctcctgtggaaaatgctgtggtcaggaagttagggtagagtacctgcccttccctctaacaatagagcattcctccaaaaccagtgtaggctggaggaggccatgggctagctcaggtctagccttggcactggcaatcactgattcacattgaatatgtacat

SEQ ID NO: 124 - DNA, 599 bp, Homo sapiens
ctgtatcactttatgtaatcttgcacacagtcacatgaaccatatggccattttatagacaaggaagctgagacacagagggctgagggatgtacctagggccacccagctgttagtaagtggccaaagagggattcaaacccaggtctgcctaacaccaaacccatgtccctaactcttgccacagtgctattcacactatgccctgaagcttctccagttctcactctctgggatgggcagcttcagaacagactcagcttccatgacttgaccttttgtgtactggcttcttgtacygcgtatgaactgcacataaaattactcccataaataatttttcaatacaagatcttatgatggttcttagcagcttagcgggttagagtcaaagtctccatgctggatctgtcctcacaccttcacccaactaagtaaatactttttattgacatcccctttggaaagtgagacttttctgatctgatcaaatcctggccttaagaacgacaaccaatattgtaatcttcaaaggctcagacaggtgaaatgacttgcctagggtcactcagcttgccagggtcagaacagaaattgga

SEQ ID NO: 125 - DNA, 599 bp, Homo sapiens
tcccgggatgttttttagctggtgggaaagaatcctctcctgataggtcatggaatggtgaactgagaggttttggaagtcatgtcctcaccccataggaaacctctgtggcagggcagaaggaagccaacagttagagatgagaagctgagaagagagagtcttgctgatgtctaagtccctgtccccgactgccctaaggccaaccccacccctgcccttcttggttatttaaaacaacaaaatgactactttacttaagctagtgtgagtttgatttccgtcactcaaaccaagagrgtcctaaatgagaccagcatcttccatttattaagctcacttatgtgctgggagcttttcactgtatcactttatgtaatcttgcacacagtcacatgaaccatatggccattttatagacaaggaagctgagacacagagggctgagggatgtacctagggccacccagctgttagtaagtggccaaagagggattcaaacccaggtctgcctaacaccaaacccatgtccctaactcttgccacagtgctattcacactatgccctgaagcttctccagttctcactctctgggatg

SEQ ID NO: 126 - DNA, 599 bp, Homo sapiens
agggcgcttgatgaagacatagttccaaaggagcaatgtgtccctgggtaggcaggcactttgcctttgcttggcttcagtctctccgtctgtgaagtgggccaggtggttcctgaggtcccttctaacactatgactgtttgttgttgttttttttttttctaattaccgtgctcccaggctcattgagatctgcagggggtcgacagaggaaatgaggccagggtcttactgaggcgggggctatgggttggtgaagggaaagctccacgcggagccctccagtgagactgccacagytcctgggcttcctctgctgggaacgctgcctaagctctgcaaacagcagctgaggatggtggatttggtcagcattaagctgtattaagtcgagctgtttctacaaagactacattttgggataaacatggtaccaaagccacactaagacaatatagctcttggaagaaggggcttcatgccaccctcagcacctagtgcagagaactgttctcctaacgcacccacatacagacttcctgaggagaaattcctgcagcatccccaccctctgttcctgccgggaacaagtcccttag

SEQ ID NO: 127 - DNA, 599 bp, Homo sapiens
cttgccttctttaagctgcagaggaattttgatggaactttttcatctgttgaatgggagtggaatgctgtcctgcctatcacaaacaggaccaaatcatgagttattaaaaggcaacgatgagtatgtgcttacctctctcaaatccattcctcccatctgcctgccccgataccacataccagttctcattcacttcagctaggactgtgctcactgaggtcatctacataacctcctgcccatcctggtcaaggagccatctcccttagagcaggaaaggactttagtctagaaatkatttttaggttcaaggggaagtagaatgatatatcagaaataatatgaacctggatttaaatctcagtttattcattcattcacccaacattcttatttagttatttatttatttaagacaggatcttgctctgttgtccaggctggaatgcagtggtgcgatcatggctcattgcagcctcaaactcttgggctcaagccatcctctcacctcagcctcctgagtagctagaactacagtcatgtgccaccacacatagttaattttttttttcttttgtagagaaagagtttcacta

SEQ ID NO: 128 - DNA, 599 bp, Homo sapiens
ttttcatagagacaggatttcaccatgttttccaggctggtcttgaactcctgggctcaagcgagcctccccgcaccggcctttgggagccgacaaggaatccctgtcttctagtagtcttcaaagccagatccagcttgtctaggttaaggattcgatgcctccaatgaggcagagaaaatgaacatgcccgttcccccaaggccttcaagagcctccaggctcacagagattaacttgtcctcacccgcctgcctgccttttgtgcagatctctgcattgtcctgaagaaagactccrgcactcgggctgaaactccgtgtgccgatgaccctggccttgtcccgggaggcactggaagggatggcgcccgtcgcgctcctgaaggcccggagccacccgctgtgagcagataaccggcagcactgggcagcgcggaggcggccccacgaggactcagccccgggctgggagtcagggattcgccctgaagccttgcaggtgcccccgtcaccgggcgtcggattcccgcgggacgcggttttccccacggctgggcctccctttgggcgagtccccagcacaatgcgcgtcatccc

SEQ ID NO: 129 - DNA, 599 bp, Homo sapiens
taggtcatcagggacaaatcatttttaatggttggaaaattatttttattgtagtaggaactactagcacataaatctcattttatttcacagatacagcttagcaaaaatttaataagcaagttgatataagaaaaacgttgaatcagtaatactacaagttgtgaatcaggttcactgtgtactggttaccaacttgtctgagtgggatgagatagaacgcccacacacaacaagttacatgaagtgggttcactgcttacagagatgcagcaagggactgtagaagcgtaggacgcmtttgaggcctgagccctaaggctccggaaagctgtccaggggaaaggaatctcgactgtgtgtctcccacttgtacggcagcggaggaccctggaaagcagcctgtcctgggttttataccccggaaaacaagactcgctggactaaagcgttgaaagacattctgttctggggaggactggcacagagccaggctgttccagccagccactccctatctcaggatgttgcccttccagcatattctacagttattcttgagaactacaaggcagaaagggggtagaactggatgagtc

SEQ ID NO: 130 - DNA, 599 bp, Homo sapiens
tgacagagctaggatgggaacccaggagtcagaaatcggaacttgtgttcctaatcacttaagctatatcacaaatgagagaggtggatgattctatgactgtagggctctaaaatgatgtgaaggaatgaaatgtcttcacatagacaagaaatgtctatgtcctaattcccagatcctgtgaacattactttatatggtgaacaacgtgattcagttaaagatcttgagataaggaacttatcctgtattacctgggtaggacctgcatcaatcacatgtattttttaaaagagggasgtttgacacagacagaagacacatagaagagaaggtgatatgaagacagaggcagagattggagtgatgcagccgcaagcaacggaatactggcagcaaccagaatctggaagaggcaaggaatggattctcctgctggagcctccggaggaagtacagccctgctggcaccttgatttgggacttccgtcctccagaaaagagagaatacatttctgtagtttcaagccaccaagtttgtggtaaagttttttacagcatccacaggaaactaataccagtggtaaagtgtactttac

SEQ ID NO: 131 - DNA, 599 bp, Homo sapiens
cctactttcaagtaggtttcttccttctacttcctctgagtttatttttttctttctctatttttagtatcttgaattgaaactttagttggtcccactctttattttttaaatttttctcttttaatagtgaggacacttaaagctatgaatttccctctgagtattgctttggcaatatcccatagatcggatatccattcattcaacaaatttttttctagctttattgaagtatgattgagaaataaaaattatatatattttgggtatataacatgatgttttgatatatatacrcattggcaaatgattactacagtaatgctaatagcatattcattaccttacatagttactgtgtgtgtgtgtgtgtatgttgtgtgcatgtgagataaggcacatgaggcctactcaacaaatttcaagtatatcatacattaatattaactatagtcatgatgcttacattaggtctcccgtacttattcttcttataacagatttgtaaacttcgaccaatatctcccctcactccccaggccctggtaaccactgttctaccctcttgtactataagttcaccttttttttttttt

SEQ ID NO: 132 - DNA, 599 bp, Homo sapiens
ttcctctgctgggaacgctgcctaagctctgcaaacagcagctgaggatggtggatttggtcagcattaagctgtattaagtcgagctgtttctacaaagactacattttgggataaacatggtaccaaagccacactaagacaatatagctcttggaagaaggggcttcatgccaccctcagcacctagtgcagagaactgttctcctaacgcacccacatacagacttcctgaggagaaattcctgcagcatccccaccctctgttcctgccgggaacaagtcccttagcccagggaraggcatatgacccaagctgagccaatcagaggagccatcctcctggctactgtgattggtccagtgtggtcatgtgacatagcctggcaaattagagtccttttcccgggatgttttttagctggtgggaaagaatcctctcctgataggtcatggaatggtgaactgagaggttttggaagtcatgtcctcaccccataggaaacctctgtggcagggcagaaggaagccaacagttagagatgagaagctgagaagagagagtcttgctgatgtctaagtccctgtccccgactgcc

SEQ ID NO: 133 - DNA, 599 bp, Homo sapiens
gagcctagtttttcacaaatgttgattgatattttcatttaccctaacaggtaagaaaaaatgacatataatgaagacatataatggaacttcactcatttatcagtgacacagatattactttcattgaatcagaattcaatcagatattagttttaaatagaatatttcctcatttgtgggtgctattcacaatatataaaagctatagacatgacagactttgaaatttaatctgcattattaatattttcttcacccctttcttaagttcagagtctaaactttgcacaatcaacraaacattaactggagtcctgatgtgtagcatttgcccatttccatagtgtaaatactcccaccgtggccagtttcagctaccaacatcatgtccctggatacagagttgggaagagatgtatagtagcacagagctttgtagtattttcaccatacaaatgcaatagttgcaaacaatctcaagagcatagataatagtaaaacatagtgaaatagttcagatgtgacaagtgttgggtattacctttgtttcaaatattatttatcaaattgtaaatttgtcaaatttatttttaata

SEQ ID NO: 134 - DNA, 599 bp, Homo sapiens
aagatttcccaacaggcaaatgtcagagcctggatcagacttcagactcaggccccctctttttctccagtgccgtgtcacctctcactaaggggtcatctaatccagcctgctatctgatatactaatgccttcaacaacgtccctgtccagtgactatccagcttctgcctaaacactccccaagacacgagcctcactaccattccctgaagcagcgagatgcatctttagacagctctgccagaaaacacttgctcagtcagggtcaaaatcagccgtcttgtaacactcctgtcygagacctcagtaatcatgccatgcagccattcaaatgccttctgcgacacagatgccggcccacttccagcatctgctcgctcacctccagtttggggaattcaacaccactcaagccgaccctttccttctttgagtatttaaaaatcatcctccctcccgcaactcctccccactggctttgtcctctgtggcccccacagaccacatctgttccttctgccccagggtagcccctcagagacaacgaccatgcccctaagttttatttttcttctggctgcagccttaacaggtca

SEQ ID NO: 135 - DNA, 599 bp, Homo sapiens
ttcgggctgaggcactgtagggaccttgggttcaagctctataccctcccttagcaacagtcaattgcctgagtatagaataaactttgcctctgacccctcagctctagccccaagtctgaggtagttgagggtttaggagaaaaattaagctttctgttcaggacacaatggccaactggaaggggtccctgtctccaagtcagtctgggccaaaggatcctctagtaaaagcctaaggtcctcctcagtgccctaaggaaccctcccctgccttgaacacatacgagcttgactttstgtgtgtgctcagcctgttcctgagggtagacatgacaagatagctgggggaatatgtccctgatgaaaaacagttcagacaggggcatgtaccatgaaggatggggaaggggcccagattgggaaagcatgttcccaggatcacattgcaccacatggggagaaccaggattttcatctcattgcttgtataagttcccaatagaaataaataggaatattgtgagtgtgtgtggagcctccatagagccatagaaggggtgtgtgacttgcccaagatatgacagttacaagtgtcc

SEQ ID NO: 136 - DNA, 599 bp, Homo sapiens
taaattaatttttgtatatggtatgaggtaggagttcaaattcattcttttgcatgtggatatccattgtcatggcaccatttgttgaggagactgtttttcccatggaatgctcttggtacccttgtcaaaaattaattagtcataggtgtatgggtttatttctggactctctattctattccattgatctatatatctatccttatgccagtgtcacttcagtaaagttttctagttttcttaaaaaaaaaaaaacaaaaaacagcagattcaacccactttcttctggcctttaaktccaatattataattagaataaactgctcccaaaaggatgtgaataacacaccctacaacctggactgaggttttttcattattacactcatgacttctgctccaatatgtgtttgaacactgtttgagtgagaggcggtgtttcttaacattctcttcttctgcatatccagaacaaataatacaaagggaatctagaaactacctaaccccatagccagatcattctgacaatgctatagaaaaattaggtaggatatgccatattataaagtagggccatgagttcaaaaaacaaa

SEQ ID NO: 137 - DNA, 599 bp, Homo sapiens
acatttgcatattaaagggctaaggtgggaaggccaggttgttcatgggctacgtgaatgacacacctggtcaaaccaatcccctgagacctatgcaaaccagacactgcctcctccagcctccctatataatggactgcctttgtgctgcacacagggtttctctttgttccaagtcctcctcccttgtctttgtatgagggagctggtctcttttttcttccttctttcttgcctattaaactttttgctccttaaaaccaaaaaaagaaaaaatactttttggctagttcagcacasgcaccaaccagtcggagtggacacccagccaatcggaacaaatgccagctgtcaacaacaggaaacactgtactgtacctgacttagtgcaaaggggcctgggaaatggagttgctggctgagcaattgttgtgcagtgatgactcaccctgtggcaccagatgcacccatcctggtggacggctagctggccctgcctcaaatcacataaggtaccacaagcttccatctcaggtgtctgctttcaacccctgcatgtcccctccaccgcaccctatcatcaaggccagctagtccac

SEQ ID NO: 138 - DNA, 599 bp, Homo sapiens
ggagcgacatggtcattaaccacatatttgcacagcttattcatgttagttgggctgctccatctcactgagtctaattggacacgcccacaatgctgcacaacacatttacatcgacctggctgtcagcagccaataaaggactacaggatgtgtttggttccaaagggacttggctctcggcagaagcttgtttgcaggcacactgggtgtcttggctttctggcactgtggactacagcttaactcagagcatacattcaatcatttctcaaacatttgttagctcaagcactgtaycaggcacagcactggccctagcagggtctacaaagatgaaccaggcaccatttctgccacctcgaggaggtttaccttctgccctccatgatgtgaccctgtctagcaggccccctcctgcccacctggggcatgtgtgccatctaccagcccactccacattctctgcgcaggtctctgtgctcttccttcagcctggaattcttctgtccacctggctctgtctgaagacgccacctcctctgtgaggcctctcctgatttctgagaccacacaaatctctccctgggctgcgtttt

SEQ ID NO: 139 - DNA, 599 bp, Homo sapiens
cccaaggccttcaagagcctccaggctcacagagattaacttgtcctcacccgcctgcctgccttttgtgcagatctctgcattgtcctgaagaaagactccggcactcgggctgaaactccgtgtgccgatgaccctggccttgtcccgggaggcactggaagggatggcgcccgtcgcgctcctgaaggcccggagccacccgctgtgagcagataaccggcagcactgggcagcgcggaggcggccccacgaggactcagccccgggctgggagtcagggattcgccctgaagcctkgcaggtgcccccgtcaccgggcgtcggattcccgcgggacgcggttttccccacggctgggcctccctttgggcgagtccccagcacaatgcgcgtcatcccgcagacctgcccggcagaggcgggcgggcaggagcgcgcttggcttcctgtctcgctgaatggcttgaatgggcgctgggcccgttcctaatcccctagcggctgactgtcccggcaggggcggaggagggcgggggggcttgcagccctggcgtctcctcgcaggctgcaggcagtggggcccgcctgttctagtt

SEQ ID NO: 140 - DNA, 599 bp, Homo sapiens
ctgaatcctattcctgtcccacagaatacaaatgtttcactgaacacagtttgtgaagtgctgccctgaaaaatatcagctcttgtcctgacaacctgagagcacaggatggaggccgtgaaggaagcctggagacctctgtgcagagcctctttgttgtacatgtgaggttctgagacccagagagtagcaggtgttctcaggttgcacagccaaggaggggaggcaccatggccaagaacggccttttcactccaggcaggctggcagggctggcattttggaaagctctcagcagayagaattcacacaccttcctgtagaggccgcacaggattcaagagatggaaactgctggaaggagcactgtcttattgaaagtaatctcaagatcccaggaccacaaagctagatgtttcacatcagtctaattcccccaacacacaggcctagagaggagaaggacttattcaaggccacaaagcaagttcaaagcacagctggaactagaatccaagtgtcctgccccctgcctccagctccaaggctctgtctggcctttcctgcagttgttgatatgtctgtctgtcttctcagcc

SEQ ID NO: 141 - DNA, 599 bp, Homo sapiens
ggacatgctccaatgacacccatggctgcttcctcatagggcaccctctgcctctctggagcctgccgctggctctatcgaaaggggccagtgggaaatagtcatggggtcctactctgatctgagcagccagagggagtgacatgagctgagagttgtgaataaacccctgtactttgtacttacttactcaacttgacaaacattccccatggacttgccctctgtcagactgcactgggaactggggattctgtagtgaatcaggcactatgtttgctcttcagaatctagtaagakttgggctcacatagccaaaatgcaaagcagagcatggcagcgccctggaagatgaattccagattcctgctgcctggtccagaatcagcatagcccaacaatgctaggtgaccttggtcaagttgcttaatctctctaccctcagtttccatccttacccttctttctcaaagagctactctaagaagaaaataagacaattggcctggacatactttggagtcaaagctgaatagggagagacgattgctagtgtttcatagtgtcatcagtaacatagacaccatttattcagtatc

SEQ ID NO: 142 - DNA, 599 bp, Homo sapiens
atttatatgggcagattgaatgagcattatctccgcaaggtcttcccagtactttatagtttgcaaagcaaactttaatgtctctgaactctaaggatactctggcaagcaaagatagtctctcattttactaattaggaaactgaggccagagagggaaaggcacctgctctgtgcatcatgcgtgcatgcacatgtgcctgtgtatgtgtgtttctgtgtgcaagaaaggccttgaatgctcctgcagacctggatcccagttgtgagagaagttgtcagttctgatgcttggcgggyagcgttccggcccactgcccatgtatggagtcactcagacttagctaagtacacagcagccaggagcctgtgtttcagcagctaaaatcagtgggcagcacaaaaatgtaggtgtagctccctcgccgcttccaccttgcccctcttctctcccctcccacagggtggaactgcctggaataagctcagcccagataacaagtctacacatgcagcatccagggaataagcacagggcagctctggggcacacatggagacatgtatcttcatacacacgcacatatgtacacacaaat

SEQ ID NO: 143 - DNA, 599 bp, Homo sapiens
atggaaacttgcttatgatgtgttctgctagaagctgcaagacactaaattgcatgtacaggccaaatacaactgcccacagccatatgcctatgaaaaagatactagaaaggatatcagggtgttttccttctttcatcccctccatcctttgcatttacagatgaggaaactgagaccaatgggctgttcctgctgttgagaacctgtccctacagtttccgtgtggcccacaggggcagagtggaggagctggtgaccatgcaacaggaggatcaggtcatgaaagacacctggagmtggggtggagacagctagttcaacacgtggcttcagcacacacccactgagcaaaggagcctactcaatgccaggccctgagggagagaggcagacccgacccagtgctcactatcacggggcacatgggaggaagagacgatctctgcctgaggtgatcagagagggcattaagcaggagtggcatttggatctcaaagcagggtgaggacatgtggcacactggagccagtttgtaccagcttttggaggctagctgcttttaggaatttcacatgccagtgcttagacataggtat

SEQ ID NO: 144 - DNA, 599 bp, Homo sapiens
taataattaattaatttaattaattatggcaaattatactgggtttccatccaaagtttctttttagagtaaacgtacattttttaaaaatgagttagtttaaagaaaaatattgattactctcacaagtggtttgtagatatgacaaaaattgtaaaggcagaatcaaaagactaaaattatggtgaacgtccctggaacaatgaaaagaacatgggctttggaatcaggccagggtttgaatccagttcctccaattgttagcagtgtgacctcattactcaagctctctgagattgyttcctcctcattactttttaatggagatcattaaccaaacctcatgttggtgggggtgatggcagtgtgaagggatggagtgggaggaggtgcatgggggtgtgagggggggggcggtggggaggtgtgaggattatggcttactttcaccactgctgttcttacattcctctttcttattgttgcccctgggtcttgacccagtttgtaaattggggtccaatccaacccaattcccagatgcctggccaggctccggggacagaacccaataggaccagcttcctgccctccaggag

SEQ ID NO: 145 - DNA, 599 bp, Homo sapiens
ttgctctgtcacccaggctggagtgcagtggcatgatctcggctcactgcaagctccgcctcccgggttcacgccattctcctgcctcagcctcccgagtagctgggactacagtcgcctgccaccatgcccggctaagtttttgtattttttttagtagagacggagtttcactgtgttagctaagatagcctcgatctcctgcccttgagaccagcccgcttccgcctcccaaagtgctgggattacaggcgtgagccactgcgcccggccaactgctagtggattttacaacaaacrgcagcactgaagttaaaggtgtatggaatttacagtagagtattgcatgttactatttttaaattgtgggctacacattctttatattaggaaaatttgtaattaacaaatacatatattaacttgtatacttatattaacttatatacatatatatgcatacatttttgagaatccattgtaggaactcagataggagaagacagggcaggagaaggtagaggaagatacagtagggattataatccctactgtatagatacagtgtcttctatagcagagtgacactgtaggagatg

SEQ ID NO: 146 - DNA, 599 bp, Homo sapiens
tatctaaacaacatttgggtgttctaaagtaaccaccaggtaacacaatggtttgctcctatactacctatgaaaggaatgacgaaaggaataattaggtttgtatagaatattgtgcattatagtcataactgctgtgagaactcccaaatcagggtggtgtgcaggttagggcagagttttgggagaggcgaactgttctgtacaaaaaggagaagaatcataatgctgaggggaggggagggacctgggagaggggagtgagcagcaagcagggtggggcaccacctgaatgaaggwcagaaggtgagagcccttaggggcacagcagcacaaaagactttgggagaaaaggaattaataagagggaatttttaagttgtttgtcatggtgtgtgctatgggcctccttccctcttgtcactcttacactgtctttagaaaagtcattctttttttgtgaaaggggattctccagggagtattgaaggatgcagggatgaattgtttgtttctgaaatggagtcttgctctgtcactcaggctggagtgcagtggtgtgatctcagctcactgcaaccttcacctcttgggttcaa

SEQ ID NO: 147 - DNA, 599 bp, Homo sapiens
gcaaagcgtgccattgaggctgtgctgtcagcggatcctcggtctgtgtaccgccggaagctttgccaggaccgccttttctactttactgtagacatagcgcatgtcacttgctggtttggtgatggctttgcagaggtgctgaggatcaagccggcttctgagcctgttcatatgactggccctgtggggtccttggtgtctctagggtcttaaggagcctccctcatgtctttaaggtagcatcattgatctttggatgtggcttttggattttctgaacaagctaatgttgtgtcragaagcaacactttgtgatctcatggctttgattgatttgggctgttcaaaatgtttatttgaaaaacgtatacattaataaacttaacaaagagatataaaatacagagaaatcaccaaatgctttttgatctgttgatattaaagaatcaaaaggtgatgctgtctcgataatttctgaaagaagctgttcttggtgcacttgataaaattagacaaagttcattccagcagagactttctagttgaggggatctattcagtaattcatattggttttcctgtctgtctggaaaagc

SEQ ID NO: 148 - DNA, 599 bp, Homo sapiens
agtggactgaggagggctgagcggaaggatatgaggtcaaagaggcactagaaaagggacgaggtcacctaagggttttataggccattgtaaggacttgagttttgatcttgggagaagtaaaaggcagaggaacttgatgtccgtatccccagagagcaggggaccatctctggggtcagatcactacctgtgtgatttctgggatgtcacacctcagtgacatgcaaaggcaagctggagttccttattgagacaatgaagactttcattctatatccagaaaataaaggcttacamaaccaaagatactagattttcccctgctttactagcagggagagaatgaagaggctaggggggtatttggctttgataatccaacctgaaggaggtttttctgatgggaaaaagaagtctaaaagttgggactggagatacatgccaactgactctgagatttccccatttcttcctgtcagagccacagccagcccacactgttgccttttgcaaaggaaaagccaccccttcctctgggtggaggcagccacctgccagggcccaggaactggttttagccccaacatgcaaacttc

SEQ ID NO: 149 - DNA, 599 bp, Homo sapiens
acctcagccctccttcctttcttgtctttcagcatcaatgcagagaaaagaggagcagcaaagagggtgcatttccaggttagtgtcttagtccgtttgggctgcaataacagaataccataggcccatagactaggtggcttaaattgatctctcacactgctgaagggtgcaaatgccaaaatcagggcactggcagattcagtatccggtgacgcccactatgtcctcacatggtaaaacagacaatggggttctcaggggtctcttttattaggccactaattccactcatgaagsctcctcccccacaatctactcacctcccaaaagcctcgcttcctaatactgttggaccggggattaggatttcaacacatgaactagggggatataaacattcagtcaatagtagaaggttctgcctgttcagcccagaactctcaaggtctaaacttaatctctcctttcccatgaggaccccggtccacagcctctgaggctgtgtcagcttctagagcccattactctcagaagataaagcccccagcagggacactcataaaaaggaatccgaatctggagtgcttaggagattg

SEQ ID NO: 150 - DNA, 599 bp, Homo sapiens
caaatatttaacaggaggtcctctgaggctgtataaatatccagtttcttcttaaaattttgtccaccaattttatcatagtcagtggatattgcaattactgttatagtgttctaatggtgatcttctgttttcttagtttcttctatatttactatttgaaattcttctataaggaaaatttatctcttcttccccgtttatttatttattcaatccatttttatttatatcaatatggactcatggatatttattttattcttcaggttataatgcactaccacatttattaacctygttgctcaactttagccactgggagctctttcaggttgacttttacgtccttttgtcatgccctatctttattttttcaatatactttcttacttgctggcactcctttaggatttaggaccatcttgtatttattttccttgactcagctctagaactacccatttctccaaggggccctggttccttttattggagaatggtatttaggaaccaagatttgggggctgagagtgcccattgctactggggtgtcactgcttctaggccttctcagcagacagagcaaggaaatatat

SEQ ID NO: 151 - DNA, 599 bp, Homo sapiens
cattctgctgtttccatctctgctccatgtgctacccctttggctaagggggaaatatgtttttaaatctcaaatagtcaggctcattttctgcttcacacagtccttgttctctcttgaggaacttggaacatgctcctgagctacactggaaaatatcgaagtgcccttctccataattagccagcatccctgccatctggaccctctcatacctcacagggatttccttgtccacatggaactgagactgatccaagagtccagctcctgctgactgaactctgccatctgttccgmagcttgccttacctgctcctgtccaaccaggcacccctctgtgggcctgtgctttagtcctttttcactctagaccctggtactactgaggtggcttgcgtggtcctaaatttgctagtctcttattttgataaaggctgcaagtagcaaatatccttatccttgaaaataagggcatctaggcccctggcttctttttatgtttatttatgcatgtactatagaaaacttggaaaccatacttacagagaagaaaataagaatttccatccaaggcttttttaaagggccaagcactg

SEQ ID NO: 152 - DNA, 599 bp, Homo sapiens
tcgatcaaagtaatgagaatgaattacagtgagcccttaatgacaagagaaatctagcttcttagtagttatatgaatttcagcaaacctacttggctttgctgagtctgtttcctaaatactaaaaaaaggtggattatcgtcctgcccggggagctgttgctaggattaactgagaagcagtgtacacaaaagtcccatgcatagcacttggcataaggatggtgcttaataaatattagacctgactcttggtagtcatagctcttccctgctgcaccaggaaggcatagcacctcyagctggttcagaaagcagaaaggaacctgggcaggggcccgttactgggcctagagcttcctgtacacctgacacattcccctcctctgtttttaccatgtggagactaacaggatctcctcccttaacccattttcacctatttgcctctccctctccccttgtctcaataatctcagctgcctagatggtattttttagtacatgtgacactattttcaacataacatacatttgcaaagcatctgctatgttctaaagatggaccccaggtcctcctctccattcctatcatccag

SEQ ID NO: 153 - DNA, 599 bp, Homo sapiens
gaggggcctggggtctctgtgtttaagacagcactccccagcgccagccctggcagaggaagagtggaggtatggggagggaggcctcaaacagaatctagagatcatattctagccccaacctggccactgaatggtatttgctcccctctgtcacttcccctctgagcctccagttcctcaactgaaactccgaggcccaagctaagtgagcctgcaggttgtttcagctgtacaaatctgcatgtggttcaccgtgagcctaaccagcaataggaatactaattgtatttcatgccsttactcccagttaccctctgaaggagtaggtaagggtttgggctagtagaaaatgtcagagctatgtttaggctgatggaattgaaaaggatagaccagtagcatggggccagatgagtgctgcactttcagtgaaggaggtgtcttccagaaattgccacaggtatatacataagttggccagactagtcagtcatctagtagcattacagatgcaggttagtacatacatctgccattgatggccatttactgagaggaatttagtcttcaacttccctgaaaaataacctataatc

SEQ ID NO: 154 - DNA, 599 bp, Homo sapiens
tggtagaataaaagaataaaataaaacaaaataaaaataaaaaaataaaagaataactaaggaaaagtggttaacaccagggaagctctctacagaaaatagggactttggaatgtaattgccacagagttgaatattcctccttagaacagagcttggtgggggatttaagttggttcacttttctgaaggattagtatgagagatccccatgggaaatcaagtgaatccaagattaatctgctttctgaatgtaaagactttggatggaaaattaggctgcccgtgtgtgtgtgtgtrtgtgtgtgtgtagactgtcttggggtcaattgctgggatgggtagggacatatttcaaaatgagcagggaccacagagtgaggaaacatgttcaaattctggctctgccattaccttcctgtgccatctggcaggtggggccagtaatcaatcatctttgggtttccagtttcctgattccaagatgagggggtagaatccgaagatctccaaggccttgatgagtgctaccagtctgcaaattcccaagccaagttcttcaatgaatcaaatttcctccagtagccatttctaccaaa

SEQ ID NO: 155 - DNA, 599 bp, Homo sapiens
tgtttttatttctcccctggttccctggggctttacttcttaatgtggtcactttgaacatcattggcatcaattcacaacagggaaacatctgtgagaagaaacacaccaagcggggaatctttctggaacaaggatgcagcaacacagaaattagcctgatcctgttgccttaagctgcattcatgtagtaatccaggaaatgcagtctcttttttctgtgattggccagacaaatcctccttctgaagaaacttagatttcagcctccccgaaccaaaagaaaaaaaagaattgggyaaaaacatcatagtggagcatctatcaacaaaatatgtgcacggagaggtttcaaaatcatcttgaagactcacaagaattgtctttccaaaagcagtaaggagtagaagtgtacaactgattcaactaaggacagggccatcagctctggatgccaaaatcttcaccactcacagaccagcaagggcaggtcttcccactcggcatccctagatcctgaatatggcccagaggtctttagcttcccgttgcagctttttggaagccggactctctcctctggctgtccctgatcctcc

SEQ ID NO: 156 - DNA, 599 bp, Homo sapiens
ttagggtaaataataccagaaaagcaacaggtacaacccaaatcaatcaagatgtgtgccagcaccttcagttataaacactttcttctttttgccccttaatttcagagctatgaaaatttccttccttagaagaaatagttcccaagacagccccactgtaatatgccagaagaaatgtcaaaatcgctactgtggccaaatcttgtcaaaatggacacactacaaccatcataccatgttggttcttttgtaaagcaaaggtgacaggtgacaagcactaagaggcacactgagaaraagtatgggctttggtttgtgctgcagcagtttgccaagcatgtgaacatgggcaagctacttaaggtttctgagttctaaatctctcttgtggaaattgtgcattgtagtatctacattacagtactgtactgtagactggaaagaaatattaatatatagtgtctggcacaaactagggttcaatgaatggtaatattattaatattttaattgaatgctgtattttaaatttaacattaaaattttatttaaaaattagctgtgacagattatattttccaaaaatggctgcagca

SEQ ID NO: 157 - DNA, 599 bp, Homo sapiens
agcctagcaggttgcctactggttgttattttgattttttttattagatttgattaagccaggccaggcctttcctgggagcaggaaggttctggtggaagacacagagaggactctggctgggtcctttccctcaggagggccagccaagtcagagacagacgccatcatagagaaagctgacatgccatcccgggggcacactgaagtgaagggcactgaggagggggcgatgatttcaaaacagagggcttcctgggagctagcctctcagcaggtctcagcaaggcaggggcgacrgggtaggtattcctggctcggagcatcattttaggcagcagggcccaccgatcagtatgtcctatcagctgctggtgtctttttccctccatgacgcatcgtttccaccaactgctaattatctcatttccttcagaggctctgattgtcatctacaccccctggctgagttcctctgaaacgtgctaggggccacctaagctcctcttctccagtttgccctggtcacttgcccagtgggtggtttcccaggcttccctcttctgctctgtagtgctcagatgaagaaaaccatgtct

SEQ ID NO: 158 - DNA, 599 bp, Homo sapiens
ctctgaatcaccaagcgagacctattttaaaggtgaccaggtcgtgagatatgatctcagcacggacccttctcccggatgacaagagagcaggtagttaggcgggtactccgtgtccgcgcatacccagactccgcccagcgcccgggcaggccaccccgagccccttaactgcgcaggcgctctcactcagaaaggccgctgggtgcggggagcgcagaggcggtgcagggcggctggctcgcctcggcgtgcagtgcgcgtgcgtggagctgggagctaggtcctcggagtgggccrgagatggcggcggccgacggggctttgccggaggcggcggctttagagcaacccgcggagctgcctgcctcggtgcgggcgagtatcgagcggaagcggcagcgggcactgatgctgcgccaggcccggctggctgcccggccctactcggcgacggcggctgcggctactggaggtttgggccgcgtccgcgctttccccttccctctccccgcctccccggtccccagactggctcgtgcaagccgagtcccggggcccgggggtcgcgtcaactccgctggcgtatgtgtgcagat

SEQ ID NO: 159 - DNA, 599 bp, Homo sapiens
atgagtcctctacgtcagcactcctcgccatgcgccgttcttccgtgagcatcttaacaggtctctgctggctcttcagatttcctgttgtttttccacacctagggagtgtgcaaggaggtgttccagctcaagggtggcatccacaagtacctggaagagtttcctgatggcttttacaaagggaagttgtttgtttttgatgaacgctatgctctgtcctacaacagtgatgtggtgtcaggtaggtcagcacaggctcagagcccaaactgaaatgaagcacattgtcagttcacyattctagaaaaatgacacagggaagacaggccagtgctcattactgagcactgaataagcagggaaaataagtacattgtgccaccattttcccagctgtggagctgagagaaccctagcccaggagtcaggaggcctgggttgggatcctggcttcaccattgctagctggacaagcccattaacatggggatcatctcacctgccctgcctgcctgtctacctgccaagagctgtactactgggctaattcagggctcttaacctggaattggtacatagatttcagggattctgtg

SEQ ID NO: 160 - DNA, 599 bp, Homo sapiens
gcatacattgtttcattgtgcttagcagaaattgcttcttttttttttttttaactgaaggtttgtggcaaccctgcaacaagcaagtctactggcaccatttttccaatggcatctgcttgcttcttgtctctgtgtcacatactggtaatttttacaataaaacaagcttttccattattaattgctgcaatcccataataaaactttaaaaaataagaagatgctttctatgtatgagcaaagaaagtgtttttttgagatggaatcaactccttgtgaacactgctgaatgacaaygaaggatttagaatatcatagaaacttagctaataaagcagtggcagggtttgggaagattaattccaactctgaaagaagttctactgtgggtaaaatgctgtatcaaacagcaagcatcacatgcttcagagaaatctattgtgagaggaaaagtcaatctaggcaacaaattttattgttgtctatttttgaactccctgacccaaatagtgaaggatgtatctacatatgtataggtacatatgtatttacagacatacaagtacatacatgtaatacacacacacaacacactt

SEQ ID NO: 161 - DNA, 599 bp, Homo sapiens
acctgacaccacatcactgttgtaggacagagcatagcgttcatcaaaaacaaacaacttccctttgtaaaagccatcaggaaactcttccaggtacttgtggatgccacccttgagctggaacacctccttgcacactccctaggtgtggaaaaacaacaggaaatctgaagagccagcagagacctgttaagatgctcacggaagaacggcgcatggcgaggagtgctgacgtagaggactcatggccgctgctatcagtcaggggctcctggctcccactgcgggacactgaggaaytgctgaccaaggggtcaaggcaccaactggccagatcttgcctcctagggggatgggtgatgacagcaggcagggccttcaccctctagatcctgggaagcccaactatttttgctgccccctaaatataggaagacacctgttgggacatgtaatgttactgaatttgctcttctcaggatcaagcaaaaggatcaggaaaagggagatgttaactaatctctcccttttacatctgatgagtctctatgactgttaagtgtttttcatatcttgagattaaagaccctcagattttg

SEQ ID NO: 162 - DNA, 599 bp, Homo sapiens
atgcttgtggagttaaataaattgaatgaataatactgagtaagactacttgcatatctctttagcactgacaactcatagtccaaagctgcaccctacaaccgccttgtctctgcttgtgtgactttggtgatggggaacactccacttgagaacaaacaacccattctattgttgtctagcattggccattatttattgtaataataacaataacaatagcaaacacttatatggtacttactataatactaggcagtttttaagcatattcaccagtctcagtttagagatgagacractaaagcacagagatgctaagcaacttgttcaagatcatacagctaataagtgacagagctgggttcaggcaggcacagcccagccccagcattaatagtctcaaccaccaaattataagtctctaaactggaacaagtctccctcttaccctcttacttcctccaccagagattgtcctgcatgggttagcacagagctggaatcagaccaggttcttgccagtaccctacgtggctaaccaggtggcttgaaacagagagggcatctctgcctcaagcttagtcttggagttattg

SEQ ID NO: 163 - DNA, 599 bp, Homo sapiens
attaacggtattttataaaattttcaactacaaccactagtcaacctaaaaccttagtgcataaagaactgaaccacaaaagggtttcatgctgccagcattttgccttctggtttcccctgcagagcacaggtcatcaatagaacacatccagttcaaaggcatggactccaccgccacacactagacaccactccggcacttgggaatgatgggcccagtcaggtccgcaagcctcctcttcctcactggaaaggaatggagataagatataaggcacttttccctgtagaccagggrccagcactgttgctgagaaagcactgtgaaacctcgcactaattggtttgttaacagagtctgaagtctgttgttttgaaaggacagtatgtgtgagttgtacaacagccctccagtctgtcagtgttcattgatgagggtctgtggtagtggagattcattcatagatatctgctaagtggctgaccacacagtcccacgttataagtcggctattttctttgcttacagaagctccagttgaagatctttactccggtgttcagataagagttaaaagtttagtcagtcttcttaat

SEQ ID NO: 164 - DNA, 599 bp, Homo sapiens
tacccacattctgtagggttgttgtgaagatcaaagatgatggatgtaagatgtgtatctcagagcctggtccacagtacactctcaacacatgtaacatgtaagatgttgttattagcatcctcttagaaagtcagagctgaaagagttgccccgcagataaaatgattgacccacagaagttacagagaaacagtaactccataagaataaagacaaactagaattgcctagagagtaatgtgaatggtgaggaaattcaaaagccgcattgaatgggggtgaatgaagaaaactggraattttaacctggagaaggagagcttagtggtccatgagatctgtgcaacagatgtcctgagtgaagcagagaacagagctaagcccaacaaacaggacttacggggagctgacttctaccggatataaggaagaatgttctcattgcctaagaatggaagtgatgatgttggtaaggagtgtgtcccccatcatgagagggactcaatcagaactggaagagcacgtagtggaagcgatgaagaaggaaggtgagcgtggtagagggtgtgaactttagggaccttggaggtccctca

SEQ ID NO: 165 - DNA, 599 bp, Homo sapiens
actgtactatttcttataactgcttgtgaatctactatgatgtcaaaataaaacttttaattaaaaagaaaaaagcaaaaataacgatttgttgtttatctgaaagtcaaattaaactgggcatcctgtattttatcaggcatctctgggagcttgttggagatgcagattccctggccccacttcagacccactacatcagaatctgtgttgtaacactaaagtatgagaagctctgattagtggctcctgggtgtgttagtccattttgcattgctatataggaatatgtgaggctgrgtatttacaaagaaaagacgtttgtttggctcacagttctgcaggaggcaaaagaagcctggcaccagcatctgcttctggtaaggactgcaggggagcttccaatcatgcagacagtgaaggggggatcaggtgtgccgcaggccacggggaggtgtcccagactgttttcaacaatcagatctcaggtgaactcattatcacagggagggcatcaagccattcatgagctgcccccatgacccaaacacctcccatgaggccccacctccaacactggggatcacatttccacatga

SEQ ID NO: 166 - DNA, 599 bp, Homo sapiens
agctgtattcattcaaaagcatagtctggggtcaaaatcctacacattccaggagctcctgttattggtaatgactgcagatgctgagaatcaggctttgtgatctgtgctgtcgatctaagagggcagggacagctagtagaaaaatgtccaatagcactagtagttaaaacaaaaagcacataaaacaacaataaagtctatgattggcatatattttctgaaatgaaaaatctctatgctggtggtgatgacataaaactgaaaactacacaattgtttcaaaggaaaggtaaagcraattttgattacataaaatatgacgtcttctgcatccaatagtggaaaatgtggaaactatacaggaacatggaaacttgcttatgatgtgttctgctagaagctgcaagacactaaattgcatgtacaggccaaatacaactgcccacagccatatgcctatgaaaaagatactagaaaggatatcagggtgttttccttctttcatcccctccatcctttgcatttacagatgaggaaactgagaccaatgggctgttcctgctgttgagaacctgtccctacagtttccgtgtggc

SEQ ID NO: 167 - DNA, 599 bp, Homo sapiens
taagtatgaaatgacaaatgagagtgagcgtctgaacagaatcagtacagcgtgtgagggaggcacttggggtagggctgggggagaccagggaagagctatggagaagagcctaggagcagaagagtagcagtgagaaagtgcggagaaggtccagggcttgggagcggcaggaggagcggttccgggctggggagggcaggcccctctgaacggagtggcctggcaggcttgctatctgctgatgggaggagcctggagggcaagctcaggtgtgccctcctaactcaggtgtgacaygggtgagatgcatcactcatgtattcacctaactgctcactaagccagtgcacattattgaactcctactgtatactaggatgaacaggagccagtccttgcctttgggaggcccaggaggtgatgaggaggacagacgagaaacatgtattttttttttaaccttaaaatcttttatcacttcaacatgtagatttcaacattaaaagcgtccctgctgggcaacaagcagagtgcacaggttcctggcagggctaagttcttggcgcatagcctacagggttgtaggtcagaggctg

SEQ ID NO: 168 - DNA, 599 bp, Homo sapiens
gagctgggagctaggtcctcggagtgggccagagatggcggcggccgacggggctttgccggaggcggcggctttagagcaacccgcggagctgcctgcctcggtgcgggcgagtatcgagcggaagcggcagcgggcactgatgctgcgccaggcccggctggctgcccggccctactcggcgacggcggctgcggctactggaggtttgggccgcgtccgcgctttccccttccctctccccgcctccccggtccccagactggctcgtgcaagccgagtcccggggcccgggggtcscgtcaactccgctggcgtatgtgtgcagattctccccgagtcggagagggaatccgcccagccagccgccttgtcaaagcgtcctgtccacgaccacagagcgttcctctgtcgcacgcgggcctcctgacccccagccccgggccttcttcgctgcacctcggctgctggcagcttcgatttttcgtttagggatgcagccgccccggccgggaggtgtcagccactgccaggtgtcagggcctcagctgtcccggaaagaggagttagactagttcttgattctggcctctatacct

SEQ ID NO: 169 - DNA, 599 bp, Homo sapiens
gttccagttgttccacatcctcatttcacatttgacacagttggtctttctcactttagccattttaatgggtgaattgtagtatctcattgtggttggaaaaatactttaaatcaaaccaaaatacagaatgaaaatcttaccttatcagctcatcagggttaaagcagcttatatcagggagccaagcagacacgtcatggctatgtggaaggcactcttttaaagggttcttttcatctgcagacgaaagactctttgaggtgctcaatgtcacagccagttgctttaaaatagaakctgtctggtgataaatttcatcagcatgttgtgttgccacatgtctatggatgctggtttggtctgtgaatagctgccgacagcatttccacaatttttctttacattcaaaactcctttttgttggaacttctttggttttgacaaaaagggcaaaggcctgcaattagatttcaaatgctaacactgttaaataaccttaggtttatggctaatagaagtctcactaagattctttagattatctttccacaggggcttctagttttatgttccattatgttatacaattctctgag

SEQ ID NO: 170 - DNA, 599 bp, Homo sapiens
cttaaccaagaaacaaataatagcaatggtggtgcaccactgtaccccaggttctagtcatgtgttttttaggacgatttctgtctccacgatggtggaaacagtggggaactactgctggaaaaagccctaatagcagaaataaacattgagttgtacgagtctgatcatgttttctgtactcttggggcctctattgtgggacttaacattagtctagatagctttttaaatgctggattaaaatggaaaagagctgttttcatggttgactatctgattgttgatcaaagaaggcaytgatgtttattttaagtagtgctaacatttactgagcatttaccaaccaggtgccaggcatgcacctaagtgccttaggagtatgttcatttagtcccaggcttgcttccatgctgctttcagacttctaaggaaaggaaggtgaagtctcacatcaccgcaggcatctgcatcaactttagtgaaattttcctgtagcttgtcaaaagaattattgggtctatgcattattttttgacaaaaacaaattccagttagttaaaataacctgatattttcaaattgtttgacatgacatt

SEQ ID NO: 171 - DNA, 599 bp, Homo sapiens
tcatcctctactcctccctcatcccggatgacagggaatgtatccagacaaccacccaggcccatgcagacaccctccaccaacaagatgctgcccataaccttatagggaccctagctgtccccagaactgaccccaattgggattatgaagcagtttcctcagacagacagaaacgaagctatatgatatcatgcctcctagctggcatggataaagctgcccttaaggctgtaaactctgaaaagataaaagaaatcactcaaggccccaatgaaaaccctgctcttttcttctccsgcatttcagaggccataactaattataccaccttaagccctgataccaatgggggcagaatctacctacccatttacacttcatttcccagtcagcccccaacgtctgaaagaaacttaaaaaactagaagatggccctcaaacctcccaaagagacttaatcaaagtggcctttatggtctttaatgatgatggctagaatttaaaaaaagaacaaaagaaagaaaagaaaagaagtattggcaaagatgtaaaaaaaattggaaccctcatacattattggtgggaatgtaaactag

SEQ ID NO: 172 - DNA, 599 bp, Homo sapiens
gtacctactctatgggtaggttctcaatgactactgctgaaggaattcaaactgaaatggaagaagttttctcatggatcaaaagtcttagctgaagacttcatgttttccattaactgcagtcactcacatctatttaagcttctgctatcaaaaatggaaaagcagccagtgacttctgctttaaccgaggctgtcttgtattttttgtaaacttttttctatttttaaatttttatctagagaaagggtcttgctctgtcatccaggctggagtgcagtggcacgattgtagctcaytaacttcaaactcatgggctctaacaatcctcctgccctagcctctcaagtagctcagactacaagtgcaagcccagctaatttttaagattttttgtagagatggggtctcgttatgttgctatgtagctggtctcaaactcctggcctcaagcagtcctcctgctgccacctctcaaaatgttgggattacaggcagggcgcagtggctcatgcctgtaatcccagcactttgggaggcggaggcaggtagatcatttgaggtcaggagttcaagaccagcctggccaacatggtga

SEQ ID NO: 173 - DNA, 599 bp, Homo sapiens
gatcttccaactttagcaccatcttcacatttaatggttggcattttactgtaagcaggagccctctcctctctcctgtttcttatctacttattattgatctatttattcttgcacagactcatagattcctatttttcaatggtttattttctttattttcttaattatgttgatgctcaaattgtcctagatttggccagtggaagcctcttcaagttgatttccttgtccttttgacaggtccccatcacttttttgagcactgccttactttccagcacaaggtatttaatgctyattttgtaaaggaaaagtgtgtgattagaacaaaaaaaggggccctcacagatcaacaaagatggttgtgcattggcaattataggtataattaagtgcaaattattttgagcatataacacagaaagaaataactgcacagaaaaaagaaaggaccataataaaagactaaaagaaactcttggaattatgaaaagtttttttcatattgttctttttttctgtatattcaaaattttatgtaatgagaatgtattagttttataatgagaaaaattacataaaaggtttttcctttt

SEQ ID NO: 174 - DNA, 599 bp, Homo sapiens
gatcgtgaagaggtgttttcaggcatctttattggtgttttggacatatttatttatcctgttgtaatgtctcagagctcgtccactccattattcttaaaaaggaaaaaccttttatgtaatttttctcattataaaactaatacattctcattacataaaattttgaatatacagaaaaaaagaacaatatgaaaaaaacttttcataattccaagagtttcttttagtcttttattatggtcctttcttttttctgtgcagttatttctttctgtgttatatgctcaaaataatttrcacttaattatacctataattgccaatgcacaaccatctttgttgatctgtgagggcccctttttttgttctaatcacacacttttcctttacaaaataagcattaaataccttgtgctggaaagtaaggcagtgctcaaaaaagtgatggggacctgtcaaaaggacaaggaaatcaacttgaagaggcttccactggccaaatctaggacaatttgagcatcaacataattaagaaaataaagaaaataaaccattgaaaaataggaatctatgagtctgtgcaagaataaatagat

SEQ ID NO: 175 - DNA, 599 bp, Homo sapiens
aacaccaataaagatgcctgaaaacacctcttcacgatcatcagtcacactatcaaaaactatcagtcctatggctgaaccagggttggaaaatgtgggtttctatatcttattctatggtgtacctcttgggttactttgtctaatcacttcatcaatttgggtcttaacttcctcacctgaaaaagaggataataacttcttgctatattagcaatgtaatgagactcaaatgaaaccatgtatatgaaaactcctttaaaatgctacaaaagtaaatggttttataaaagagctttyttaaaaatggctgctttattgaaacgttttaacagtttcttttttaaaaaagcagccaatttggaggacctacgcatttggatggtgaagccctttggcagtctgagtaagcccatttgggttctgtgttgcagcctgggcaactacctctccatggctgcatcttaattggggtccccagaagaaaccctggaggaagattcatgtgaaagtgatgtattaggaagtgctcccagaaaaaatggtaaggcagtggggcaccgtatcacacaaggcactcggaggagaactttggctta

SEQ ID NO: 176 - DNA, 599 bp, Homo sapiens
ctgaccccagcaaaaagcaatatttaacagcctgacaggagaaatgcccaaaagagtataaatggcagctgtattcattcaaaagcatagtctggggtcaaaatcctacacattccaggagctcctgttattggtaatgactgcagatgctgagaatcaggctttgtgatctgtgctgtcgatctaagagggcagggacagctagtagaaaaatgtccaatagcactagtagttaaaacaaaaagcacataaaacaacaataaagtctatgattggcatatattttctgaaatgaaaaawctctatgctggtggtgatgacataaaactgaaaactacacaattgtttcaaaggaaaggtaaagcaaattttgattacataaaatatgacgtcttctgcatccaatagtggaaaatgtggaaactatacaggaacatggaaacttgcttatgatgtgttctgctagaagctgcaagacactaaattgcatgtacaggccaaatacaactgcccacagccatatgcctatgaaaaagatactagaaaggatatcagggtgttttccttctttcatcccctccatcctttgcatttacaga

SEQ ID NO: 177 - DNA, 599 bp, Homo sapiens
cttcacatcattttagagccctacagtcatagaatcatccacctctctcatttgtgatatagcttaagtgattaggaacacaagttccgatttctgactcctgggttcccatcctagctctgtcatttgctgtgggattttggggaaattactgaatatcgtagaatctcagtttttgttttttttttcatttgaaaactcaaggatagtatctaccttatagggttgaaatagggattaaacaaaatactctgtaaaaacacaaaatacaggagctctcaagatttgaagggcacttasagactatataatcccatctctctaacttagcactgatgagtctcaatctgattttaggtcatcagggacaaatcatttttaatggttggaaaattatttttattgtagtaggaactactagcacataaatctcattttatttcacagatacagcttagcaaaaatttaataagcaagttgatataagaaaaacgttgaatcagtaatactacaagttgtgaatcaggttcactgtgtactggttaccaacttgtctgagtgggatgagatagaacgcccacacacaacaagttacatga

SEQ ID NO: 178 - DNA, 599 bp, Homo sapiens
tactttatatggtgaacaacgtgattcagttaaagatcttgagataaggaacttatcctgtattacctgggtaggacctgcatcaatcacatgtattttttaaaagagggacgtttgacacagacagaagacacatagaagagaaggtgatatgaagacagaggcagagattggagtgatgcagccgcaagcaacggaatactggcagcaaccagaatctggaagaggcaaggaatggattctcctgctggagcctccggaggaagtacagccctgctggcaccttgatttgggacttcygtcctccagaaaagagagaatacatttctgtagtttcaagccaccaagtttgtggtaaagttttttacagcatccacaggaaactaataccagtggtaaagtgtactttacctccccgtttgtcatagttgctcttgatggctggaatggtctgtttggggctttgatatggtttggctctgtgtcctcacccaaatctcatctccaattgtaatccccacatgtcaaaggcgggatctgatgggagacgactggattgtggcggcggatttcccccttactattctcgtgacagtgag

SEQ ID NO: 179 - DNA, 599 bp, Homo sapiens
tgaagtaagtccaagttaggtctatacctccatgccctctcaccacttgcctgctcctttcctggccccaccctcaccagtcttgcaagtttcttcaccactcatgaaggaggggaacacctgtgagggtgtcacaagtcatgctaccaagaaccagaaccatacagggcttgactccatccctgagcccctgggtgacagtttcccatggctctgacaaggtgctagaagtgagctggcatatgctctgccttctcagacaggctactggtctggtctgtcctggtgtctgtcagacakgagcccagccttatgtcacccagttcctgaactgacgactccccaggatgatctatgtccctgatagcacaacctctagaccactgggtctgaggaccagagtggtggagccatgggatgctggagcaggaaagtcatgtagggactgggaacttgttctaaccctcccattgttggggggatgccttagacctgggaggacagggtcttgtccaaggtcacacagtgcaacaggagtggaatggggcatgaactcagggttttttcttttatttccagttcagcatttttgcctcatt

SEQ ID NO: 180 - DNA, 599 bp, Homo sapiens
ctacaggtgcccgccaccacgcccggctaatttttcatatttttagtagagacggggtttcaccgtgttagccaggatggtctcaatctcctgaccttgtgacctgcctgcctcggccttccaaagtgctgggattacaggcatgaaccatcgcacccagccctacctgatcaactctttagggtatggatataaaacaagattcatggcacaagccaatgccaactcattggaagtggtatataattttaaactttgaaagaacataaaagacacaaacatatgcatgtgtgtaatatratcagagaatattttaggaaaataaaaaccatcgataaccctacatgcctagataatccttactaacattttggtatacacaaccttccatgtttatacatgcacttctggttttgtcatgaacatgcattctatattctccaaatatctactgagggtctactacatgctaagctctgttccagcactgagaatacatcaattaagaccaaaattcctgtcctcatgaagcttataaatgaagattatactttcagatagtaataaattctttgaaaagcagaaagggatagaacgtg

SEQ ID NO: 181 - DNA, 599 bp, Homo sapiens
gtagtatctcattgtggttggaaaaatactttaaatcaaaccaaaatacagaatgaaaatcttaccttatcagctcatcagggttaaagcagcttatatcagggagccaagcagacacgtcatggctatgtggaaggcactcttttaaagggttcttttcatctgcagacgaaagactctttgaggtgctcaatgtcacagccagttgctttaaaatagaagctgtctggtgataaatttcatcagcatgttgtgttgccacatgtctatggatgctggtttggtctgtgaatagctgcygacagcatttccacaatttttctttacattcaaaactcctttttgttggaacttctttggttttgacaaaaagggcaaaggcctgcaattagatttcaaatgctaacactgttaaataaccttaggtttatggctaatagaagtctcactaagattctttagattatctttccacaggggcttctagttttatgttccattatgttatacaattctctgagaagtaacacttctcttattagtaagctttagaagagactattagagaaattaaaactaggctttatattgaggaggtt

SEQ ID NO: 182 - DNA, 599 bp, Homo sapiens
cttaactaaagaaaaaaatagaagtgaaaggatttgttattttgagagagtatagtttatttcagaagtgatggttgcctgttttaagtctgcgggctcaaggcatgttatctttcaagttgttcaatatcaaaatagatgagccaaaaagaagggaacagtgtgcttaaggtttaaaaacgtaatgttgaagttgttggttttagttactttacttttctattttacagggttggtttttgttttccattttgtgggtctaaaatttcttgtaaggctataagatgtacagtgtacccrtataaaattaggattgacttatagaggggaaatttatttggtttattaagatcctttcagatggattttatacctgcttctctatactagccaagtctgtctgagtgaagtggtaatgttatctttaatttacattttaaaacttttacatagttaaattttaataaaaaatctgttcttgccttgggaactaccatagatacgttttcttcagggtacattgagcgaattccccaaattgtattcgtattgttttatttcatttccctgttcccttacccaggttgtcttatgagttc

SEQ ID NO: 183 - DNA, 599 bp, Homo sapiens
tgtgtacatcacatcaatgtgagggggtgctggaagttgaaacagctctgcaaagatcacctacaaaagaatcatgagtcacaattcaatccaactgctatctttgcacttctattagaaagaccttaacattctaaatctgagctctaactccatggcattcatatgaaaatggaattactttttaatatttaaaattatgctctttcaaacacaaaaatctaaaactgaaaactgtaatgaaaatttcttctctctataattcgatcaaataaaaacacacgacactaatgcttattygatctacactcctcccagagaactatgcaattaaattccaactttgaaagcaccacagctgagccaggatgtacagctttttgaggggctcaaaagatccatgacagtttcattcctataaataacaagaactcttaggctgggcgcagtggctcacgcctgtaatcccagcactttgggaggctgaggcaggcagattacttgaggtcaggagttcaagaccagcctggccaacacggtgaaactctgtctctacaaaaaatacaaaaattagccaggtgtggtggtgggcacctata

SEQ ID NO: 184 - DNA, 599 bp, Homo sapiens
cggttaaaaaatatttacagctattgagcttgggctagaatctagaaggagaaaaggaagtggagctagaaagggctttccagactggggtaatggcaaagtcaagaacagggagtatggaaaaaataaaaaagtacatggacctggcagaaatagaggtccatgtggggagaagtgaaaaataacaaagtaggaacaatggttatgaaggccttgaatgttaggcggagttattcctatggggcaaaaagaaagcttaatcttcaacaaaagcctgagctttctgatagcaaagactayaccttatcattctcggaaccagtacaggacaaaactcaaaacccagcacttctaagtactgcataaacctttgatctgccaaaaagcacagtggctaacagcatggccactcagttagtgcacctacgcctgaatttgggctccaccactcaaaagcaatctcattctagcttgacctcttggaagtcatttcctcattgtagttttgataatcatatctattcttcagagttgacataatgaaataggaaatgtaccaggaccgacacagttcatttcaaggaaaacaggtacagtca

SEQ ID NO: 185 - DNA, 599 bp, Homo sapiens
aaattattttaagatatcaaaaaaaaccttgacatgaggaaaaaatgacccttaggcctgcagggcacagaactgctggaacacggccaagatatgagggtctaattcagcagtgaagagaaggttctccagggtcaccatgtactgctggattatttggtgatgctgtggacacacaaaagggggtagttaggtcaggaaaaagcatataagcaatcttgtggaacatttccttcagcagcccacccatgctgcagaatgatgaaaagaggaaggggaaaaaaaagtgctgataggaayaatggacccctgaaaatccccaacattaaaaaaataaaaagggaaatgaaaaaaatggatgctcaatagctttttaaaaaatccagttcttaccaacttctatgtaaacagaattcatgattcaaggcagtggcagcagtgcctgcttgctcctagtactattacggactaggctcagctagctctctccccagtcagggcataatacattgtaactttttaaaaagcagccagacggactgacaggaagataaacagtcagaaaacgaaatactcttgtctatgtgttcctctataag

SEQ ID NO: 186 - DNA, 599 bp, Homo sapiens
atatttctgaatcccagccatgtacaaggtacttgtattagtgctacattagttataagaaactaccacagacagagtgacttaaaacaacacacatttattatcacacggatctctagtttattctgacatgggtctcagcaggctaaaatcaaggtgttggcagggctgtgttcctttctggaggctctaggcaagaatccatctccttgccttttccagcttctataggctgcctgcattccttgactcatgaccccttcctccatcttcatagccagcaatggtggcttctcacayggcatcacccacttcttccataatcacatgtccctctgactcttggctttcctcttctactttttttgtttttttgaaatggagtcttgctctgccacccaggctggagtgcagtggcacgatctcagctcagtgtaacctccacctctcgggttcaagcgattctcctgccttagcctcccaagtagctgggattacaggtgcaagccaccatgcccggataatttttgtttttgttttgttttgttttttgagatggaatctcgctctgttgctcaggttttagtgcagtggtgcaa

SEQ ID NO: 187 - DNA, 599 bp, Homo sapiens
ctcaagcttagtcttggagttattgttgcccatcatgggtagagccaagggaggatgaatatggtccagggttcacccattatgtagatgaactctggtcataatggggttatggtcttgtggtttcctcctctcttaccccaaatgggagttttcctttcaattatctttataacaagtgtggttgcagcttcaaaccacaaaccactaatatttttattggcaagtgcatcctctaatacgtatagttctgttgaatgggtgaaatatttatcaccagatgcatttgtttttgtagtktttataatgcttggaaaggggctgagtgtctctggccttaatcccttctttggctctactgccactgtcctggtttagctccccccaaattttacctgtatagcctctgaactggtctcccagactccaggctgacccctctaatccatcttccacattggcttacagctccatctttctaaagtacagcttttgccatgccattcctttgctcaaaaatcatttattgactcccagttgcagctaattgtaagttcccatcacctggttcctgtagcccttcaggaactttccttcca

SEQ ID NO: 188 - DNA, 599 bp, Homo sapiens
gaaagggtctctttctgcatgtgtgggaagcggccttagatgacctcaggacaatgttcaaccttcagtgtctatgattttcatgttaggtaggggtagatcactattgagaagggctcataaaataatctgatggccttggtgtcctacaagctaagtggaaaccaacagtgtgacatggcttctgaaaagagatggtgtgacctgaggtgacaagaacaaaagcagggcctgaactgagggaggtgtttgtcccgctgtttggggctgcttaaaccacatctggagcttgtgttcagwtctagacaccaccagggaatcagagagccatctatgtggccaggacggggagggaagagttaagaaaccctatcacaggaagaaagtgcagattcatgaccaggcactgcatctccaaagggctcctctagggcagagggcatagatatggtctgccaggactttatgctctcacttacctggacattcacagtggtctgcctgggcaggatttcttctcccttcagaccatgagacccaggactgctccacgtcccccatctctcagaagctaaagacggaagcagcaacagggtgag

SEQ ID NO: 189 - DNA, 599 bp, Homo sapiens
aataagagtagctgggactacaggcacacaccaccacacctggctaatttttacactatttttttccagaggcagggtctccctgtgttgcccaggctggtcttgaactcctgggctcaagggatcctaccacctcagcgtcccaaaatgctggaattacaggcatgagtcaccgtgcctggcctgaagtttttttcaaaggaaaagagaaattttaacaggaggaaaaagaaaaattgagtacaatgtatttgtttgtgtaaataaagacatttctatacattttaaaggcaaacaccygacttgcttttattaccagcttggactccacagcatcctgagtttgctagccagtagagtctttggcaccttcttatctctgcagtgtggacctcaggtcagattctcaccctgttgctgcttccgtctttagcttctgagagatgggggacgtggagcagtcctgggtctcatggtctgaagggagaagaaatcctgcccaggcagaccactgtgaatgtccaggtaagtgagagcataaagtcctggcagaccatatctatgccctctgccctagaggagccctttggagatgcagt

SEQ ID NO: 190 - DNA, 599 bp, Homo sapiens
gcggcctcggttgtcctggccacaaatgcaagaaagcctttacaccccaggctgcaggaggttccctaagtatgtaggttattcatacacacgctccttttttattggaaagttagaaattgcactacattgtaccttgggggtgaaggtgacattaacaaaagcagtttggagtagtggaaaggatactccaaaggggaaggcttgcaggatgtgggttcttgcccttgccctagcccttgtgctgtgtgacccagacacagcctgtgtctggaccccctcttgtagcctgagagggtyggaccaggagatctctaagggacactccagctgtctatgattaagattctgggacaaataaatgtcatgaggcagcaggtggggggactgggtggggggacatggagtaaatgacctctggatgccctcactggggctgggacttgatgatttgtacacagtcaagggaagccacatacaggctaatcttggcctcacaaaggagtgtggttagagttgttgggtggtggcgatgttttcctgcaagcgcacacgatgtttatcgtcttcgtacaggttgatgctgaagccacgaagac

SEQ ID NO: 191 - DNA, 599 bp, Homo sapiens
gattatacagtatttatttttgtctatattttttcactcaacagtatatgaatgagattcatgttgttgcatatagctatagttcattcactcattgacatatatagtattcttttgtataaatatatcacaatttacttatccattctactgttgatggacatgggggttgtttccaggtttcagctatttcagctgctatgaacattctttagatgtcttttggtgaacataggtatacacttttgttacctacctgctcaggagtgcaatttctagaccataatgttcagctttagyggaaaatgctgatagtctccaaagtggttgcagcagtgtatgaaagttccagttgctctacatctttacctaaaacatcaattttaatatcaacacagcatttcattatatggatacaccatacttcattttatcagattcctatagatcagggattctttgccatagacctctggcactctgataaagcctgaggactctgtctcagaatgcgacatagtaaaacgtgggcattatcattaacacactaaatcacaccatccagtagcaggtctaataaccaagcaatttcaaaattt

SEQ ID NO: 192 - DNA, 599 bp, Homo sapiens
tgaatttgtatttaattggtatcacctctgtaaggtgcatatccatcccttgctagaagagtataaatatataaaatacatattactcccaaaacgaacaggattttgtttctgttctggccttttacaagttgtttattaaaaaacattagaaattgtaaaaaaaagattagaatcatccaaaagtcattattaaaggtttattttgtgtgttttgaacctgtttgctatgaatatacttaagaataaaataatgccatgtagggaatatgttttgagcttagacagtatattatgggyatttttcaattcatgcatgactttttatggctatgtggtgtttgaatagaattttctctctgcatagttattaatggaaataattcactctcggtttaaaaaataaactctgtaagacttgtattatccaaaattatttagtttcatataataattggtgtgtaaagtaggcagtggttgtatggttaggtatgagatgtggtagaaaaaacactacagctgatactctggggtataagtgaacatgacaatggaatttcctgaaatatagagctttaacacttcagaacttttatccc

SEQ ID NO: 193 - DNA, 599 bp, Homo sapiens
tttctacacataatgtttacatttaggagaggaaaaatgtatttccatttttaatgttaatgtccatcatagtttgccatttcaattcagtaagtatgtacctctggtaagggatggaaataagaaaaatgactacacagtgctgccctcagggggctcgcctgaatctaatgggtgtgaagatagatacttgcacaatttacaataaaagaccaatttgtaagtattgctggagaaagatgactaattcagccccaaggacagaaggctgtatcagctgttaagaatgaaattacaaargttgaaagattttattttctgtaaccatagcttgacatacagtaaccaaatgccatatttctttaagattgtttgtttgggaaattttgcactctacaattcgtaagatgaacaaacatgtcctgaagatccagaaagagctggaagaagctaaagagaaacttgctaggcaacacaaacgggtaagttttgtgtaaacttgtaagtagttaaagaaaatataccagaaactctggagaggattcagagttcattatcttgatgatatttcattttttctgagcccaaattaatgcaga

SEQ ID NO: 194 - DNA, 599 bp, Homo sapiens
agttcactatctctgggtcggcctcacgtttgcacaagggaagtagcagaagggaagaggacacttgtatttctgtaggagtctctgggggaagagggagaggactatagatacttaacactttccccgtactgtgaggaggaatggggcagtcattcccaagttaacggaatacttctcatagtattaatttagtattttagccattttggagggaagcagtgagtgacttaacatgatactactattcattatgaaagtattttccagtgttatgcatatttaccatagtagaaggtraaaaaaagttgagtttagtgccctataggaacctttcgcaagtttgagtaccagtctccatgtagacatacctgtatacatatatggatataaacactcagacctgaaatactgtaaggtaaatggtgtcatctcattttgtagatgttataaagtgagacatagagaagttaagtaacatagctagtaggttgcatagctggtaaatgaccagagctaggttctgacacaggtctgtcagactccagagacttttctctcatgccacactgtttctagaagtgtgctgtccaacacag

SEQ ID NO: 195 - DNA, 599 bp, Homo sapiens
attctgtctgtagtactaccttaacttcttctccctttccagcggtttatcatgatcttgaccgagcacctagtacgatgcgaaactgatgggaccagtgtattaacaccatggtataagaactgtatagagaggctgcagcagatcttcctacaggtatgtggggaacttcgattaggtaataacactattctcagccacaaagatttttcattaaaaaaaatccttgtcaaatttgttaggaggttatatagtacctgttagaccctttagttcatgtccatttgctgctaccattgstcatcagaatatttaggcttaaactgattctgtaccacttagattcctaattgccacccccccaacaaccccccgccccaccttttttttgagatggcatctcactctgttgcccaggctggagttcagtgacacgatctcagctcactgcaacctccacctcctaggtttaagcaattcttctgcctcagcctcccatattgctgggattacaggcacccgtcactactcctgcctgatttttgtatttttagtagggacagagtttcaccatgttggccaggctagtctcaaactcc

SEQ ID NO: 196 - DNA, 599 bp, Homo sapiens
ccaatataattttctttgtaaaatgattttgacacgaagtagcagaaaaagctccaaacattagaatatgaccagtgtcctggcttcacagttaaactatctttgctcagtttccctacttgcaaaataaagtgaagtaaaatcaaaactctgtggttctttccagctcttaacattctgtgattcttatttttagccctctcctaaaataatgctgtcatcttataagaacttcaacagacttgaggtcttattgcacaagcactttttctaagatgaaaacataccagcatagtggtrgagttctctactttctagttttcttgcaaagcttgacttgatgcttattgacgtagctcctaacaggaacttaaaaacactcctttttcttaattatttcagctgactccttttcctactgtggcattctgttatctgaagtcaaacgtacctgaatgtgtcctattgtgttactcaactcacttctgaatatggttctgttttgcataccaccaacccacataaccatttctgaatctcttgtttcacactgctccagtgacaacttgattcacaggccgaactagtgaggtaagaaa

SEQ ID NO: 197 - DNA, 599 bp, Homo sapiens
gaaactgcaaattccagctacttttgatggtactggtctcatcctttctactttatagtggttaccttttattcctttctaaacttgaggcacgctagagtcagacagaaagttcatttgcactgattaattcatagtcatgattccataaaggtttccaggtctacgacatggctgtggaacggaaggtgcaaaatagcaaaaactgacattaaattaaacttttgatcatggaaagcaaggtagaatgaacattctttaagatttcttaggcagttccccaagaactgaactgtattktagcattgtgtacaacccaccatgtttcaagagttaggtaagtgctgagatttttatctttgtgacacttttatttcatggtactatacaattttctttttctctgtgtgccatgacatatgccatgaaagactgaaaaaacttgcttagaatacgaagagtggtgctaaggtcctggggaaacagcaaggcatagcagcagagagaaatcggactttgttttgggcatgagttagcattgtttaggatcctgctcaagtcatgggctcgactactccagctttccctcttcttaattc

SEQ ID NO: 198 - DNA, 599 bp, Homo sapiens
aaaaacttcaggccaggcacggtgactcatgcctgtaattccagcattttgggacgctgaggtggtaggatcccttgagcccaggagttcaagaccagcctgggcaacacagggagaccctgcctctggaaaaaaatagtgtaaaaattagccaggtgtggtggtgtgtgcctgtagtcccagctactcttatttgtttttacaataagaaaaacaacagcacatgtgtgaggcgaagtaacctctgctggtcatcctcagtgctgggggacaacgcagaggggaggggcagcagtgacsggctgcagcaggcccagtgaggccgtctcccactttgttaaagaggagttagaaatctggattttaatgtgaaatcttccagtttctaaatgtttacaccaatgtttaaaaacacacacaggcggccaggtgcggtggctcacgcctgtaatcccagcactttgggaggccaaggcaggcagatcacgaggtcaggagttggagaccatcctggccagcatggtgaaacctcatctctactaaaaatacaaaaattagctgctgtggtggcacgcacctgtaatcccaactacttggga

SEQ ID NO: 199 - DNA, 599 bp, Homo sapiens
acatggcgagacccccaacccatctctacaaaaaatttaaaaatcagtcaggtgcagtggtgtacacctgtagtcccagctactcatgaggttaaggtgggaagattgcttgagcccaggagttccaggctgcagtgagctgtgatcacaccactgcactccagcctgggtgacagagctagaccctgtcttaaaaataaacaaataaataaaaataaaataaaataatccataattccattacccagaggtaaacatagttatcattttatattgatagtgccagtcctttattttcasaaaaatgagattatacagttttatagtctgatttatttatgcattgaattggcaatacatatacacagttaaatcttcagaaggtacaaaacagcataaagcaaacttgtccatcccacagccatgggccacatatggcccaggatagctttgaatgcggcccagcacatattcgtaaactttctttaaacattatgaaaattttttgcaattttttaaagctcatcagctatcattagtgttaatgtattttatgtgtggcccaagacgattcttattcttccagtgtggcccaggga

SEQ ID NO: 200 - DNA, 599 bp, Homo sapiens
gagctagaccctgtcttaaaaataaacaaataaataaaaataaaataaaataatccataattccattacccagaggtaaacatagttatcattttatattgatagtgccagtcctttattttcagaaaaatgagattatacagttttatagtctgatttatttatgcattgaattggcaatacatatacacagttaaatcttcagaaggtacaaaacagcataaagcaaacttgtccatcccacagccatgggccacatatggcccaggatagctttgaatgcggcccagcacatattcrtaaactttctttaaacattatgaaaattttttgcaattttttaaagctcatcagctatcattagtgttaatgtattttatgtgtggcccaagacgattcttattcttccagtgtggcccagggaagccaaaagattggacacccttgacatgaagtaaaaaataaatctctctcccattctcactctccagaagcaaccacttctatcaattgctcatatatcctcctaaagatatgtcatgtactgaaaagcacatatgtatgatgatcttgaagggaataagtgaaagcacactata

SEQ ID NO: 201 - DNA, 599 bp, Homo sapiens
tctgggccctctgtttaacaagagtattagcatttgtttttatattcaataagtgtataaacgaagaaaacaatttaaggtaagactttctgccttccaagagatggtggtggtttcaaatacttggggcccagaaggaagaatgaagatgttccttctaaaacgcagaggacaattcagctgttccgaatgatggccctgagtgtcagctaaccccctcctgcagctaagtccccctgggtccccctgcaagctcttcccaccccgggtgctgggaccacctcttccctcaatcagacrtaaatcaaaggaccaagattaggaaggatgggtgattttgacctttctataacagagaaaatccagataaatggtcttctctgaaccaccaagttttgcttttctttttctctcaagttggtaataacatgaacaatgacacattaccttagagcttgaactgatggtcgagtagataatctaatgtgggcctgatgagaccctttacagatgaggtagttgagactcagggggcttcagtgactgggccaagattcccttttggctggtaagtgacagcttagaatccaagctgtggt

SEQ ID NO: 202 - DNA, 599 bp, Homo sapiens
ctttctataacagagaaaatccagataaatggtcttctctgaaccaccaagttttgcttttctttttctctcaagttggtaataacatgaacaatgacacattaccttagagcttgaactgatggtcgagtagataatctaatgtgggcctgatgagaccctttacagatgaggtagttgagactcagggggcttcagtgactgggccaagattcccttttggctggtaagtgacagcttagaatccaagctgtggtctttggagtccaaatctctggcctttcctcattctgtgaagayctgcaccccttctttcaggcaggtataagacaccgtgtaaaatgtcaaacaaaagttcagctatctttggctgtgtggagatggaagatatttcaatacaacagaattaataggcttattttgaaatccttctaattcctcagtcaactaatctgagccctagctctgggccctagattgctgggttttcagctgtggctgacaggtgcatactttctgagcagaaaacacgcaaggctgggaagtggcatggttttcataatagccacaggtgttccaatctgcctttctgcttcacg

SEQ ID NO: 203 - DNA, 599 bp, Homo sapiens
gacagcttagaatccaagctgtggtctttggagtccaaatctctggcctttcctcattctgtgaagacctgcaccccttctttcaggcaggtataagacaccgtgtaaaatgtcaaacaaaagttcagctatctttggctgtgtggagatggaagatatttcaatacaacagaattaataggcttattttgaaatccttctaattcctcagtcaactaatctgagccctagctctgggccctagattgctgggttttcagctgtggctgacaggtgcatactttctgagcagaaaacacrcaaggctgggaagtggcatggttttcataatagccacaggtgttccaatctgcctttctgcttcacgctttttatactttaccagacagcaaagacatgttgatgaagactggtttgcccttctgaggttgtacatgactggaaggatttgttaggtaagggaacttgcattttcctgctgtgtttcccctgggaaaacaggactctcaaagacagtgccaaaactttgaatctggaagttctggcctaaagctgagtaaggaaagttttaaaaaatgacaacaacaaaattagggtga

SEQ ID NO: 204 - DNA, 599 bp, Homo sapiens
agaaaccctggaggaagattcatgtgaaagtgatgtattaggaagtgctcccagaaaaaatggtaaggcagtggggcaccgtatcacacaaggcactcggaggagaactttggcttaatcccaccagggagctctggtgagggtcacacccgtgagtcgtcctggtcaggggctgggggctagagtacttatgcctcagcacccatcaattactagaaaaagagtgggtgggggtgggtaatctcccaggtacttccagctctcctgtgtgcggacagaacagtcgccagcagccctggrctctatcctctgacaaagagatgcagatgctggccacaggtaggcccccaatggtaaaggaatctaaatggatatgcactgacaatatcagctcaaagaaggactaattccttccaggctggacgttcagaacacgagaaatgagaacaagagacacataggacaggcctctctggagacggcccagagaggttgagtgacctatcagggtcactcaggtggcagagtcaggattccagccctggtgtatctgactccaaatcccatgtgcccttttctgtgccctactgtctcactac

SEQ ID NO: 205 - DNA, 599 bp, Homo sapiens
cacttgtaattcgcagtgcctcccctgcccactcagggaggtgatgctggctggctttagggacccttcaggtggggcagaacccaggaaggaagtggggtagggagactcatcgtttagatgggacagcctcgggccactcacccccaagggcagttggcaacctgggggtcttgcagctatcagagacaggcttagccaggccaccccctggcttctggctacttccgtgtgcttaggcacctgtcctgctaccctgccagtggcagggtgtatcttatccaggagcagctcaactgyatccaagtcctgggctggagccagtttctcccacctgtccctgggatgcaggagaacatcttccagccatctgcccagagccagcccgagcagctggcagcaggaggagccggggatgtgtgctgttatctctctgcgtcctgagtggaattaccaaatgagtgaactctgccaaggcagggagaggaaacagaactcaatgttctttttcagagttcaaaaatagtcctttttgagttttgaggaagatctagacttaaaaaaaaaaaaaaatcaagttccccagtgctaaattcagc

SEQ ID NO: 206 - DNA, 599 bp, Homo sapiens
tcagcctcctgagtagctgggattacaggcatgcgccaccaagcccagctaatgttcgtatttttagtagagacggggtttcgccatgttggccaggctggtctccaactcctggcctcaagtgatctacccgccttggcctcccaaagtgctgggattacaggcacaagccaccgtgcccagctggatacttgtatttttctaaaatggcatagccttcagttcagatgaggtattctgtttcatgcatgctgaacaaaagaatgtgaataaaactcctggaaagccccatttgctatsgagctacaatagtccaacagccaaagaagtgaattctgagcttaatctaaatccaggtagcttaacagagtcctccacagaggtcagcagatctaggctcaagattaagctctgccactcacttgcatgtagctgtgagaaagttaattgacttctctgaatcttaatttctttatatgaataaaaagggaactaaagagcaccagaaatgaaaagaactgctgaggagatactaccaaaatcctaaagcacagagtacatctgagggctgagggtatacatgagcatgtatctctgca

SEQ ID NO: 207 - DNA, 599 bp, Homo sapiens
cttttattattgaattgtaagtgttctttatatattctatatatcacaggctttttaatttttttctttatgctttctctattttctaaattttctacaatgactatgaggtaccttgatgagcagaatgcataaccagtatgtaccaacatatataaaaatgtagaaactgtcctcatcaaatttatgaatgacatagaactggaaaagatgactaatgtgtcttttgacaaaatcaagatccattcttggaggaatggagcaatgagtgaaatataaattcctcacttcagttaaaamattctgtaagggtgtgatatggtttggctgtgtccccatccaaatctcatcttgaactgtagttcccataatccccaggcatcatgggaggaaccagtgggaggtaattgaatcatggggacaggttttacctggctgttcttgtgctagtgcatacgtctcatgagatccgatggttttataaagggcagttcccctacacatgctctcttgcctaccgccatgtaagaggtgccaatgctcctctttcaccttctgtcatgactgtgaggcctcccagccatgtgaaactgtgagtc

SEQ ID NO: 208 - DNA, 599 bp, Homo sapiens
tggcccttccatttcatatggccccacacttcagggaatttacttactagttagaaaaacaacgaaaaagacacatgaggcaattagcagatgataacgggttacataattatcagttgaattgtatgttacagagactaacttgttataggagcttagagaaaggaaaataggatctaaaatagacaagaaggtttcatggaggagcatggaaagcatggaagagactaggccctgaaatatgatgaggctgttgaggagggactcagactccagagctggataggaccttaattgtcrtgtactgtagcattccctaaaatatgttctgtggaacactagttctagaggatgctaacaaggtattgaaaaaaggggtattaaagacaaatttgggaagcactcagtcaaaatgttgttactgcaagacttctcagagcctttaagaggctttggctgtgattcttaaggagggagatgctgtctgcagggtttccaaacataattttatagaacagcttcataacatactttggaaaatatagtttatttccattctctgattttacagatgaggaaaccaaggcccagaagtgatg

SEQ ID NO: 209 - DNA, 599 bp, Homo sapiens
gcagtgtatgaaagttccagttgctctacatctttacctaaaacatcaattttaatatcaacacagcatttcattatatggatacaccatacttcattttatcagattcctatagatcagggattctttgccatagacctctggcactctgataaagcctgaggactctgtctcagaatgcgacatagtaaaacgtgggcattatcattaacacactaaatcacaccatccagtagcaggtctaataaccaagcaatttcaaaatttcaaaatgagcatcaacataaaaatatgtttatyggcctttgtgctgcttctcccacacaaaatcattttctttgtttctacatttattggcaaaagtcaatgcatattcttgacttttgccaacttttaatggcccatatatatttaaatttgtatgtaagcactcttcctgtgttatggatatgaaccatctctgtgaaatgttacaattgttttcatttgtcattagctttgccattttgtggtctttagaaagaattgatgccctgtgttcagtgcgatgacaaagattggctaaataagtgataaagggaatagtgaaggttgtttta

SEQ ID NO: 210 - DNA, 599 bp, Homo sapiens
caggtgattccagtgtgcagccaaattgggaatgatggatacaaagtgtcttccctggcccagagcatgtgctcagtgagaggggtggtgttgccatgcgtctgattattaccgccacacatccagcttctatcctgtttcccccagcagtgattgtcacagctgtgtcatcactggtgtcctggccttggcctcttcctactctccacctcccaacatgcagcacagagcagctggaaaggtctttcctgaaatgccaacctgaccttcctccctcactggcctgccactctctgaacytcgaggctttcccacctgcattcctctgcctgtaatgttctatagcccttctcttaacttgaccaacacttactcctcctttgcatcccaagttcctcttttatgctgtcttaggactgaactttttcctctaccacactcagcataccctgtgctggtttttcattgtgtgggaacttgcttggctcctctctcccccagcacactttcagctccatgagcggagacccagtctttcttattctccactgtccccaccagagcctagcacacagtgctggggtgggcctcattacatg

SEQ ID NO: 211 - DNA, 599 bp, Homo sapiens
aaaaaaaaagactaatttacatcaatcgttgttatgcaacagctattgccatttaatggagaaagtgaccaccatttcccctgcattatttctaccatataatcagttcctaataacctattattgtcctcccataaaaatctctgcatcctaatgtcttggcctgctcatttcattgttggtagagcctttctttcctataacctacaataccctcttgtcagaaaagcatcctgaacacaataagtatgatgactgacataataattttacattatactctacccctgctccaggctytctaattcagaaaatagaatattgttaatatcactccatcaggctagaagagaccttggggaatgtcttagtgcttctctatcatttgagtttgaagatacttttttaacagctataaattccacagtccttgcaacatacaaagtagatagacttttcagtaaacactgagaaattttatgtccaaaaaatgaccaaaaattcagtaacatcttaaaatgaatttgcattttatttgtcacgcagcttgacatacatttgaaaagtagtcatatatgcatatatacacacacacatac

SEQ ID NO: 212 - DNA, 599 bp, Homo sapiens
cctgaagtgtggggccatatgaaatggaagggccatatctgtatagcacagtgtagttcacaactgcttatactatcaccgtcctatataactcattctgcagtactgggaggtaggcaaggcaggcattattattccaattttagaggtgaggaaatgaaggtacagaaaattttagtgactagctcaagtgtgacttctacttcaacttgctttcttgctcctttccaataagtaaaagggaatgtggcactgagtagtaatgacaaaaattgatcaaatttggactacagttgaccygtgagcaacgcgggtttgaactgtgcaggtccacttacttgtagatctttttgtgcctctagcactcatgcagcaacaagaccaatccctctttctcctcagcctactcaatgtgaaggtgacaaagatgaagacctttatgacgaaccatttaacataaagaatagtaaatatgtattacgattttctttttttcttttttttttgtagggcagggtcttactatgttgcccagactggtcttgaactcctggcctaatgtgatcttcctgccttggccctccacaatgctgggatta

SEQ ID NO: 213 - DNA, 599 bp, Homo sapiens
agggctcggttgcacagcatcattgaatcaagtggattaaatgtgttttctgcatctctgagtcccttggaacctcctaaccacatacgcctgggtacagagaaaggcccagagacaccctaggtcccctcctagcgccccttctggaggaaggctaccacagggaagaagtttccttgggcactttgtatgtcactttccttctgggaagcagcctggcacggtgaacagcaagcatggggtttactggacttgtggccctgagcaagccactgatgtcccagcctaggcctgcccacrtgttcagtagaagtgctgacacagaggagcggaagaaacagggctgggtttgaatccaggctcaatcaacttgagcggattatttaatatgtatgagcctcaatttcatcctctataaagttgggataaaactgcctactctccaggcctgttctttgcattcaaaattagaaataaatttgcacgttgaatgtcctgctcagcagatgcccaggaagaggggcagtgattgtcattgttactgatagccagtagagggcaccaaaccattctttaatgaaagcggcctcggttgtcct

SEQ ID NO: 214 - DNA, 599 bp, Homo sapiens
tctatagggggtccttcccattttgacctttttatgtctttctacaaattccattttgcacttgtccctccctagctcgaaacattctctctctctttaggatacaggtcacatgcctcaccccagcattcaaggctctgctcagctagccccagtccacttccttagggtacttcccttcttctccttcacagattatttgttctggccagacagctgctctctgcattgcttcctgaacattctatatacagttgcacctccatgcctttgcttgtgcccttccctttgccatttatrccctccctatttatttggatgaacaaccatgagataatttgtggagttaatgaaaatttatgagcaacaattatgcacccaactacattaggaggtactagcagaaaagcacaaaggactctaattcaaagtagtcagggcatggttctaaggaaaaaataggcttggaatatgtcctaaaaaaagagaatgctgtgggaatttgagggaggaagtaaaagtgtctatccatgaatatagtaaatatttccatctattgaggtcatctttattagtcttttaacaatgttctatcattt

SEQ ID NO: 215 - DNA, 599 bp, Homo sapiens
cctggaactaactgtgctgaccacacagttgtccaacaaaagtcatcacatgatggtactgcaaaaatattattttatacctgtttcctaaaaaccagtcatactgacaaacactccacatacctgccttcacaaataccaaacttggctaaatctcaaagtaaaagttcatctctcattcagaatgaagaaactgaagataccttgtgaactgagctgcccaaggtccatctcggcatgaggagtaaaccgcacttctaaagtggccacaggagcctctgtcacccaggcaggaaccaygctgcggggagctccatcagcccttccagcagggcagtgaggggccatgggctgtgtctctgccctgcttccttgcaagactgctgctccttccactctttctagcttcttgtctgtacctttctctgaaaagctcttctctgggcaatatgggccaatttgttcttctgccacgctggaactctgatcacgtcttgattccaaaccaaaatccactgctatctccctgtgcataggaaatgcttgctgaattctggctgtgtcactgtgtgtcaggtagttttcttctgaagttctgt

SEQ ID NO: 216 - DNA, 599 bp, Homo sapiens
acttcaatttcctcatcccaaaaatgggggataaattatattacttaagacagttaaggcgattgtatgaaatgagtgtaaaaatgttttaccgtactacctggcaaacagcagacacttagtaaatattgtttgttgggcacttgataaacgtctctcaagttcctactgtgtgccagttgttgtgttagctgcaaaggatgtggagatgagattctgtctcttcctttaaggaaaggggatggtgttattttcttcctaccagatgacaagttaatactatttaaaacatgatttctrttaatttgtgggagattgcatttttttccccatcagatcattgtggtaaaataacttgtgctcttttcttattccgtggtttgttttgttttgttttctataggattttgtttgtttttcacaaaaatggtcatttgagctgtaaggcaaaagtgcagcctcctaggctgaatggtgcaaagactggagttttttccacaaggagccctcatcgtcccaatgcaataggactgaccctggccaagctggaaaaggtagaaggtaacccatttcatttctacttttacctttttcctcac

SEQ ID NO: 217 - DNA, 599 bp, Homo sapiens
gttttctcatgtttcttgtcttgaagaccactttccttttccttctctgtgagaaaaatccctttgccagtccttcaccaggtgagtcctcacaggaatgagactaactcaaagtcagggaaatccatcatacaaaatacacctagcaggaatttctcaaagaccaagtctagaatagtcacgcagggcctcagcagaaacacatgaggtgagcttgggcactcaaaagacagtcctcttctatgtgtcttcaccaaaatgaaatgaaaatgccactggttctccccaccacctcccaartggtaatatttggaacacacacacactttcaaagctaaacaataattctcaaagccagtgtagactctagactactagaagacatttttacaaatgcacataatctgcacagattctcccgcccacatggatatccatcttatacgtgcccctttttgctcacaccagttggaattgtgtttctatcacttaaaatggtaagagtcccaaccagtgcacctcatctccctgtattggttaagaggcattcagccagccgggcacggtggctcacgcctgtaatcccagcactttgggag

SEQ ID NO: 218 - DNA, 599 bp, Homo sapiens
tgaaaccacagctgcttcaagaacccagacaggcatagcattatacatggactctggagtcacacagacctgggtttgaatcttgcccctcttgcttcttgtgtggccttgggcaggttgcatagcctgcctggcactccgtttcctcatctgtgatacagggatgggttgtaatgccacttacagagtctttgtgaacattaaattagataatagacacaaagtacccagtatggttttggcacatgatacctgctcaggaaattaactcttagaatagcatgtgaatcccctagttcrccttcccagtgattctaaaagttaacaacttcagagtttacatctgttatctccttaggagcagagattcttataatcctcattttacaagccaggaaacggaggctcagggaagtaaatgtttttgaaccagggtattcagctaaggtaagggcaatggttagcatgggctccatcttggactctggggggatagtgtgtggagggaaggggcaactccctggcagctgccaccacagtgacctgctcccagtcctcttttgctgacattctgccctgggagttaagtttcagctgac

SEQ ID NO: 219 - DNA, 599 bp, Homo sapiens
gagggggtcaaggcaggtggatcacaaggtcaggagtttgagaccagcctggccaatgtggtgaaacttcatctctactagaaatacaaaaattagctgggcgtggtggcacatgcctgtagtgccagctactcaggaggctgaggcagaagaatcgcttgaacccagaaggtggaggttgcagtgagctgagatcatgccactgcactccagcctgggtgatggtgtgagactccttctcaaaaaaaaaaaaaaaaaaagtatggaaatgtcacccacaccccctgactgagagacaasactcccatggatgatgggctagtcacagaatgggctggttagctttggattgctttgcaattaaatgcatggtaaaatcattgcactgtcttgttcatagcatttctccttttgggatctgagatctggtctaaaaatgaaacccttaatattgggggaatccattttaccttccaggtgtgcctgcttattaggtcctagaaactgctttcctcaccctgttctacaaagggctccactctaaagccagtaatccaattaagaaatataaaaactggcaaatgaaaaatcctacagct

SEQ ID NO: 220 - DNA, 599 bp, Homo sapiens
ggcagccaggcccccaccacccattctcatcattccacacaaacgtgagcccctaccatgaacaagatgttgcatggagcactggcagaagcagagtgagttgaaggtggcttcctcccaggactgcatggtcacagaccccagcaccagcaaagtatggtcatcaggaccacaggaagggccagagatgggctatggggcactggggtcaggaagagtgccccatcttcctgaagggtgatactggacctggtccttggagaataagaaagatctgatgaagcaaaaccctggaaggarggtatccaagatgaagagaaagtatgaggtgcagtctggaggcaggaaagatgggctgcatcggaatcacaccaggtagaatggttggttggttgaattgggaggtggagctggagaggtatgtagggcagatggcaaaaggccttaaattcaggccaaggagagtggacttgattatggaggcaacagagactgagggacaagagctgggattggaaagaggccccaagcatatatgaagctcccctactcatgcctgccccacaccttttcatgggatgttgtacagcacactccca

SEQ ID NO: 221 - DNA, 599 bp, Homo sapiens
aattaattgtttcttttcagtctcttccattccctccacccctaagaatgcaagcattgtgagccccaggtccacatctgtggctggctcccctgtatcgcagttgtatttgcacaatgcccagcactaagtagctgcttcataaaatgagtacgcacatgggggtagacattgtgcagaggacttcaaaccattccaaatcctggacttggagctcagtccaggtttcactgaaaactggaagtccagggaggcctaaggggcatctagattatagaacttctgaattttgaaaggatygcgtggtcagagggatcctggagggggtggcaggagagaaggtgaggacgagggcagggagtggggatgggcagcagacctgggtggacacctagcgaggtgagtcattgctggtgtccacttcctcatgtacccacacccctcccaggggaggacctggaggatggtgtgagggatggggaagggagtctgaacggtacagaaatcagtcctgccactcctccagctctggactgggcagagctggggaaagctctctagcctcccagagctggactgtcttcatgaaaaaccaggct

SEQ ID NO: 222 - DNA, 599 bp, Homo sapiens
agtctctactaaaaatacaaaaattagccaggtgtggtgatgcgtgcctgtaatcccagctactcgggaggctgaggcaggagaattgcttcagcccaggaggcggacactgcagtgaaccaagatggcaccactgaatcccagcctgtgcaacagagtgagactctgtctcaaaaaaaaaaaaaaagtgttgggactacaggcatgagcacctgcacctggccagtccttttaaatactgtactaaagggaagatgctgatttgggttgcaatacgctttcacactctgccatctgtgyggggatgcactgtggagtgatgcaaagaactggcctccacatttggagagcacgtctgaggggccaggagacctctccatgcctctcatttcattctagaccatttaatctgctgcaagtgaaaactgattgactcctttacattctaactaggttttattattttttgtgaatttttttgtgtctctaggtatgagagaacagaaatagagatttgggtggctttagctgtttaaaataaggtgaccaaacctcctttctgcctgttcctgctatttcaaacaccactgttctccaca

SEQ ID NO: 223 - DNA, 599 bp, Homo sapiens
tggtaaataaaatcagaagaactttacatcataaaagtctggtaacatgtcagtcttttccccaaatactaaaaaagaagagtgcaatgtttctccgtatttccgaagtttcaactctgcgcccctctgctttttcctcagcccatattgactcctgtcttccctgaaagtagctgcattgccacactttatattttattatatttcatcctctagtgtttcatgtctgtgccctaactaaaattgtaagcaccctaagattgtgttataaatctctgaatttttccctatactgccacsctagaacattaaagaccgcttgatacccaggaagttcttaatggccatttgattactcaaatgttaacatttgtttcagctaaatttattaaactgtttgattgtctcatactgtgaaaatacctggatttaataaaaattattttcaatttatatttctttagactactatgctgccagaaaaacacatagttctctgggagagtagataaaggaagagagggactaaagtctagtaacctaatataaaggttgcaaaagacttgatgtatttttctatatatgaggctgagtgggta

SEQ ID NO: 224 - DNA, 599 bp, Homo sapiens
catggagtagtaccagtctgtggcctgtgggttgaagacccctgaagtaggaagacttaagaacagcagagtggcaagcctgtaagagcaagcggtttacagtttaaacgcacagcatcctaacttagcttgtagacttcaggaaatgcttcccgggccaggttcagtgtctcatccctgtaatcccaccacttggggaggctgaggtgggaggatcccttgaggccaggtgcttatgaaataggccttagctataaatttaatggaatgcctacattcaaaaaatacacgcgtgcgcaygcacacaaacacacactattttcctcaatagtaagaacatctagtagcttttttttttgagacaccctgtctgtcttctgagacggagtgcagtggggaacaatcatggctcactgtagcctcaaactcctaggttcaagtgatcctcccacctcggcctccagtcttctgagtagctaagactccaggtgtgcgccaccacacctggctaattttttttttttctattttctgtagaaacaaggtattgccatgttgcccaggctagtctcaaactcctgggctcaagcaatgccccc

SEQ ID NO: 225 - DNA, 599 bp, Homo sapiens
actgctgtagcagcagggagcctgcaccagcacagacacactgatgggacgatagaaacgagaatgtgaatgtgtaaaccaccagagccaagagtattttattttcttctcagtggtatcatattctaaataatggatgtttttctctaatttttaactgagaatgatcaaaatgcatgcattaggcccaccttcatcctcatcctcatcttcatcttcttcatcaagtccaaattcttcttcctaaagaatagcaaacagcataacattaaattaattctgccaaattgaaaagtgcawatagtacaaattctgccaaactaagaagtacaaataaatgcaacaatatcaatcaagcaggaaactgtaatgttatttttatctactttgatgtaccaagacccactgattttccttaacgagctttataatttttctaagaaaaattttttttagaagcaagcagttcatttgctagatgtttaattagtatataactctggataattcaagaaaacatcttaaaatctcaaatttcaaaacagatttagacaaaaatttctatatcccattcatatacaaagcttctttcagaactt

SEQ ID NO: 226 - DNA, 599 bp, Homo sapiens
aatatctgtaggaaaaagaaaggcactgtagcataagtaagacttcaaaggatttagagtcagatgaccacctggctggatgctattagacagattatctaatcttaaagagccctagtttcctcacctatgagatgaggattgctactttgtcaaacccacaaagccactgtgagataaggtacgtgaacactgtttactaactgtaaagcactgcacaaacactagttgccataaagcttatatagtaatgtctacagtgctactgtgtttgccttttaacctatctgacttgacctycccaattgaaacattaagttctttagggcatggatttctactttcatctgatttgtttatcactatagctctagcacctaggacaatgtctggcacatattcagtgctagaaaatatgtccctgaataatcataaaatcttctagtacttgctatccaagtttgttagccattacaaattgataaaattcaaggtggatcagctactgaatttttctgagaaaactacctaaggcccagttgagtacaatccagtgggagagttcaagttctgggaaagaacaagcaaaataactgctc

SEQ ID NO: 227 - DNA, 599 bp, Homo sapiens
atccactcaaggtggggagggatggcatcaatgagtagtctctgggcaagacaaagagcagagagttaccccttaacagtcaggcctgttaagcttttttttcagagctcactgaatgtgaccatacttacagagggttttctgagttaggcctgctggacttgcatcagaaattccctcagagatcccctccacatatacaaacacacacacaaagacaagacggacagaaggccttccaaaccaagatccctaaccaagaattccaagagtattccttccaaactatcctattctccrtctgagaagtctccccgaaatcttactgattgaggagaagtctccagaaccaagactcttcctactaattagggagagtaaaccgagacccctgaagaagctgaaccaattgggagaaggaaaggggtgttggcagcacctagactactcaccaaatcagacaccccataatggggctgcagctgcagacaatccatgatggggctacaaacagacactgtgataaggttacagttacaggcaccccatggtggagctacagagggacaccttgccatggagctctagttatgagatat

SEQ ID NO: 228 - DNA, 599 bp, Homo sapiens
ttttgaaattttgcttgtatgaaatgtctctattctattctttcatttagctgggaattgaattctaggtcaaaaataattttccttcagaattttggaggcaatgttccatcatcttctcacttccagtgttactttctgagaagtcttcaatcactataattgctgaccatttttgtacgtgacctggttttgctgtttggaacctcatagaatcttttctttatcaccagtattcttgtactttgcagtaattgttttgtggagctttttgtttgtttgtttgttttttaatctatrgacctgattctcaaggattctttcattctggagacttatgcccttgggttctggagaaatcaatctctttttaaagaaaataatttttcaccttctttttttcactgtctggaagtactattagtcaaatttactgaattcgtccttatttttcttacattttctccactattttcatctctttgccatttgttttctattttcggacagactgccttgttgataagaggataagtgcagtctttcagatacattttcttttattattttggaaatcaaaacatttaacactactcaac

SEQ ID NO: 229 - DNA, 599 bp, Homo sapiens
caatatgtttcttattaagtaaccacatccattactactgtgcaggcaccatgccatagcacttacctgcacagacaggccttagaggctctttattatgttgtaaccagggttggagtgattatgctgataccttatgaacattagccccatttataggggatcttgccccattgcacagatgaggtcgctgaggctcaggaagatgacttcccagaggaccctgtgcagtgtgtccaggacccgaatgggagcccagacctgggtgtggcctcgggccttgctctcacttcactgacwtgaggcccctgaggtttggcactgatttgcatcagctcctacttgttaccaggattacgttctgtgccaggcacagagatgtatcagtaggtgagtgatgtgagaacaaggaagggacaagctagctaatgatttaattaagtacataaattgcattgagattgggagatgtctggccttgccacagcgtgcaggaagtccttctcagaggggccgggctgccccaggcagtttcagatgtcccaaagggagtgacactgcaggcagggtccctccctcttgcctgttgtgacccaggg